SEQUENCE LISTING (91 sequences)

SEQ ID NO: 1 - DNA, 1698 bp, Paraburkholderia tropica
atgcagaagtcaggtgcaggcgaggcccgtcgtgagctcgacgcggtctatgaagtcagcaagacgctcgtgtcgtcgctggatgtcgcgcaaacgttccggacgtcgctggagtatctcctcaatgcgctcgactggcggcgtgcgttcgtcgtcgtggcggaactggacggcgagttgcacgggatgtgcagcgcggggctgtcgcgcgacgagcatcggcgtatccgctttcatccgggagagggcatcgtcgggcgggtgttcaagagcagcatgcccgcggtcgtgccggacgtgcgtgacgagccggtgtttctcgatcgcaccggcagcgcgggcgagcccggcgaattcggcgacgcgcgcgtcgcgctgctcgcgacgccgatccgcgtggagcgcaatacgctcggcgtgctcgcggtgttctgcatcaatccgggcggcacgcgcatgttcgcaggcgacctcgatctgatgaagatcgtctcgaccttgatggcgcaggcgctgctgcttcatcgcagcgtgagcgccgcgcacggccgcatgcaggaagaagtgcggcgcatgcagaaggcgattcgcccggcgccgccgctcgatcacgtggtgggcgtgtccgagcccatgcagcgcatcttcgagcaggttcatcaggtggcgcccgcgcgtaccacggtgctgctgcgcggagaaagcggtaccggcaaggaagtgatcgcgcgcgcgattcatcgcctttcgccgcgcaacgagcaggcgttcgtgtcggtcaactgcgcggccttgacggagtcgctgctcgaaagcgaactgttcggccacgaaaagggcgcattcacaggcgctcaaggccagcgcaagggccgcttcgaactcgcgcacggcggcacgctctttctcgacgagatcggcgacatctcgccttcgttccaggcgaagctgttgcgcgtgctacaggagcgcgagttcgagcgggtcggcggcgcgtcggcggtgaaagtcgacgtgaggctgattctcgccacgaatcgcaatctcgaacgcatggtgaaagaaggcgaattccgcgccgacctctattaccgcatcaatgtggtgagcatccagttgccgccgcttcgcgagcggcgcgaggacattccggcgatggcggagtattttctcgatcgcttcaaccgcgacaatagccgtgcgctgcatttcagcacggacgcgatgcgcgtgctgacgagctgctactggcccggcaacgtgcgtgagctggagaactgcgtcgagcgcacggcaacgatgacgcatggcgacctcatcgatcgtctcgcttttctgtgtcaggaagatcagtgcttgacgcgggtgctgcatcacatcgagcgggatgacgccgtgcggccggcgcgtctcgccgatattccgatccgcgaaattccgggcatgccgggcggtgcgccgcgcgatgctgacttgccgttcgcgttgccggacgatctggccgacgacagcgtcgccgatggcttccattccggcgatgcggaagaaggcaagccggagggcgagcgcgagcggctcatctgggcgatggagcgctgcggctgggttcaggccaaggcggcgcgtctgctcaggattacgccgcggcagatcggctacgcgctccataaatacggcatcgaggttcggcgcttctag

SEQ ID NO: 2 - DNA, 1884 bp, Azospirillum sp.
atgccgagtgctgcccagtccgcgtcatcctccagcctggagctgctgacgatctacgaagtcagcaagatcctcggatcgtcgctggatctggaacagaccctgcgcgaggtgctgcgggcgctgtcctaccagttgcagatgcatcgcggccgggtctacctccagggcgaggacggggctctgcggctcgtcgccgcgcaggggctgccgaaggactcgccggcacaggagatcgactacaaccagggcgaaggcatcagcggccgcatcctgaagaccggcatgccggccgtcgtgcccaatctggccgaggagccgttgttcaacaaccgctccggcgggcgcgacgatctcgacgaacaggtggcctcgctggtcggcgtgccgatcaaggcggccggcaccgtcatcggcgtgctgaccatcgaccgcatctccgacgacggccccagcggccatttcggctccgacgtccgtttcctgaccatggtcgccaacctgatcggacagaccgtgcgcctgcaccgcacggtggcggaggaacggcgcttcatgatgcgcgagaccttccgcgtgcagaaggagctgcgaccggtggtggcccccgtcaacgacgtggtctgcaccagccccaacatggtcgacgtgctggcccaggtgcaccgggtggcgcccttcaagtcgaccgtgctgatccgcggcgagagcggcaccggcaaggagttgatcgcgcgcgccatccacaacctgtcgccacgcaaggatgctcccttcatccgcgtcaactgcgccgccctgcccgaagcgctgctggagtcggagctgttcggccacgagaagggatccttcaccggggcgcagaaggaccacaagggccgcttcgagctggcgtcgggcggcacgctgttcctggacgagatcggggacatctcctccaacttccaggccaagctgctgcgcgtcttgcaggagcaggagttcgaacgggtcggcgggtcgaagacgatcaaaaccgacgtgcggctgatctgcgccaccaacctgaacctggaggaggcggtcggccacggcaagttccgcgccgatctgtatttccgcatcaacgtggtgacgatccacctgccaccgctgcgcgagcggcgcggtgacatcgccacgctggcgaggcatttcgtcgccaagttcggccgcgacaacggcctcaatctcgatatcgccgcggaagcgctggaggtgttgaaccgttgcacctggcccggcaacgtgcgcgaactggagaactgcatcgagcgcgccgcgacccagtgccgcaacggcaccatccgtgtacccgatctctcctgcagcatgaatctctgcaattcgtcggtgctgttccagtaccgcaccatgggcgcggccgtcggcggactggccccatcgatgagcggagccgtgaccaacccggcgcagggccgcacgcagccggccgtcaacgccgctatgccgtcgccggtgccggcaccggcaaacggagcggcggcaaacggagcaccgaacggggcacaatggccggcctgcgcctcgggctgttccgccggcccggcaccggcctgtggcgcctccaaaccgctgccaccctcggccatcgtcccgctgcccgcccccctgccggcgtcctccgccccggcaatcccctccgcccctgccccacaaccggtgtccgccgggccggatttgccgttggatgagccggagtcgggtccgttgcgcgaccgcctggtctgggcgatggagcggaccggctgggtccaggccaaggccgcccgcctgctgggaatgacgacgcgtcaggtgagctacgcactgcgcaagtacaacatcgagatcaagaggttctag

SEQ ID NO: 3 - DNA, 117 bp, Artificial
  Synthetic lil promoter
cataggtgctaggatccgcggccgcttctagagttgacggctagctcagtcctaggtatgctagctactagagatccttatggatttaatttaaactttttattttaggaggcaaaa

SEQ ID NO: 4 - DNA, 400 bp, Artificial
  cspE promoter
gcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttg

SEQ ID NO: 5 - DNA, 400 bp, Artificial; identical to SEQ ID NO: 4
  cpsE promoter

SEQ ID NO: 6 - DNA, 500 bp, Artificial
  infC promoter
agcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgc

SEQ ID NO: 7 - DNA, 313 bp, Artificial
  rm5 promoter
ggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaac

SEQ ID NO: 8 - DNA, 2751 bp, Artificial
  Mutated glnD gene including promoter and terminator sequences
tgaaaagccggcgcccgccggcttttttattagatagttttttcttatggtgacgcgatgagcaactcattacctgacacagcctcccctcttctgcccgtcccgccggaacatccggtgagctggccgcagggcgatctgaactgtgctgcaattaaggcgcacatcgataccttccagcactggctgggcgaggcgtttgactccggcatcgccgcggagcagctcattgcggcgcgcaccgaatttatcgaccagctgctgcagcggttgtggatcgcctacggttttgaatccgtctgcgatctggcgctggtggccgtccttgattatggccgcggcgagctgcacccgctctctgacgtcgcactgctgatcctcagccgcaaaaaactgcctgacgaccaggcgcaaaaggtcggcgaactgctgacgctactgtgggacgtcaagctggaggtgggccacagcgtgcgcaccctcgaagagtgtctgctcgaaggactttcggatctcaccgtcgccactaacttgattgaatcgcgcctgctgatcggcgacgtcgcgctgttccttgaactgcaaaaacatatttttagcgacggcttctggccatcggaaaagttcttcgccgccaaggtggaagagcagaacgtccgtcatcaacgctatcacggcaccagctataacctggagccggacgtgaaaagcagccccggcggcctgcgggatatccatacgctacagtgggtggctcgccgtcattttggcgccacctcgatggatgagatggtcggcttcggctttctgaccgaagccgagcgcaatgagctcaacgagtgtctgcatcagctgtggcgcatccgtttcgcgctgcatctcgagctcactcgctatgacaaccgtctgcttttcgaccgccagctcagcgtcgcccgccggctcggctatgaaggcgacggcaaccagccgattgagcatatgatgaaggacttcttccgcgtcacccgccgggtgagcgagctgaaccagatgctgcttcagctgtttgaagaggctattctcgccctgaccgaggatgaaaaaccgcgcccgatagacgatgacttccagctgcgcggcacccttatcgatctgcgtgacgacacgctgtttattcgcgaaccgcaggccattctgcgcatgttttatatgatggtgcgcaacagcactatcaccggcatctactccacgacgttgcgccatctgcgccatgcccggcgccatctgacccagccgctgtgctatatcccggaggcgcgcacgctctttctcagcatgctgcgccatcagggggcggtcagccgcggactgctgccgatgcatcgccatagcgtgctgtgggcctatatgccgcagtggtcacatatcgtcggccagatgcagttcgatctgtttcacgcctacaccgtcgatgaacacaccatccgcgtgatgctgaagctggagagctttgccaaagaagaaacccgcagccgccacccgctgtgcgtggagctatggccgcgcttaacgcacccggagctgattttaatcgccgccctgttccacgacattgcgaaagggcgtggcggcgaccactcgatcctcggcgcgcaggatgtgctgaagtttgccgagctgcacggactgaactctcgcgaaacgcagttggtcgcctggctggtgcgtcaccatctgctgatgtcggtcaccgcccagcggcgcgacattcaggatccggaggtgattaagcagttcgccgaggaagtgcaaacggaaaatcgcctgcgctatctggtgtgcctgaccgtcgccgacatctgcgccaccaacgaaacgctgtggaacagctggaagcagagtctgctgcgcgaactctatttcgccaccgagaaacagctgcgtcggggcatgcaaagcaccccggatatgcgcgaacgggtgcgtcatcatcagctgcaggcgctggccctgctgcggatggacaatattaatgaagaggcgctgcatcagatctggaaccgctgccgcgccaactatttcgtgcggcatacgccgacgcagctcgcctggcacgcccgcaacctgctgcgtcacgatctgaataagccgatgattctgctgagttcgcaggccacccgcggcggtacggagatttttatctggagcccggatcgcccttatctgtttgccgcggtgtgcggcgaactggaccgccgcaacctcagcgtccacgacgcgcagatcttcaccacccgcgacggcatggcgatggatacctttattgtcctcgaacccgacggcagcccgctttccgctgaccgccacgacgcgattcgccacggtcttgaacagacgataactcagcgcagctgggaacccccggccccgcgtcgtcaggcggcaaaactgcgtcacttctctgtgccgacagaggtgaatttcctgccgacccataccgatcgaaaatcgtttctcgagctgattgcgctcgatcagccagggctgctcgcccgcgtcggccaggtgttcgccgacctcggtatttcgcttcacggggcgcgaattacgacaattggtgagcgagtagaagatttatttataatcgccaccgccgaccggcgtggccttaataatgagctacaacaagaagtgcaacaacggttgacagaggccctcaatccaaacgataaagggtgacgtattttttttagtgaatggaaagaaaca

SEQ ID NO: 9 - DNA, 1776 bp, Artificial
  Truncated glnD gene including promoter and terminator sequences
tgaaaagccggcgcccgccggcttttttattagatagttttttcttatggtgacgcgatgctgcttcagctgtttgaagaggctattctcgccctgaccgaggatgaaaaaccgcgcccgatagacgatgacttccagctgcgcggcacccttatcgatctgcgtgacgacacgctgtttattcgcgaaccgcaggccattctgcgcatgttttatatgatggtgcgcaacagcactatcaccggcatctactccacgacgttgcgccatctgcgccatgcccggcgccatctgacccagccgctgtgctatatcccggaggcgcgcacgctctttctcagcatgctgcgccatcagggggcggtcagccgcggactgctgccgatgcatcgccatagcgtgctgtgggcctatatgccgcagtggtcacatatcgtcggccagatgcagttcgatctgtttcacgcctacaccgtcgatgaacacaccatccgcgtgatgctgaagctggagagctttgccaaagaagaaacccgcagccgccacccgctgtgcgtggagctatggccgcgcttaacgcacccggagctgattttaatcgccgccctgttccacgacattgcgaaagggcgtggcggcgaccactcgatcctcggcgcgcaggatgtgctgaagtttgccgagctgcacggactgaactctcgcgaaacgcagttggtcgcctggctggtgcgtcaccatctgctgatgtcggtcaccgcccagcggcgcgacattcaggatccggaggtgattaagcagttcgccgaggaagtgcaaacggaaaatcgcctgcgctatctggtgtgcctgaccgtcgccgacatctgcgccaccaacgaaacgctgtggaacagctggaagcagagtctgctgcgcgaactctatttcgccaccgagaaacagctgcgtcggggcatgcaaagcaccccggatatgcgcgaacgggtgcgtcatcatcagctgcaggcgctggccctgctgcggatggacaatattaatgaagaggcgctgcatcagatctggaaccgctgccgcgccaactatttcgtgcggcatacgccgacgcagctcgcctggcacgcccgcaacctgctgcgtcacgatctgaataagccgatgattctgctgagttcgcaggccacccgcggcggtacggagatttttatctggagcccggatcgcccttatctgtttgccgcggtgtgcggcgaactggaccgccgcaacctcagcgtccacgacgcgcagatcttcaccacccgcgacggcatggcgatggatacctttattgtcctcgaacccgacggcagcccgctttccgctgaccgccacgacgcgattcgccacggtcttgaacagacgataactcagcgcagctgggaacccccggccccgcgtcgtcaggcggcaaaactgcgtcacttctctgtgccgacagaggtgaatttcctgccgacccataccgatcgaaaatcgtttctcgagctgattgcgctcgatcagccagggctgctcgcccgcgtcggccaggtgttcgccgacctcggtatttcgcttcacggggcgcgaattacgacaattggtgagcgagtagaagatttatttataatcgccaccgccgaccggcgtggccttaataatgagctacaacaagaagtgcaacaacggttgacagaggccctcaatccaaacgataaagggtgacgtattttttttagtgaatggaaagaaaca

SEQ ID NO: 10 - DNA, 2208 bp, Artificial
  glnD-ACT1,2 truncation  including promoter and terminator sequences
tgaaaagccggcgcccgccggcttttttattagatagttttttcttatggtgacgcgatgagcaactcattacctgacacagcctcccctcttctgcccgtcccgccggaacatccggtgagctggccgcagggcgatctgaactgtgctgcaattaaggcgcacatcgataccttccagcactggctgggcgaggcgtttgactccggcatcgccgcggagcagctcattgcggcgcgcaccgaatttatcgaccagctgctgcagcggttgtggatcgcctacggttttgaatccgtctgcgatctggcgctggtggccgtcggcggctatggccgcggcgagctgcacccgctctctgacgtcgacctgctgatcctcagccgcaaaaaactgcctgacgaccaggcgcaaaaggtcggcgaactgctgacgctactgtgggacgtcaagctggaggtgggccacagcgtgcgcaccctcgaagagtgtctgctcgaaggactttcggatctcaccgtcgccactaacttgattgaatcgcgcctgctgatcggcgacgtcgcgctgttccttgaactgcaaaaacatatttttagcgacggcttctggccatcggaaaagttcttcgccgccaaggtggaagagcagaacgtccgtcatcaacgctatcacggcaccagctataacctggagccggacgtgaaaagcagccccggcggcctgcgggatatccatacgctacagtgggtggctcgccgtcattttggcgccacctcgatggatgagatggtcggcttcggctttctgaccgaagccgagcgcaatgagctcaacgagtgtctgcatcagctgtggcgcatccgtttcgcgctgcatctcgagctcactcgctatgacaaccgtctgcttttcgaccgccagctcagcgtcgcccgccggctcggctatgaaggcgacggcaaccagccgattgagcatatgatgaaggacttcttccgcgtcacccgccgggtgagcgagctgaaccagatgctgcttcagctgtttgaagaggctattctcgccctgaccgaggatgaaaaaccgcgcccgatagacgatgacttccagctgcgcggcacccttatcgatctgcgtgacgacacgctgtttattcgcgaaccgcaggccattctgcgcatgttttatatgatggtgcgcaacagcactatcaccggcatctactccacgacgttgcgccatctgcgccatgcccggcgccatctgacccagccgctgtgctatatcccggaggcgcgcacgctctttctcagcatgctgcgccatcagggggcggtcagccgcggactgctgccgatgcatcgccatagcgtgctgtgggcctatatgccgcagtggtcacatatcgtcggccagatgcagttcgatctgtttcacgcctacaccgtcgatgaacacaccatccgcgtgatgctgaagctggagagctttgccaaagaagaaacccgcagccgccacccgctgtgcgtggagctatggccgcgcttaacgcacccggagctgattttaatcgccgccctgttccacgacattgcgaaagggcgtggcggcgaccactcgatcctcggcgcgcaggatgtgctgaagtttgccgagctgcacggactgaactctcgcgaaacgcagttggtcgcctggctggtgcgtcaccatctgctgatgtcggtcaccgcccagcggcgcgacattcaggatccggaggtgattaagcagttcgccgaggaagtgcaaacggaaaatcgcctgcgctatctggtgtgcctgaccgtcgccgacatctgcgccaccaacgaaacgctgtggaacagctggaagcagagtctgctgcgcgaactctatttcgccaccgagaaacagctgcgtcggggcatgcaaagcaccccggatatgcgcgaacgggtgcgtcatcatcagctgcaggcgctggccctgctgcggatggacaatattaatgaagaggcgctgcatcagatctggaaccgctgccgcgccaactatttcgtgcggcatacgccgacgcagctcgcctggcacgcccgcaacctgctgcgtcacgatctgaataagccgatgattctgctgagttcgcaggccacccgcggcggtacggagtgacgtattttttttagtgaatggaaagaaaca

SEQ ID NO: 11 - DNA, 1410 bp, Artificial
  mutated ntrC
atgcaacgagggatagcctggatcgttgatgacgatagctccatccgctgggtgcttgaacgcgcgctcaccggagccggcttgagctgcacaacgttcgaaagcggcaatgaggtgctagatgccctcaccaccaaaaccccggatgtactgctgtcagctatccgtatgccgggaatggatggtctggcgctgctcaaacagattaagcagcgtcatccaatgcttccggtcatcataatgaccgcacattccgatctggacgctgcggtcagcgcttatcagcaaggcgcgtttgattatctgcccaaaccttttgatattgatgaagccgtcgccctggtcgaccgggcgataagccactatcaggagcagcaacagccgcgaaatgcgccaataagcagcccaactgccgacatcatcggcgaagcgccggcaatgcaggatgtctttcgcattattggccgtttgtcgcgatcatccatcagcgtgctgattaatggcgaatccggtaccggtaaagagctcgtcgctcacgccctgcatcgtcatagcccacgttcaaaagcgccgtttatcgcactgaatatggcggcaatacccaaagacctgattgagtccgagctgttcgggcatgaaaaaggggcctttaccggcgccaataccgtccgccagggacgcttcgaacaggctgacggcggcacgctattcctggatgaaattggcgatatgccgcttgatgtccagactcgtctgctgcgcgtgctggcggatggccagttttatcgcgtgggcggttacgcgccggtgaaggtcgatgtgcggatcatcgccgccacccaccagaacctggaacagcgcgtgcaggaggggaaattccgtgaagatttgttccaccgcctgaacgtgatccgggtgcatttaccgccgctgcgcgagcgccgggaagatattccacgcctggcccgccattttctgcagatagccgcccgcgagctcggtgttgaagccaaacagctgcatccggaaacggagacagcgctgacacgcctggcgtggcctggcaacgtccgtcagctggaaaacacctgtcgctggctcaccgtcatggccgccggccaggaggtactgacgcaggatctgccgagcgaactgtttgagactacggttccggacagcccgacgcagatgcagcccgacagctgggcgacgctgctgggtcagtgggccgatcgggcgttgcgatccggtcatcaaaacctgctctcagaagcgcaacccgaaatggagcgcacgctgctgacgaccgccctgcgccatacccaggggcacaagcaggaggctgcgcgtctgctgggatggggtcgtaataccctgacgcgtaagctaaaagagctgggaatggagtag

SEQ ID NO: 12 - protein, 9 aa, Artificial
  Artificial LAGLIDADG polypetide megenuclease family
LAGLIDADG

SEQ ID NO: 13 - DNA, 1997 bp, Artificial
  nifL::Prm5
atgaccctgaatatgatgatggatgccggcggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaacaactcacttcacaccccgaagggggaagttgcctgaccctacgattcccgctatttcattcactgaccggaggttcaaaatgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtcgctaacctggtcgcgcaaaccgtgcgtttgatggcaccaccggcagtgcgcccttccccgcgcgccgccataacacaggccgccagcccgaaatcctgcacggcctcacgcgcatttggttttgaaaatatggtcggtaacagtccggcgatgcgccagaccatggagattatccgtcaggtttcgcgctgggacaccaccgttctggtacgcggcgagagtggcaccggcaaggagctgattgccaacgccatccaccaccattcgccgcgtgccggtgcgccatttgtgaaattcaactgtgcggcgctgccggacacactgctggaaagcgaattgttcggtcacgagaaaggggcatttaccggcgcggtacgccagcgtaaaggccgttttgagctggccgatggcggcacgctgtttcttgacgagatcggcgagagtagcgcctcgtttcaggctaagctgctgcgcattttgcaggaaggcgaaatggaacgcgtcggcggcgacgagacattgcaagtgaatgtgcgcattattgccgcgacgaaccgcaatcttgaagatgaagtccggctggggcactttcgcgaagatctctattatcgcctgaatgtgatgcccatcgccctgccgccactacgcgaacgccaggaggacattgccgagctggcgcactttctggtgcgtaaaatcgcccataaccagagccgtacgctgcgcattagcgagggcgctatccgcctgctgatgagctacaactggcccggtaatgtgcgcgaactggaaaactgccttgagcgctcagcggtgatgtcggagaacggtctgatcgatcgggatgtgattttgtttaatcatcgcgaccagccagccaaaccgccagttatcagcgtctcgcatgatgataactggctcgataacaaccttgacgagcgccagcggctgattgcggcgctggaaaaagcgggatgggtacaagccaaagccgcgcgcttgctggggatgacgccgcgccaggtcgcctatcgtattcagacgatggatataaccctgccaaggctataa

SEQ ID NO: 14 - protein, 565 aa, Paraburkholderia tropic
MQKSGAGEARRELDAVYEVSKTLVSSLDVAQTFRTSLEYLLNALDWRRAFVVVAELDGELHGMCSAGLSRDEHRRIRFHPGEGIVGRVFKSSMPAVVPDVRDEPVFLDRTGSAGEPGEFGDARVALLATPIRVERNTLGVLAVFCINPGGTRMFAGDLDLMKIVSTLMAQALLLHRSVSAAHGRMQEEVRRMQKAIRPAPPLDHVVGVSEPMQRIFEQVHQVAPARTTVLLRGESGTGKEVIARAIHRLSPRNEQAFVSVNCAALTESLLESELFGHEKGAFTGAQGQRKGRFELAHGGTLFLDEIGDISPSFQAKLLRVLQEREFERVGGASAVKVDVRLILATNRNLERMVKEGEFRADLYYRINVVSIQLPPLRERREDIPAMAEYFLDRFNRDNSRALHFSTDAMRVLTSCYWPGNVRELENCVERTATMTHGDLIDRLAFLCQEDQCLTRVLHHIERDDAVRPARLADIPIREIPGMPGGAPRDADLPFALPDDLADDSVADGFHSGDAEEGKPEGERERLIWAMERCGWVQAKAARLLRITPRQIGYALHKYGIEVRRF

SEQ ID NO: 15 - protein, 627 aa, Azospirillum sp.
MPSAAQSASSSSLELLTIYEVSKILGSSLDLEQTLREVLRALSYQLQMHRGRVYLQGEDGALRLVAAQGLPKDSPAQEIDYNQGEGISGRILKTGMPAVVPNLAEEPLFNNRSGGRDDLDEQVASLVGVPIKAAGTVIGVLTIDRISDDGPSGHFGSDVRFLTMVANLIGQTVRLHRTVAEERRFMMRETFRVQKELRPVVAPVNDVVCTSPNMVDVLAQVHRVAPFKSTVLIRGESGTGKELIARAIHNLSPRKDAPFIRVNCAALPEALLESELFGHEKGSFTGAQKDHKGRFELASGGTLFLDEIGDISSNFQAKLLRVLQEQEFERVGGSKTIKTDVRLICATNLNLEEAVGHGKFRADLYFRINVVTIHLPPLRERRGDIATLARHFVAKFGRDNGLNLDIAAEALEVLNRCTWPGNVRELENCIERAATQCRNGTIRVPDLSCSMNLCNSSVLFQYRTMGAAVGGLAPSMSGAVTNPAQGRTQPAVNAAMPSPVPAPANGAAANGAPNGAQWPACASGCSAGPAPACGASKPLPPSAIVPLPAPLPASSAPAIPSAPAPQPVSAGPDLPLDEPESGPLRDRLVWAMERTGWVQAKAARLLGMTTRQVSYALRKYNIEIKRF

SEQ ID NO: 16 - DNA, 300 bp, Artificial
  acnB promoter
cgggcccggattgccttgcgacgcgcgcgacgaatccacctgaattcgtccgcacaacagaaaaatcccgctaaaacagcgatctatcgcaaatcgaacaaatcgcacgctcccgcccgccgccccacgcgcggcccctcatcgccaaccgctccgccgatcaactcttatataagacataagacatttgacgatagacggcacagcgattagaatcgcggtcaaagtagtggtagtgcccggaatagcctcggcgtgtctggaacgccttcccctgaaacgcaatacagcaggtccccc

SEQ ID NO: 17 - DNA, 292 bp, Artificial
  gapA1 promoter
cctggttcatcgcccggcgcacccgcgatgccgggctgctgtcggtggcggccggcgaggacaccattgccgccctgacggcggcgggcgtggatgaccgcttcagcgagttgaccagccgcgatgccctgccggcatgactggatgcggtgtcgccgcgggcggtcccggcgtgagcgagggcccacgccttaaaattgcaattcctgcgggtcgatccccgctttccccggttgaaacaggtctccgccccaatcggcacaggaccgcaaccgaacgactggagcacatc

SEQ ID NO: 18 - DNA, 305 bp, Artificial
  groS promoter
tcctctttctgctgtcccggcgcggggcgccgctgccgggcaggctggtccttcgtgaccgtttgaagaaatcttgcctgtgcccgccctcgggtgcaacgggaaatatcaagttatttcagtgtgttagcagcaggtttgggtgagtgctgccggcatttgcggcaaatttcgaaacgagcctcttgaatcccaaaaccgcttcgattagctgtctggcactcgccgggagggagtgctaacagcaaccagcccgcgccaattcacgtcagtcagaaccaagccttgatccaaggaggcattcc

SEQ ID NO: 19 - DNA, 304 bp, Artificial
  ompA promoter
cagcttggaaatgcggcgctgcgtggcgcaccgccgcgcactccattccactccaaaagggtagctctttagcgcaaaaattggggcaactgcgcacaagcaccatccaataaagcgcaaagtatttaatatatgaatttacatcatccggggctccgtgttttccccgccactgttcttggaatgaggtgacttccattctcgcggccacttgtccgcatactgggggcgggactaaatcagggtgcaccagacaccgctcgcgccccggcctgacgggacgcatgcaccgaggatgacaccc

SEQ ID NO: 20 - DNA, 340 bp, Artificial
  oprF promoter
tcaacctgcgcatggaccgcccgaatgccgaggagacggtcgcctgcgtcctctcccacgacgagatcggcgtgcggataccggaggcgttcaagagcgaggatctgcagcccattcccggcgccgccctggacgacgtcatcaacgccttcgcgacggccaagggcagcgatgcccgcgccaagcggctgaccgtgaaggtgttgcccggcacgctggcgcagggggagtgacgccggaaaaactggagccgactgttggccggctcacagcggggaaccgggggatggcgatagaaccgggggcaacgccgttccttctcctgcccatggagcctcag

SEQ ID NO: 21 - DNA, 320 bp, Artificial
  pflB promoter
gctgcgaacgaccgattccgtcgcgcggccgacgcgccctttccaagcatttccaaccgttccacgcctaccctgcgacaaaatgccgggtcggttcgccttgtccggaaggccgcccgggagtatgtagtaaaactacagagcgggcgcaacgcacggcatcagccactccgcgcccggcctgaaaatatgacgtgtatcaacgaattcggcgccgatcgcgcctatgcttccaatcgtcccgcgcggttcgcgggcaccacgagaccgccgacacggcattcgacccgcgccgacggctatatctctgaggagtcatc

SEQ ID NO: 22 - DNA, 331 bp, Artificial
  pgk2 promoter
gtctcgcggttgatctcggcctcagccatcgcctgcgcttcaacccccgttttgcggcccaatggcctacagttgggtgtttgcacagcccatggacagggtcaagttaccgaaatgttgcagaagcctgcggttgcgatgcccccgtccccgtatcccggcctgttccggagcaaccggaacgacgcatcgaacatccggttgcagggtcattatgccccatttcaccccacccctggggcgttgcaaagcgtgcaggcctgtggttgggttcccgcaaactgaaacggcccgtcccgctgggatcggccataattggggaagtgtgacg

SEQ ID NO: 23 - DNA, 304 bp, Artificial
  ppsA promoter
ctgacacaatttctgtagtcggcacggtagaatagcggcaacctgttggataagcaattgcagtgcaaccacgccaaacggcagtccatcgtggctcaatctggcgtcaacttgcagcgattttgcataactccctgcaatcgcgcgcatcttcctgcttttccttccgctgcgtgtgagctttcgcacctctcgcgattgcttctccaacaggttgcgcaagacgcgaatccgcttccaatgggcggtccgcgttcgagcccacttgcgcaatcgtgaatttttttcacacttaggggcttgt

SEQ ID NO: 24 - DNA, 263 bp, Artificial
  rpmB promoter
atttcgtggtggccggaacacgtctctactccttcgccgaccacgggaagatgtagcaccggaccgcgcagaccggccgcaaacccgcacgggaggcgggttctgcgtcgacaaggcaggctcgggaaagcgtgacgaagaaaattgttaaatgcatttttctaaccgcgacacaattgtttttcgcaagtcattgaaaagtctaagttttttccctatactctcgcttttcccaaattcggaaatttttaaggagtctcaca

SEQ ID NO: 25 - DNA, 325 bp, Artificial
  rpmBC promoter
ttgtggggactgcgtttttgtgttcgattttcaggagtcggaggcaggatgcgggacccagaagccaaagaaaaacgtcttgttcagggtgagtgccagaacggcatgaaggacatccagacgcacgacgcttctctttgtcttctgaagcgactgcagaaggcaagtttggtcgggcagcgggtaagttatgacaagcccaaatccgctgccgtcagccagcggttggtagaggccaaccaccaagcttccaaggctcgcgaagccatcgggtctcagtcggtgaacactcgggtcgtcctacccgccgtgattcggagatcgt

SEQ ID NO: 26 - DNA, 110 bp, Artificial
  tufA-2 promoter
gccggtatctagcaggcctccagaggcttcgcatcgagcaggcagcagcaacccagtgattgtttgcgcgccccgcgcaacgtacggaaaaatcctcttaggagtcgaaa

SEQ ID NO: 27 - DNA, 304 bp, Artificial
  cspA3 promoter
gcgacccggtctaaccctttatttcggcaagaagatcctgttcatgccgggaatccgccaagaaacgggctaatccccttcgacaaggtaccgtcgccttggtcgtaactccactccgaacttggccgtcgttagtcctatgccgtgagatggaattgcccgcaaccacagcatgcggctagcctccccgcacaatgtcttggttccgcttgcccggcagaccttacggcgcggccgatgtcggcgatcccggacctatccgtttggctaaagaccagtctcgcagtgactaaggggggaggcc

SEQ ID NO: 28 - DNA, 338 bp, Artificial
  cspA5 promoter
gaggaaatcgtcgaagccaatccgccgcccccgccgcgcgtacagcagcaacaggccgccggctgacgccggcataacgctccggcagttcggcttttcttttcgttcgcccccacacgcccggccgtctttcaccttgaaagccggaccgggcgttcccatgtctgaagggtcattctgcttcggactgcggggcgttcaaccgcccccacgctcagccccccttttttcgccattgcgagcattcgttcgcgagcttatgggggccgccaatcccacacccagtctgatgcgccgcaccggtggtgtggcgccctttgcgttatgggagacaagac

SEQ ID NO: 29 - DNA, 250 bp, Artificial
  cspD2 promoter
agacaagggattgcgggcggctttctgcaccatcgggcggcattgagcgaaatctaaagcacccgaaaaagtcattaaatatttaaattgaagtttcgtgccgaaaatctcttgcattctcgatttaatgagtataatcgtcgcgtcgttaggattcgagctggtagtgcggtcatctcgctgtcattcctcccgagtaacgataaagagccgggcgcaagccccaatcaattaaatttaggagtccgta

SEQ ID NO: 30 - DNA, 309 bp, Artificial
  cspJ promoter
gtaatttgcaacgcgatgaacagacgcgatttggtggcattgtgatgcggctttgtttatgaaaccgaaccatcgatgcggacacgctttcgctttccctaactcgcgcattgcgagtatcggcccgtcgccaagtgatgccgcccatcatcggagcgttacagggcatcaccgcactggtcattttctggaattttatgataagctcacggacgccggggccttgttcttggtcatacagcggtgccgcgcccagttcagcgtcagggccgcccctcccccggctacaggacgtgaaagggacctgtc

SEQ ID NO: 31 - DNA, 239 bp, Artificial
  gltA1 promoter
cgaaaagctgggcaaggtggcacagccgctgcgcgccgcgctgaccggttcgacggtgtcgccaccgatcttcgaggtcgttgaactgctgggtcgggaagcgacgctttctcgcatccgcgatgcagcgggtgcagcataatggtcatttccgtgcgttgccattgggcagccttggaactatgctgcgccgacgaaaaaagggcaccaagcccgaccctcaacgataaggacgtgcc

SEQ ID NO: 32 - DNA, 248 bp, Artificial
  gltA2 promoter
agggggtgcgtgtttgaggcgtgtcggtcatgatgtcctctgctatcttctgagagagtcaacgaccaggaaggatgcgtgttccgcatccttcctggtcgtagggagaccgggagaagacggcgcggcgctccagggcgccgaagagcgtgctttcaagcgcgcctgacgacgggccaaccaccaaggtccggcagcggaggccgcgagccgccgcccagccggccagcaaggagtgtgaaaaggtc

SEQ ID NO: 33 - DNA, 341 bp, Artificial
  rpsF promoter
gtgcaatccttgtgtcaacggcaacatgcatttccagcggtcgaaaaagcggcttttgcggtttttcttcacttatgttcacttatggatagatgggcagctggccctctatatatagaggggttgaaaatggtgagaggatcgctctcttcctgcgccccgcgccttcctgcgtcttcttgtccttcaatgaaggtgatttgtctctgccaggcgttggtcggacctgccgatgattgctgttctatccaaaagcttgtatgatcttgggttttcctcttcccacacccgtcttgacgccggggtgggcgtcttcaataagtccataaggagtctacatg

SEQ ID NO: 34 - DNA, 268 bp, Artificial
  rpsL promoter
ccttttggctcccgcgccggctctgcgggaccgttcgcaagctgaaataccgtccttgactctcccgcggcctttccctaaagtccgcgaacttcaaaggacgccggggctctccgtgcgtccgcttgaatccaccgatcccgccgatcggagtcctccggtcggctttcgctcattcagcccgctcggaacggcaacgtttcgcaggcgggcttttgcttcgtccggctcgtccggccgagtgatcttgaagggatgaagggcagat

SEQ ID NO: 35 - DNA, 246 bp, Artificial
  rplL promoter
agattctggacgcggagggggtcaaggccctcgcctcgctcccgtcgctggacgaactgcgtggccgcctcgtgggcatgatccagaccccggcgacccgtatcgccggtgttctccaggctccgggcggccaagtcgcccgcgtgcttgctgcctacgcgaagaaggacgacgaggcggcggcctaaccaggcctagtcactcgttttttacatccccagaactcaagtcgtttggagtagtaac

SEQ ID NO: 36 - DNA, 312 bp, Artificial
  rplM promoter
tggcgagatcctgctgttctccgaaggcgatccggaaccggtcgcccatgtcaccggcacctactcgatccctccgcacgaccccgtcacggttgcggtatctcattaccgtactggcgaagggtcataaaaaagcgtttcagatcaatgggtttgcgatcgcggtatcaaattaccgcgcccggaaaccattgataataaacgataatttttccgttgacagagccctgccttgccccgtagaaagccgtccgcttcggcgccccggtatcggcggccagccgatccgtattcgaagaaacgagtgtggcg

SEQ ID NO: 37 - protein, 572 aa, Paraburkholderia xenovorans
MLNIQATVARQALDSVYEVSKTLVSSLDVAKTFRESLNYLLHTMEWRRAFVVLAEPDGQLRGLCAAGLSRDEQQRLQFRPGEGIVGRAFKSGIGVIVPDVNDEPVFLNRTGGADQMPGACIAMLATPIHADRRTLGVLAVDCVNPGASRLFADDLHLMKIVATLMGQALLLQRSVSAAHDSMQDEVRRMQKAIKPSQQIDQVVGVSAAMQAVFGQVRQVAPARTTVLLRGESGTGKEVIARAIHNLSPRSGDSFVRVNCAALTESLLESELFGHEKGAFTGAQGQRKGRFELAHGGTLFLDEIGDISPSFQAKLLRVLQEREFERVGGTTPVKVDVRLILATNRNLERMVKAGEFRADLYYRINVVSIELPPLRERREDIPAMAQHFLARFNRENAQSIRFDAEAMRVLTNCYWPGNVRELENCVERTATMTRDGVIDRFKFLCQEDRCLTKVLHYIEREDAVRPARLADIPIAEVSSSQPPYGHHDLDAPSAPAAWSGGTDPDAETELDPAADERAFAAASAGKPEGERERLIWAMERCGWVQAKAARLLGITPRQMGYALHKYAIEVRRF

SEQ ID NO: 38 - protein, 570 aa, Paraburkolderhia aromaticivorans
MVNIQATVARQALDCVYEVSKTLVSSLDVAKTFRESLNYLLHTMEWRRAFVVLAESDGQLRGLCAAGLSRDEQQRLQFRPGEGIVGRAFKSGIGVIVPDVNDEPVFLNRTGGADQMPGACIAMLATPIYADRRTLGVLAVDCVNPGASRLFADDLHLMKIVATLMGQALLLQRSVSAAHDSMQDEVRRMQKAIKPSQQIDQVVGVSAAMQAVFGQVRQVAPARTTVLLRGESGTGKEVIARAIHNLSPRSGDSFVRVNCAALTESLLESELFGHEKGAFTGAQGQRKGRFELAHGGTLFLDEIGDISPSFQAKLLRVLQEREFERVGGTTPVKVDVRLILATNRNLERMVKAGEFRADLYYRINVVSIELPPLRERREDIPAMAQHFLARFNRENAQSIRFDAEAMRVLTNCYWPGNVRELENCVERTATMTRDGVIDRFKFLCQEDRCLTKVLHYIEREDAVRPARLADIPIAEVSSPPYGHHDLDVPSVPAAWSGGTDTDAETELDPATDERAFATSSAGKPEGERERLIWAMERCGWVQAKAARLLGITPRQMGYALHKYAIEVRRF

SEQ ID NO: 39 - protein, 577 aa, Paraburkholderia kururiensis
MQNPGAGEARRELDAVYEVSKTLVSSLDVAQTFRTSLQYLLHALEWRRAFVVVAEPGGQLRGLCSAGLSSDEHQRIRFHPGEGIVGRVYKSGLPAVVPDVRDEPVFLDRTGSADVPGEFGDARIALLATPLRQEHHTLGVLAVFCMNPGGTRMFAGDLHLMKIVSTLMGQALLLHRSVSAAHDRMQEEVRRMQKAIRPFQPLDQVVGVSDPMRRIFEQVHQVAPARTTVLLRGESGTGKEVIARAIHRLSPRGEQAFVSVNCAALTESLLESELFGHEKGAFTGAQGQRKGRFELAHGGTLFLDEIGDISPSFQAKLLRVLQEREFERVGGSAPVKVDVRLILATNRNLERMVKEGEFRADLYYRINVVSIQLPPLRERREDIPEMAQYFLDRFNRDNGRELRFSPDAMRVLTSCYWPGNVRELENCVERTATMTHSDVIDRLAFLCQEDQCLTRVLHHIEREDAVRPARLADIPIREIPGTPASDAGTGAREHDSSFALPDDFGDDVSTDPNTGEPSGACHPADTDEGKPEGERERLIWAMERCGWVQAKAARLLRITPRQIGYALHKYGIEVRRF

SEQ ID NO: 40 - protein, 547 aa, Paraburkholderia phymatum
MPHFHLNDSLPDFDVVYEVVKTLVSSRDAERTLDKSLRYLSYALGWRSAFIAVTEPDGHLCSLCSTGLSEGWRDRARFLSGEGIVGRLHSSDAAVVVPELNEEPLFADDIGAVGGSEGEHVALLGTPIRHEGRPLGVLVAFCENPDGKRTFGDDLQLMKIVAAPMAQALLLHRDEKAMHDGAEPVRRPRKETIRAYQIDNTIGASAAMQQVFAQVHQVAPARTTVLLRGESGTGKELIARSICRLSPRKEQPFIAVNCAALTETLLESELFGHEKGAFTGAQSQRKGRFELAHGGTLFLDEIGDISSSFQAKLLRVLQEREFERVGGATPVRVDVRLIVATNRNLERMVRDGEFRADLYYRINVVSILLPPLRERREDIPAMAQYFLDRFNRDNGRLLRFSDEALRVLSNCYWPGNVRELENCVERTATMTHHDIIDRLSFLCEIDQCLTKVLHHIEREDAVRPGPSSNLVASEAPNALASGHAADLNGGRFDVPGGDGRPEGERERLVWAMERCGWVQAKAARLLGITPRQIGYALRKHEIEVRRF

SEQ ID NO: 41 - protein, 547 aa, Paraburkholderia phenoliruptrix
MPHFHLNDSLPDFDVVYEVVKTLVSSRDAERTLDKSLRYLSYALGWRSAFIAVTEPDGHLCSLCSTGLSEGWRDRARFLSGEGIVGRLHSSDAAVVVPELNEEPLFADDIGAVGGSEGEHVALLGTPIRHEGRPLGVLVAFCENPDGKRTFGDDLQLMKIVAAPMAQALLLHRDEKAMHDGAEPVRRPRKETIRAYQLDNTIGASAAMQQVFAQVHQVAPARTTVLLRGESGTGKELIARSICRLSPRKEQPFIAVNCAALTETLLESELFGHEKGAFTGAQSQRKGRFELAHGGTLFLDEIGDISSSFQAKLLRVLQEREFERVGGATPVRVDVRLIVATNRNLERMVRDGEFRADLYYRINVVSILLPPLRERREDIPAMAQYFLDRFNRDNGRLLRFSDEALRVLSNCYWPGNVRELENCVERTATMTHHDIIDRLSFLCEIDQCLTKVLHHIEREDAVRPGPSSNLVASEAPNALASGHAADLNGGRFDVPGGDGRPEGERERLVWAMERCGWVQAKAARLLGITPRQIGYALRKHEIEVRRF

SEQ ID NO: 42 - protein, 616 aa, Azospirillum oleiclasticum
MRVVSRQSSSNLELLAIYEVSKILGSSLDLQQTLREVLRVLSYHLQMRRGRVYLLGDDNVLRLVAVNGFSNEQITATAISEGEGISDRILKTGMPVVIPNLAEEPLFRNVAGGREDLDEQIASLVGVPIKAAGSVVGVLTIDRISDEGPTTHFGSDVRFLSMVANLIGQTVRLHRTVAEERRYMMRETFRVQKELRQPEPIADIVGGSPSMMEVLAQVHRVAPFKSTVLIRGESGTGKELIARAIHNLSQRKDGPFIRVNCAALPESLLESELFGHEKGAFTGAQKDHKGRFELASGGTLFLDEIGDISSNFQAKLLRVLQEQEFERIGGTKTIRTDVRMVCATNANLEEAVGQGKFRADLYFRINVISIMLPPLRERPGDIATLARHFVTKFSKDNGLNIKLADDAVGVLDRCPWPGNVRELENCVERAATQCTNGVIKAVDLSCSMNLCNSSILFQYRTTGTPVGGLAPAAAVTPTGPRPAAPNGHNGGAPPFSAPPFSVQSSPLPAPAASPNGKPWPACASACAASPTCGSGTSSVTVPLAPLPEPEPADGAEADADADALSDVPDNGSLRERLLWAMDRTGWVQAKAARLLGMTTRQVSYALRKYNIEIKRF

SEQ ID NO: 43 - protein, 603 aa, Azospirillum halopraeferens
MPSTTRSAASSNLELMAIYEVSKVLGSSLDLQQTLREVLRVLAYQLQMHRGRVWLLGDDQVLRLVAVNGAPVDEARQVELREGEGIIGRILKTGMPAVVPNLAEEPLFLNRTGGRDDLDEVVASLVGVPIKVAGSAVGVLTIDRISDDGPTTHFGSDVRFLTMVANLIGQTVRLHRTVAEERRYMMRETFRLQKELRAPEPIADIVCSSPNMVDVLAQVHRVAPFKSSVLIRGESGTGKELIARAIHNLSPRKDAPFIRVNCAALPESLLESELFGHEKGAFTGAQKDHKGRFELASGGTLFLDEIGDISSNFQAKLLRVLQEQEFERIGGTKTIKTDVRLICATNLNLEEAVGLGKFRADLYFRINVVSIFLPPLRERRSDIAPLARHFVAKFAKDNGLTLALETEAQEVLSRCPWPGNVRELENCIERAATQCRDGTIRAVDLSCSQNLCNSSILLQYRTAGKAVGGLAPAVHGPGAIVPGAASHVAPPTGCNTCGAAPGQPCQAPAPAGNAAADAAPAGPAGPAAAGPAAPPASALPPDPDGDGPVDLPDEPDSGSLRERLIWAMDRTGWVQAKAARLLGMTTRQVSYALRKYNIEIKRF

SEQ ID NO: 44 - protein, 557 aa, Azospirillum formosense
MPGAMRQSTSNLELLTIYEVSKILGSSLDLQQTLREVLRALAYQLQMHRGRVYLVGEDNVLRLVAANGLSNEAAAQIEFRDGEGITGRILKTGMPAVVPNLAEEPLFLNRTGGREDLDEQVASLVGVPIKAAGVVVGVLTIDRISDEGPQGHFGSDVRFLTMVANLIGQTVRLHRTVAEERRFMMRETFRMQKELRPIAAPINDVVCTSPNMLEVMAQVHRVAPFKSTVLIRGESGTGKELIARAIHNMSPRKDAPFIRVNCAALPESLLESELFGHEKGAFTGAQKDHKGRFELASGGTLFLDEIGDISPNFQAKLLRVLQEQEFERVGGSKTIKTDVRLICATNLNLEEAVGHGKFRADLYFRINVVTIHLPPLRERRQDIGPLARHFVAKFAKDNGMNLVMEDEALEVLNRCTWPGNVRELENCIERAATQSRDGIIRTESLSCSLNLCNSSVLFQYRTLGASVGGLAPSMGPGSVNRVPPGRPGVPAPANAPKTPAMPAPVPEPAGAGAAGPARAPGPCPRPSPLRGPPPPPGRGSPLSLPPPPPRVPPPPPP

SEQ ID NO: 45 - protein, 625 aa, Azospirillum brasilense
MPGAMRQSTSNLELLTIYEVSKILGSSLDLQQTLREVLRALAYQLQMHRGRVYLVGEDNVLRLVAANGLSNEAAAQIEFRDGEGITGRILKTGMPAVVPNLAEEPLFLNRTGGREDLDEQVASLVGVPIKAAGVVVGVLTIDRISDEGPQGHFGSDVRFLTMVANLIGQTVRLHRTVAEERRFMMRETFRMQKELRPVAAPINDVVCTSPNMLEVMAQVHRVAPFKSTVLIRGESGTGKELIARAIHNMSPRKDAPFIRVNCAALPESLLESELFGHEKGAFTGAQKDHKGRFELASGGTLFLDEIGDISPNFQAKLLRVLQEQEFERVGGSKTIKTDVRLICATNLNLEEAVGHGKFRADLYFRINVVTIHLPPLRERRQDIGPLARHFVAKFAKDNGMALVMEDEALEVLNRCTWPGNVRELENCIERAATQSRDGIIRTESLSCSLNLCNSSVLFQYRTLGASVGGLAPSMGPGAINRVPPGRPGGPAAANAPKTPAMPAPVPEPAGAGGGAWPACASGCSAGPSPVCGAAQPVVPVPLIPLPLPEPSAPAAAAPPPSPAAAPPPAAEVPLDEPESGSLRDRLLWAMERTGWVQAKAARLLGMTTRQVSYALRKYNIEIKRF

SEQ ID NO: 46 - protein, 624 aa, Azospirillum baldaniorum
MPGAMRQSTSNLELLTIYEVSKILGSSLDLQQTLREVLRALAYQLQMHRGRVYLVGEDNVLRLVAANGLSNEAAAQIEFRDGEGITGRILKTGMPAVVPNLAEEPLFLNRTGGREDLDEQVASLVGVPIKAAGVVVGVLTIDRISDEGPQGHFGSDVRFLTMVANLIGQTVRLHRTVAEERRFMMRETFRMQKELRPIAAPINDVVCTSPNMLEVMAQVHRVAPFKSTVLIRGESGTGKELIARAIHNMSPRKDAPFIRVNCAALPESLLESELFGHEKGAFTGAQKDHKGRFELASGGTLFLDEIGDISPNFQAKLLRVLQEQEFERVGGSKTIKTDVRLICATNLNLEEAVGHGKFRADLYFRINVVTIHLPPLRERRQDIGPLARHFVAKFAKDNGMALVMEDEALEVLNRCTWPGNVRELENCIERAATQSRDGIIRTESLSCSLNLCNSSVLFQYRTLGASVGGLAPSMGPGAVNRAPPGRPGVPAPANAPKMPAPVPEPVGAGGGAWPACASGCSAGPSPVCGAAQPVVPVPLIPLPLPEPSAPAAAAPPAASVANAAPPPAAEVPLDEPESGSLRDRLLWAMERTGWVQAKAARLLGMTTRQVSYALRKYNIEIKRF

SEQ ID NO: 47 - protein, 619 aa, Azospirillum doebereinerae
MPSAARSIQPASSSSLELLTIYEVSKILGSSLDLEQTLREVLRALSYQLQMQRGRVYLQGEDNTLRLVAAHGLSKDALAQEIDVQAGEGITGRILKTAMPVVIPNLAEEPLFLNRSGGRDDLDEQVASLVGVPIKAAGTVVGVLTIDRISDEGTGGHFGSDVRFLTMVANLIGQTVRLHRTVAEERRFMMRETFRVQKELRPVVAPVNDVVCTSSNMVDVLAQVHRVAPFKSTVLIRGESGTGKELIARAIHNMSPRKDAPFIRVNCAALPESLLESELFGHEKGSFTGAQKDHKGRFELASGGTLFLDEIGDISSNFQGKLLRVLQEQEFERVGGSKTIKTDVRLICATNLNLEEAVGHGKFRADLYFRINVVTIHLPPLRERRGDIALLARHFVAKFGKDNGLNLGIDGEALEVLNRCTWPGNVRELENCIERAATQCRNGTIRLPDLSCSMNLCNSSVLFQYRTMGAAVGGLAPSTSNTPTNPNIARAPQPASMPSAVPAPAVNVAWPSCASGCAASAPPGCSAVKPVPMPPSAILPLPTPEPAGSSPSAPSAPAPQPGSADLPVDEPDSGPLRDRLLWAMERTGWVQAKAARLLGMTTRQVSYALRKYNIEIKRF

SEQ ID NO: 48 - protein, 638 aa, Azospirillum oryzae
MPSAAQSASSSSLELLTIYEVSKILGSSLDLEQTLREVLRALSYQLQMHRGRVYLQGEDGALRLVAAQGLPKDSPAQEIDYNQGEGISGRILKTGMPAVVPNLAEEPLFNNRSGGRDDLDEQVASLVGVPIKAAGTVIGVLTIDRISDDGPSGHFGSDVRFLTMVANLIGQTVRLHRTVAEERRFMMRETFRVQKELRPVVAPVNDVVCTSPNMVDVLAQVHRVAPFKSTVLIRGESGTGKELIARAIHNLSPRKDAPFIRVNCAALPESLLESELFGHEKGSFTGAQKDHKGRFELASGGTLFLDEIGDISSNFQAKLLRVLQEQEFERVGGSKTIKTDVRLICATNLNLEEAVGHGKFRADLYFRINVVTIHLPPLRERRGDIATLAKHFVAKFGRDNGLNLDIAAEALEVLNRCTWPGNVRELENCIERAATQCRNGTIRVPDLSCSMNLCNSSVLFQYRTMGAAVGGLAPSMSGTPTNPAAGNPGVGNPAQGRAQPAPMNGAAMPPAVPAPANGALNGAPGGAQWPACASGCGAGPMPVCGASKPLPPSAIVPLPAPQPAASTPAIPSAPAPQPAAAGPDLPLDEPESGPLRDRLVWAMERTGWVQAKAARLLGMTTRQVSYALRKYNIEIKRF

SEQ ID NO: 49 - protein, 560 aa, Azospirillum melinis
MPSAAQSASSSSLELLTIYEVSKILGSSLDLEQTLREVLRALSYQLQMHRGRVYLQGEDGALRLVAAQGLPKDSPAQEIDYNQGEGISGRILKTGMPAVVPNLAEEPLFNNRSGGRDDLDEQVASLVGVPIKAAGAVIGVLTIDRISDDGPSGHFGSDVRFLTMVANLIGQTVRLHRTVAEERRFMMRETFRVQKELRPVVAPVNDVVCTSPNMVDVLAQVHRVAPFKSTVLIRGESGTGKELIARAIHNLSPRKDAPFIRVNCAALPESLLESELFGHEKGSFTGAQKDHKGRFELASGGTLFLDEIGDISSNFQAKLLRVLQEQEFERVGGSKTIKTDVRLICATNLNLEEAVGHGKFRADLYFRINVVTIHLPPLRERRGDIAMLAKHFVGKFGRDNGLNLDIAPDALEVLNRCTWPGNVRELENCIERAATQCRNGTIRVPDLSCSMNLCNSSVLFQYRTMGAAVGGLAPSMSGTPTNPSVAPSAPGRPQTPAMPPAMPAPANGTAHGAANGAPNGAQWPACASGCGAGPMPVCGASKPLPPSAIVPLPTPQPAAW

SEQ ID NO: 50 - protein, 632 aa, Azospirillum palustre
MPSAAQSASSSSLELLTIYEVSKILGSSLDLEQTLREVLRALSYQLQMHRGRVYLQGEDGALRLVAAQGLPKDSPAQEIDYNQGEGISGRILKTGMPAVVPNLAEEPLFNNRSGGRDDLDEQVASLVGVPIKAAGAVIGVLTIDRISDDGPSGHFGSDVRFLTMVANLIGQTVRLHRTVAEERRFMMRETFRVQKELRPVVAPVNDVVCTSPNMVDVLAQVHRVAPFKSTVLIRGESGTGKELIARAIHNLSPRKDAPFIRVNCAALPESLLESELFGHEKGSFTGAQKDHKGRFELASGGTLFLDEIGDISSNFQAKLLRVLQEQEFERVGGSKTIKTDVRLICATNLNLEEAVGHGKFRADLYFRINVVTIHLPPLRERRGDIAMLAKHFVGKFGRDNGLNLDIAQDALEVLNRCTWPGNVRELENCIERAATQCRNGTIRVPDLSCSMNLCNSSVLFQYRTMGAAVGGLAPSMSGTPTNPSVAPPAPGRPQTPAMPPAMPAPANGAAHGAANGAPNGAQWPACASGCGAGPMPVCGASKPLPPSAIVPLPTPQPAASAPAVPSAPAPQPASAGPDLPLDEPESGPLRDRLIWAMERTGWVQAKAARLLGMTTRQVSYALRKYNIEIKRF

SEQ ID NO: 51 - protein, 628 aa, Azospirillum ramasamyi
MPSSAQSASSSSLELLTIYEVSKILGSSLDLEQTLREVLRALSYQLQMHRGRVYLQGEDNALRLVAALGLPKDSTAQEIDYSQGEGISGRILKTGMPAVVPNLAEEPLFNNRSGGRDDLDEQVASLVGVPIKAAGTVVGVLTIDRISDDGPRGHFGSDVRFLTMVANLIGQTVRLHRTVAEERRFMMRETFRVQKELRPVVAPVNDVVCTSPNMVDVLAQVHRVAPFKSTVLIRGESGTGKELIARAIHNLSPRKDAPFIRVNCAALPESLLESELFGHEKGSFTGAQKDHKGRFELASGGTLFLDEIGDISSNFQAKLLRVLQEQEFERVGGSKTIKTDVRLICATNLNLEEAVGHGKFRADLYFRINVVTIHLPPLRERRGDIAMLAKHFVGKFARDNGLNLDIDRDALEVLNRCTWPGNVRELENCIERAATQCRNGTIRVPDLSCSMNLCNSSVLFQYRTMGAAVGGLAPSMSGTPTNPSGANPATGRPQPATGAGMPPSVPTAGAPNAAPNGAQWPACASGCSANPAPVCGASKPLPPTAIAPLPTPQPAAAAPAIPSAPAPQPADVPDLPLDEPESGPLRDRLVWAMERTGWVQAKAARLLGMTTRQVSYALRKYNIEIKRF

SEQ ID NO: 52 - protein, 621 aa, Azospirillum humicireducens
MPRAAQSASSSSLELLTIYEVSKILGSSLDLEQTLREVLRALSYQLQMHRGRVYLQGEDNALRLVAALGLPRESTAQEIDYSQGEGISGRILKTGMPAVVPNLAEEPLFNNRSGGRDDLDEQVASLVGVPIKAAGTVVGVLTIDRISDDGPSGHFGSDVRFLTMVANLIGQTVRLHRTVAEERRFMMRETFRVQKELRPVVAPVNDVVCTSPNMVDVLAQVHRVAPFKSTVLIRGESGTGKELIARAIHNLSPRKDAPFIRVNCAALPESLLESELFGHEKGSFTGAQKDHKGRFELASGGTLFLDEIGDISSNFQAKLLRVLQEQEFERVGGSKTIKTDVRLICATNLNLEEAVGHGKFRADLYFRINVVTIHLPPLRERRGDIAMLAKHFIAKFARDNGLTLDIDHDALEVLNRCTWPGNVRELENCIERAATQCRNGTIRVPDLSCSMNLCNSSVLFQYRTMGAAVGGLAPSMSGTATNPALGRPQQPAIAGMPPAVPAPANGAANGTQWPACASGCSAGPAPVCGASKPLPPTAIAPLPAPQPAAVAPAVPSAPAPQPADTPDLPLDEPESGPLRDRLVWAMERTGWVQAKAARLLGMTTRQVSYALRKYNIEIKRF

SEQ ID NO: 53 - protein, 218 aa, Azospirillum lipoferum - CI1666
LELLTIYEVSKILGSSLDLEQTLREVLRALSYQLQMHRGRVYLQGEDGALRLVAAQGLPKDSPAQEIDYNQGEGISGRILKTGMPAVVPNLAEEPLFNNRSGGRDDLDEQVASLVGVPIKAAGTVIGVLTIDRISDDGPSGHFGSDVRFLTMVANLIGQTVRLHRTVAEERRFMMRETFRVQKELRPVVAPVNDVVCTSPNMVDVLAQVHRVAPFKST

SEQ ID NO: 54 - protein, 215 aa, Paraburkholderia xenovorans - CI3044
QALDSVYEVSKTLVSSLDVAKTFRESLNYLLHTMEWRRAFVVLAEPDGQLRGLCAAGLSRDEQQRLQFRPGEGIVGRAFKSGIGVIVPDVNDEPVFLNRTGGADQMPGACIAMLATPIHADRRTLGVLAVDCVNPGASRLFADDLHLMKIVATLMGQALLLQRSVSAAHDSMQDEVRRMQKAIKPSQQIDQVVGVSAAMQAVFGQVRQVAPARTT

SEQ ID NO: 55 - protein, 227 aa, Rhodobacter capsulatus (strain ATCC BAA-309) - D5ARW9
LALDALYEIAKTFAAAPDPVAEVPQIFNVLSSFLDLRHGVLALLAEPGEGAGVNPYVIAATAFQRSPEAPAADVLPDAVARIVFRSGVPFVSFDLAAEFGAEAVPKRLRDAGQTLIAVPLRDPERSHFVLGVLAAYRSHDHNRSGFSDADVRVLTMVASLLEQALRFRRRIARDRERALEDTRRMLQTVTEQRGPAAPVSLDGIVGSSPAIAEVVAQIKRVASTRMP

SEQ ID NO: 56 - protein, 227 aa, Rhodobacter capsulatus (Rhodopseudomonas capsulata) P0CY94
LALDALYEIAKTFAAAPDPVAEVPQIFNVLSSFLDLRHGVLALLAEPGEGAGVNPYVIAATAFQRSPEAPAADVLPDAVARIVFRSGVPFVSFDLVAEFGAEAVPKRLRDAGQTLIAVPLRDPERSHFVLGVLAAYRSHDHNRSGFSDADVRVLTMVASLLEQALRFRRRIARDRERALEDTRRMLQTVTEQRGPAAPVSLDGIVGSSPAIAEVVAQIKRVASTRMP

SEQ ID NO: 57 - protein, 217 aa, Azospirillum brasilense - P30667
LELLTIYEVSKILGSSLDLQQTLREVLRALAYQLQMHRGRVYLVGEDNVLRLVAANGLSNEAAAQIEFRDGEGITGRILKTGMPAVVPNLAEEPLFLNRTGGREDLDEQVASLVGVPIKAAGVVVGVLTIDRISDEGPQGHFGSDVRFLTMVANLIGQTVRLHRTVAEERRFMMRETFRMQKELRPVAAPINDVVCTSPNMLEVMAQVHRVAPFKST

SEQ ID NO: 58 - protein, 229 aa, Azorhizobium caulinodans (strain ATCC 43989 ) - P09133
VALIGVYEISKILTAPRRLEVTLANVVNVLSSMLQMRHGMICILDSEGDPDMVATTGWTPEMAGQIRAHVPQKAIDQIVATQMPLVVQDVTADPLFAGHEDLFGPPEEATVSFIGVPIKADHHVMGTLSIDRIWDGTARFRFDEDVRFLTMVANLVGQTVRLHKLVASDRDRLIAQTHRLEKALREEKSGAEPEVAEAANGSAMGIVGDSPLVKRLIATAQVVARSNST

SEQ ID NO: 59 - protein, 225 aa, Rhizobium etli (strain CFN 42 / ATCC 51251) - P54931
TQRSGIYEISKVLTAPARLEITLANVVNVLSSFLQIRCGAIVVLDAEGQPEIAATGDIPPSSQSAARGVIPKAVIDHIATTGMPLIVKDVSKSELFQAEPQPPWSSGTVPISFIGVPVKADNKILGTISIDRVRNDAAPFPADEDVRFLTMVANLVSRTIRLHRFLNLEGRRPIGEQERPEKPIIAQRGAPGRHPPVKIDGIIGDSPALQQVVETVSVVARTNST

SEQ ID NO: 60 - protein, 225 aa, Bradyrhizobium diazoefficiens (strain JCM 10833 - P05407
SALAGIYEISKILNAPGRLEVTLANVLGLLQSFVQMRHGLVSLFNDDGVPELTVGAGWSEGTDERYRTCVPQKAIHEIVATGRSLMVENVAAETAFSAADREVLGASDSIPVAFIGVPIRVDSTVVGTLTIDRIPEGSSSLLEYDARLLAMVANVIGQTIKLHRLFAGDREQSLVDKDRLEKQTVDRGPPARERKQLQAHGIIGDSPALSALLEKIVVVARSNST

SEQ ID NO: 61 - protein, 218 aa, Rhizobium meliloti (strain 1021) - P03028
KRSAEIYSISKALMAPTRLETTLNNFVNTLSLILRMRRGGLEIPASEGETKITAATRNSGSPSAADYTVPKAAIDQVMTAGRLVVPDVCNSELFKDQIKWRGIGPTAFIAAAVEVDHETGGMLWFECAEESDYDYEEEVHFLSMAANLAGRAIRLHRTISRRERTFAEEQQEQQNSRDEQSQSSARQRLLKNDGIIGESTALMTAVDTAKVMAETNSI

SEQ ID NO: 62 - protein, 215 aa, Herbaspirillum seropedicae - P27713
LELVTIYEISKILGSSLDLSKTLREVLNVLSAHLETKRVLLSLMQDSGELQLVSAIGLSYEEFQSGRYRVGEGITGKIFQTETPIVVRDLAQEPLFLARTSPRQSQDGEVISFVGVPIKAAREMLGVLCVFRDGQSPSRSVDHEVRLLTMVANLIGQTVRLYRSVAAERQQLQEEKRQLSRQLQGKYKLDNVIGISKAMQEVFAQVHQSAPSRST

SEQ ID NO: 63 - protein, 224 aa, Sinorhizobium fredii (strain NBRC 101917) - Q53206
LGCSGLYRISKVLITSASLEIKLANVINTLPALLPMRRGAIVVVGAEGEPETTATFGVEPPSSGARHIAAKAAIDRIVAKGAPLVVPDTCKSELFQDELQSIVSGTGQVTFIGVPMKADQETLGTLWIDRAKDGAATRTQFEEEVRFLSMVANLAARAVRLNGHESRDSRPIGEEGDRKISGGDKELPEPTRQRPTRIDWIVGESPALRQVVESVKVVAQTNSA

SEQ ID NO: 64 - protein, 215 aa, Azospirillum lipoferum - P54929
LELLTIYEVSKILGSSLDLQQTLREVLRALAYQLQMHRGRVYLVGEDNVLRLVAANGLSNEAAAQIEFRDGEGITGRILKTGMPAVVPNLAEEPLFLNRTGGREDLDEQVASLVGVPIKAAGVVVGVLTIDRISDEGPQGHFGSDVRFLTMVANLIGQTVRLPHVAEEPLRDAETFRMQKELRPIAAPINDVVCTSPNMLEVMAQVHRVAPFKST

SEQ ID NO: 65 - protein, 195 aa, Rhizobium leguminosarum - P09828
ARLHILYDISKELISSFPLDNLLKAAMNALVEHLRLRDGGIVIHGSGGEPWINVRAPIGDDVRSRSLTIEQADAINRVIASGEKHFGKNSVVLPVKVNRKAIGALWIDFAQKSGAQDESLLAMIAVLIGLTCQRDRELCSDGGSVAEEQQAGQIPKIKPKPHPTQLDKIDWIVGESPALKRVLATTKIVAATNSA

SEQ ID NO: 66 - protein, 214 aa, Azotobacter vinelandii - P09570
QLQALASIARTLSREQQIDELLEQVLAVLHNDLGLLHGLVTISDPEHGALQIGAIHTDSEAVAQACEGVRYRSGEGVIGNVLKHGNSVVLGRISADPRFLDRLALYDLEMPFIAVPIKNPEGNTIGVLAAQPDCRADEHMPARTRFLEIVANLLAQTVRLVVNIEDGREAADERDELRREVRGKYGFENMVVGHTPTMRRVFDQIRRVAKWNST

SEQ ID NO: 67 - protein, 218 aa, Klebsiella pneumoniae - P03027
QQFTAMQRISVVLSRATEASKTLQEVLSVLHNDAFMQHGMICLYDSQQEILSIEALQQTEDQTLPGSTQIRYRPGEGLVGTVLAQGQSLVLPRVADDQRFLDRLSLYDYDLPFIAVPLMGPHSRPIGVLAAHAMARQEERLPACTRFLETVANLIAQTIRLMILPTSAAQAPQQSPRIERPRACTPSRGFGLENMVGKSPAMRQIMDIIRQVSRWDTT

SEQ ID NO: 68 - protein, 210 aa, Enterobacter agglomerans - P54930
AQFTALYRISVALSQESNTARALAAILEVLHDHAFMQYGMVCLFDKERNALFVESLHGIDGERKKETRHVRYRMGEGVIGAVMSQRQALVLPRISDDQRFLDRLNIYDYSLPLIGVPIPGADNQPAGVLVAQPMALHEDRLAASTRFLEMVANLISQPLRSATPPESLPAQTPVRCSVPRQFGFEQMVGKSQAMRQTMDILRQVSKWDTT

SEQ ID NO: 69 - protein, 35 aa, Rhizobium leguminosarum bv. Trifolii - P24426
MPKSTVRPGAEIVGESAALKEVLEIAQIVARSNSP

SEQ ID NO: 70 - protein, 218 aa, Klebsiella oxytoca - P56266
QQFTAMQRISVVLSRATEASKTLQEVLSVLHNDAFMQHGMICLYDSQQEILSIEALQQTENQTLPGSTQIRYRPGEGLVGTVLAQGQSLVLPRVADDQRFLDRLSLYDYDLPFIAVPLMGPHSRPIGVLAAQPMARQEERLPACTRFLETVANLIAQTIRLMILPTSAAQPPQQSPRVERPRACTSSRGFGLENMVGKSPAMRQIMDIIRQVSRWDTT

SEQ ID NO: 71 - protein, 199 aa, Azospirillum lipoferum
MPSAAQSASSSSLELLTIYEVSKILGSSLDLEQTLREVLRALSYQLQMHRGRVYLQGEDGALRLVAAQGLPKDSPAQEIDYNQGEGISGRILKTGMPAVVPNLAEEPLFNNRSGGRDDLDEQVASLVGVPIKAAGTVIGVLTIDRISDDGPSGHFGSDVRFLTMVANLIGQTVRLHRTVAEERRFMMRETFRVQKELRP

SEQ ID NO: 72 - protein, 195 aa, Paraburkholderia xenovorans
MLNIQATVARQALDSVYEVSKTLVSSLDVAKTFRESLNYLLHTMEWRRAFVVLAEPDGQLRGLCAAGLSRDEQQRLQFRPGEGIVGRAFKSGIGVIVPDVNDEPVFLNRTGGADQMPGACIAMLATPIHADRRTLGVLAVDCVNPGASRLFADDLHLMKIVATLMGQALLLQRSVSAAHDSMQDEVRRMQKAIKP

SEQ ID NO: 73 - protein, 60 aa, Azospirillum lipoferum - CI1666
VLIRGESGTGKELIARAIHNLSPRKDAPFIRVNCAALPEALLESELFGHEKGSFTGAQKD

SEQ ID NO: 74 - protein, 60 aa, Paraburkholderia xenovorans - CI3044
VLLRGESGTGKEVIARAIHNLSPRSGDSFVRVNCAALTESLLESELFGHEKGAFTGAQGQ

SEQ ID NO: 75 - protein, 60 aa, Rhodobacter capsulatus (strain ATCC BAA-309) - D5ARW9
VLLRGESGTGKELFARAVHAQSPRAKGPFIRVNCAALSETLLESELFGHEKGAFTGATAL

SEQ ID NO: 76 - protein, 60 aa, Rhodobacter capsulatus (Rhodopseudomonas capsulata) P0CY94; identical to SEQ ID NO: 75

SEQ ID NO: 77 - protein, 60 aa, Azospirillum brasilense - P30667
VLIRGESGTGKELIARAIHNMSPRKDAPFIRVNCAALPESLLESELFGHEKGAFTGAQKD

SEQ ID NO: 78 - protein, 60 aa, Azorhizobium caulinodans (strain ATCC 43989 ) - P09133
VLLRGESGTGKELFARAIHELSPRKGKPFVKVNCAALPESVLESELFGHEKGAFTGALNM

SEQ ID NO: 79 - protein, 60 aa, Rhizobium etli (strain CFN 42 / ATCC 51251) - P54931
VLLRGESGTGKEFFAQAIHELSPRKNKPFVKLNCAALPEGVLESELFGHEKGAFTGAIAQ

SEQ ID NO: 80 - protein, 60 aa, Bradyrhizobium diazoefficiens (strain JCM 10833 - P05407
VLLRGESGTGKELVAKAIHESSVRAKRPFVKLNCAALPETVLESELFGHEKGAFTGAVSA

SEQ ID NO: 81 - protein, 60 aa, Rhizobium meliloti (strain 1021) - P03028
VLLRGETGTGKECFAKLIHQHSTRQKKPFIKFNCPALSESLLESELFGHEKGAFTGAIAQ

SEQ ID NO: 82 - protein, 60 aa, Herbaspirillum seropedicae - P27713
MLLRGESGTGKEVIARAIHYLSPRKDGPFIKVNCAALSETLLESELFGHEKGAFTGAQGE

SEQ ID NO: 83 - protein, 60 aa, Sinorhizobium fredii (strain NBRC 101917) - Q53206
VLLRGESGTGKEFFAKAIHELSPRRKKPFVKLNCAALSAGVLESELFGHEKGAFTGAISQ

SEQ ID NO: 84 - protein, 60 aa, Azospirillum lipoferum - P54929; identical to SEQ ID NO: 77

SEQ ID NO: 85 - protein, 60 aa, Rhizobium leguminosarum - P09828
VLLRGESGTGKECFARAIHALSIRKSKAFIKLNCAALSETVLESELFGHEKGAFTGALLQ

SEQ ID NO: 86 - protein, 60 aa, Azotobacter vinelandii - P09570
VLVLGESGTGKELIASAIHYKSPRAHRPFVRLNCAALPETLLESELFGHEKGAFTGAVKQ

SEQ ID NO: 87 - protein, 60 aa, Klebsiella pneumoniae - P03027
VLVRGESGTGKELIANAIHHNSPRAAAAFVKFNCAALPDNLLESELFGHEKGAFTGAVRQ

SEQ ID NO: 88 - protein, 60 aa, Enterobacter agglomerans - P54930
VLVRGESGTGKELIANAIHYNSPRAAAPFVKFNCAALPDNLLESELFGHEKGAFTGAIRT

SEQ ID NO: 89 - protein, 60 aa, Rhizobium leguminosarum bv. Trifolii - P24426
VLLRGESGTGKEFFAKLIHDSSSRHEKPFVKLNCAALSAGVLESELFGHEKGAFTGATSQ

SEQ ID NO: 90 - protein, 61 aa, Klebsiella oxytoca - P56266
VLVRGESGTGKELIANAIHHNSPRAAAAFVKFNCAALPDNLLESELFGHEKGAFTGAVRQR

SEQ ID NO: 91 - protein, 603 aa, Azospirillum halopraeferens; identical to SEQ ID NO: 43

(19) United States
(12) Patent Application Publication
(10) Pub. No.: US 2024/0294953 A1
(43) Pub. Date: Sep. 5, 2024

(54) GENETICALLY-ENGINEERED BACTERIAL STRAINS FOR IMPROVED FIXATION OF NITROGEN

(71) Applicant: Pivot Bio, Inc., Berkeley, CA (US)

(72) Inventors: Bilge Ozaydin Eskiyenenturk, Berkeley, CA (US); Min-Hyung Ryu, Berkeley, CA (US); Jenny Johnson, Berkeley, CA (US); Leland Wong, Berkeley, CA (US)

(21) Appl. No.: 18/575,490

(22) PCT Filed: Jun. 30, 2022
(86) PCT No.: PCT/US2022/035873

§ 371 Date: Dec. 29, 2023

Related U.S. Application Data
(60) Provisional application No. 63/218,043, filed on Jul. 2, 2021.

(51) Int. Cl. C12P 13/00; C12N 1/20; C12N 9/02; C12N 15/10; C12N 15/74; C12R 1/01

(52) U.S. Cl. CPC C12P 13/00; C12N 1/205; C12N 9/0095; C12N 15/1082; C12N 15/74; C12Y 118/06001; C12N 2800/101; C12R 2001/01

(57) ABSTRACT

Methods and systems are provided for generating and utilizing a genetically engineered bacterium comprising a modification in a nifA gene or homolog thereof that can result in a bacterium with modified regulation of nitrogen fixation or assimilation activity. Genetically engineered bacteria with modified nitrogen fixation or assimilation activity are also provided. The genetically engineered bacterium can fix nitrogen in the presence of nitrogen (e.g., ammonium), and/or oxygen.

### CROSS-REFERENCE TO RELATED APPLICATIONS

This application claims benefit of priority from U.S. Provisional Application Ser. No. 63/218,043, filed Jul. 2, 2021. The disclosure of the prior application is considered part of (and is incorporated by reference in) the disclosure of this application.

### TECHNICAL FIELD

The present disclosure is related to genetically-engineered bacterial strains, and compositions thereof. Such bacterial strains, and compositions thereof, are useful for providing nitrogen to plants.

### STATEMENT REGARDING SEQUENCE LISTING

This application contains a Sequence Listing that has been submitted electronically as an ASCII text file named 486240025WO1_ST25.txt. The ASCII text file, created on Jun. 30, 2022, is 175 KB bytes in size. The material in the ASCII text file is hereby incorporated by reference in its entirety.

### BACKGROUND

Approaches to agriculture and food production that are economically, environmentally, and socially sustainable will help to meet the needs of a growing global population. By 2050, the United Nations' Food and Agriculture Organization projects that total food production must increase by 70% to meet the needs of the growing population, a challenge that can be exacerbated by numerous factors, including diminishing freshwater resources, increasing competition for arable land, rising energy prices, increasing input costs, and the likely need for crops to adapt to the pressures of a drier, hotter, and more extreme global climate.

One area of interest is in the improvement of nitrogen fixation. Nitrogen gas (N₂) is a major component of the atmosphere of Earth. In addition, elemental nitrogen (N) is an important component of many chemical compounds which make up living organisms. However, many organisms cannot use N₂ directly to synthesize the chemicals used in physiological processes, such as growth and reproduction. N₂ must be combined with hydrogen to be utilized. This process of combining of hydrogen with N₂ is referred to as nitrogen fixation. Nitrogen fixation, whether accomplished chemically or biologically, requires an investment of large amounts of energy. In biological systems, the enzyme known as nitrogenase catalyzes the reaction which results in nitrogen fixation. An important goal of nitrogen fixation research is the extension of this phenotype to non-leguminous plants, particularly to important agronomic grasses such as wheat, rice, and maize. Despite enormous progress in understanding the development of the nitrogen-fixing symbiosis between rhizobia and legumes, the path to use that knowledge to induce nitrogen-fixing nodules on non-leguminous crops is still not clear. Meanwhile, the challenge of providing sufficient supplemental sources of nitrogen, such as in fertilizer, will continue to increase with the growing need for increased food production.

### SUMMARY

This document is based, at least in part, on identification of one or more targeted genomic modifications to the nifA gene that can be used to produce genetically engineered bacteria that can fix nitrogen under both nitrogen limiting and non-nitrogen limiting conditions (e.g., in the presence of ammonia) as well as in the presence of oxygen (e.g., at least 0.5% oxygen). Such genetically engineered bacteria can be used in methods to increase the amount of atmospheric derived nitrogen in plants (e.g., non-leguminous plants such as corn, wheat, sorghum, and rice).

Provided herein are engineered microbes includes one or more genetic modifications in a gene encoding a NifA polypeptide, wherein the engineered microbe fixes nitrogen in the presence exogenous nitrogen and oxygen.

Also provided herein are compositions including a plurality of any of the engineered microbes described herein, and a plant seed.

Also provided herein are methods of increasing an amount of ammonium production of a microbe, the method comprising engineering the microbe to include one or more genetic modifications in a gene encoding a NifA polypeptide, wherein the engineered microbe fixes nitrogen in the presence of nitrogen and oxygen.

Also provided herein are plants or plant parts including any of the engineered microbes described herein.

Also provided herein are methods of increasing an amount of atmospheric derived nitrogen in a plant in a field, the method comprising contacting a soil, a plant, or a plant seed with a plurality of the engineered microbes described herein. In some embodiments, the plurality of engineered microbes are coated onto the plant seed. In some embodiments, the plurality of engineered microbes are applied into furrows in which seeds of the plant are planted.

In some embodiments, the NifA polypeptide encoded by the gene with one or more genetic modifications exhibits increased transcriptional activation of nitrogen fixation genes in the presence of nitrogen and oxygen relative to that of a wild-type NifA polypeptide in the presence of nitrogen and oxygen. In some embodiments, the exogenous nitrogen includes ammonium, nitrate, urea, or glutamine. In some embodiments, the NifA polypeptide encoded by the gene with one or more genetic modifications overcomes ammonium inhibition in the presence of nitrogen.

In some embodiments, the NifA polypeptide encoded by the gene with one or more genetic modifications includes a substitution at one or more amino acid positions corresponding to amino acids 23 or 164 of SEQ ID NO: 15 or at homologous amino acid position(s) in a homolog thereof. In some embodiments, the amino acid corresponding to position 23 of SEQ ID NO: 15 or at a homologous amino acid position in a homolog thereof is substituted with a non-positively charged amino acid. In some embodiments, the amino acid corresponding to position 23 of SEQ ID NO: 15 or at a homologous amino acid position in a homolog thereof is the amino acid D or E. In some embodiments, the amino acid corresponding to position 164 of SEQ ID NO: 15 or at a homologous amino acid position in a homolog thereof is substituted with an amino acid lacking sulfur. In some embodiments, the amino acid corresponding to position 164 of SEQ ID NO: 15 or at a homologous amino acid position in a homolog thereof is the amino acid I, L, or T.

In some embodiments, the NifA polypeptide encoded by the gene with one or more genetic modifications comprises a substitution at one or more amino acid positions corresponding to amino acids 7, 21, 34, 42, 93, 108, 116, 122, 159, 166, 178, 185, 186, or 196 of SEQ ID NO: 14 or at one or more homologous positions in a homolog thereof.

In some embodiments, the substitution is at amino acid positions corresponding to the following amino acids of SEQ ID NO: 14 or homologous amino acid positions in a homolog thereof: 108, 159, 166, and 185; 42, 122, and 166; 42 and 178; 186 and 196; or 7, 34, 93, 116, and 178.

In some embodiments, the amino acid corresponding to position 7 of SEQ ID NO: 14 or a homologous amino acid position in a homolog thereof is substituted with the amino acid D. In some embodiments, the amino acid corresponding to position 34 of SEQ ID NO: 14 or a homologous amino acid position in a homolog thereof is substituted with the amino acid E. In some embodiments, the amino acid corresponding to position 42 of SEQ ID NO: 14 or a homologous amino acid position in a homolog thereof is substituted with the amino acid D or S. In some embodiments, the amino acid corresponding to position 93 of SEQ ID NO: 14 or a homologous amino acid position in a homolog thereof is substituted with the amino acid E or V. In some embodiments, the amino acid corresponding to position 108 of SEQ ID NO: 14 or a homologous amino acid position in a homolog thereof is substituted with the amino acid E. In some embodiments, the amino acid corresponding to position 116 of SEQ ID NO: 14 or a homologous amino acid position in a homolog thereof is substituted with the amino acid H. In some embodiments, the amino acid corresponding to position 122 of SEQ ID NO: 14 or a homologous amino acid position in a homolog thereof is substituted with the amino acid E. In some embodiments, the amino acid corresponding to position 159 of SEQ ID NO: 14 or a homologous amino acid position in a homolog thereof is substituted with the amino acid T. In some embodiments, the amino acid corresponding to position 166 of SEQ ID NO: 14 or a homologous amino acid position in a homolog thereof is substituted with the amino acid A. In some embodiments, the amino acid corresponding to position 178 of SEQ ID NO: 14 or a homologous amino acid position in a homolog thereof is substituted with the amino acid A or M. In some embodiments, the amino acid corresponding to position 185 of SEQ ID NO: 14 or a homologous amino acid position in a homolog thereof is substituted with the amino acid T. In some embodiments, the amino acid corresponding to position 186 of SEQ ID NO: 14 or a homologous amino acid position in a homolog thereof is substituted with the amino acid R. In some embodiments, the amino acid corresponding to position 196 of SEQ ID NO: 14 or a homologous amino acid position in a homolog thereof is substituted with the amino acid V. In some embodiments, the amino acid corresponding to position 121 of SEQ ID NO: 14 or at a homologous amino acid position in a homolog thereof is substituted with the amino acid A, and wherein the amino acid corresponding to position 166 of SEQ ID NO: 14 or at a homologous amino acid position in a homolog thereof is substituted with the amino acid A. In some embodiments, the amino acid corresponding to position 21 of SEQ ID NO: 14 or at a homologous amino acid position in a homolog thereof is substituted with the amino acid E.

In some embodiments, the substitution is at amino acids corresponding to the following amino acids of SEQ ID NO: 14 or at homologous amino acid positions in a homolog thereof: D108E, D159T, T166A, and M185T; N42D, D122A, and T166A; N42S and V178A; Q186R and 1196V; or G7D, R34E, M93V, P116H, and V178M.

In some embodiments, the NifA polypeptide encoded by the gene with one or more genetic modifications comprises a substitution at amino acids corresponding to S28P, M96T, and M164L of SEQ ID NO: 14 or at homologous amino acid positions in a homolog thereof. In some embodiments, the NifA polypeptide encoded by the gene with one or more genetic modifications comprises a substitution at amino acids corresponding to Q186R and 1196V of SEQ ID NO: 14 or at homologous amino acid positions in a homolog thereof. In some embodiments, the NifA polypeptide encoded by the gene with one or more genetic modifications comprises a substitution at an amino acid corresponding to N42E of SEQ ID NO: 14 or at a homologous amino acid position in a homolog thereof. In some embodiments, the NifA polypeptide encoded by the gene with one or more genetic modifications comprises a substitution at one or more amino acid positions corresponding to amino acids 16, 23, 26, 28, 37, 65, 72, 93, 96, 123, 158, 164, 171, 183, or 209 of SEQ ID NO: 15 or at homologous amino acid position(s) in a homolog thereof.

In some embodiments, the substitution is at amino acid position(s) corresponding to the following amino acids of SEQ ID NO: 15 or at homologous amino acid position(s) in a homolog thereof: 37, 65, 93, 164, and 209; 16, 23, 72, 158, 171, and 183; 28, 96, and 164; 23, 148, and 164; 123 and 164; 26; or 23.

In some embodiments, the amino acid corresponding to position 16 of SEQ ID NO: 15 or a homologous amino acid position in a homolog thereof is substituted with the amino acid P. In some embodiments, the amino acid corresponding to position 24 of SEQ ID NO: 15 or a homologous amino acid position in a homolog thereof is substituted with the amino acid E. In some embodiments, the amino acid corresponding to position 26 of SEQ ID NO: 15 or a homologous amino acid position in a homolog thereof is substituted with the amino acid E. In some embodiments, the amino acid corresponding to position 28 of SEQ ID NO: 15 or a homologous amino acid position in a homolog thereof is substituted with the amino acid P. In some embodiments, the amino acid corresponding to position 37 of SEQ ID NO: 15 or a homologous amino acid position in a homolog thereof is substituted with the amino acid G. In some embodiments, the amino acid corresponding to position 65 of SEQ ID NO: 15 or a homologous amino acid position in a homolog thereof is substituted with the amino acid A. In some embodiments, the amino acid corresponding to position 72 of SEQ ID NO: 15 or a homologous amino acid position in a homolog thereof is substituted with the amino acid E. In some embodiments, the amino acid corresponding to position 93 of SEQ ID NO: 15 or a homologous amino acid position in a homolog thereof is substituted with the amino acid E or V. In some embodiments, the amino acid corresponding to position 96 of SEQ ID NO: 15 or a homologous amino acid position in a homolog thereof is substituted with the amino acid T. In some embodiments, the amino acid corresponding to position 124 of SEQ ID NO: 15 or a homologous amino acid position in a homolog thereof is substituted with the amino acid E. In some embodiments, the amino acid corresponding to position 158 of SEQ ID NO: 15 or a homologous amino acid position in a homolog thereof is substituted with the amino acid N or T. In some embodiments, the amino acid corresponding to position 164 of SEQ ID NO: 15 or a homologous amino acid position in a homolog thereof is substituted with the amino acid I, L, or T. In some embodiments, the amino acid corresponding to position 171 of SEQ ID NO: 15 or a homologous amino acid position in a homolog thereof is substituted with the amino acid K. In some embodiments, the amino acid corresponding to position 183 of SEQ ID NO: 15 or a homologous amino acid position in a homolog thereof is substituted with the amino acid Q. In some embodiments, the amino acid corresponding to position 209 of SEQ ID NO: 15 or a homologous amino acid position in a homolog thereof is substituted with the amino acid R. In some embodiments, the amino acid corresponding to position 23 of SEQ ID NO: 15 or a homologous amino acid position in a homolog thereof is substituted with the amino acid E.

In some embodiments, the substitution is at amino acid(s) corresponding to the following amino acid(s) of SEQ ID NO: 15 or at homologous amino acid position(s) in a homolog thereof: E37G, V65A, K93E, M164T, and C209R; L16P, K23E, K72E, D158N, Q171K, and R183Q; S28P, M96T, and M164L; K23E, D148G, and M164I; K123E and M164T; G26E; or K23E.

In some embodiments, the NifA polypeptide further comprises a deletion of amino acids corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200 or at homologous amino acid positions in a homolog thereof, or a deletion of the GAF domain of the NifA polypeptide.

In some embodiments, the NifA polypeptide encoded by the gene with one or more genetic modifications comprises a substitution at an amino acid corresponding to N42E of the SEQ ID NO: 14 or at a homologous amino acid position in a homolog thereof and a deletion of amino acids corresponding to amino acids 188-198 of the SEQ ID NO: 14 or at homologous amino acid positions in a homolog thereof.

In some embodiments, the microbe further comprises one or more genetic modifications in a nitrogen fixation and/or a nitrogen assimilation pathway. In some embodiments, the one or more genetic modifications in the nitrogen fixation and/or the nitrogen assimilation pathway result in altered activity of NifH, GlnK, GlnD, GlnE, or a combination thereof. In some embodiments, the one or more genetic modifications in the nitrogen fixation and/or the nitrogen assimilation pathway are a deleted glnK gene, a glnD gene encoding a GlnD polypeptide lacking a UTase domain, a glnE gene encoding a GlnE polypeptide lacking an AR domain, or a combination thereof. In some embodiments, the one or more genetic modifications comprises an insertion of a regulatory element.

In some embodiments, the one or more genetic modifications comprise a deletion of amino acids in the NifA polypeptide corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200; or a deletion of the GAF domain of the NifA polypeptide; and an insertion of a regulatory element operably linked to the nufA gene.

In some embodiments, the regulatory element is a promoter. In some embodiments, the promoter is an acnB promoter, a cps promoter, a gapA1 promoter, a glt promoter, a groS promoter, an infC promoter, an ompA promoter, an oprF promoter, a pflB promoter, a pgk2 promoter, a ppsA promoter, a rpl promoter, a rpmB promoter, a rpoBC promoter, a rps promoter, or a tufA-2 promoter. In some embodiments, the cps promoter comprises a cspA3 promoter, a cspA5 promoter, a cpsD promoter, a cpsD-1 promoter, a cpsD2 promoter, or a cspJ promoter. In some embodiments, the gltA promoter comprises a gltA1 promoter or a gltA2 promoter. In some embodiments, the rps promoter comprises a rpsL promoter or a rpsF promoter. In some embodiments, the rpl promoter comprises a rplL promoter or a rplM promoter. In some embodiments, the regulatory element is a constitutive promoter. In some embodiments, the regulatory element is an inducible promoter. In some embodiments, the regulatory element is a synthetic promoter. In some embodiments, the synthetic promoter is encoded by SEQ ID NO: 3.

In some embodiments, the regulatory element is derived from a microbe of the same species as the engineered microbe. In some embodiments, the regulatory element is derived from a microbe of the same genus as the engineered microbe. In some embodiments, the regulatory element is derived from a microbe of a different species than the engineered microbe. In some embodiments, the regulatory element is derived from a microbe of a different genus than the engineered microbe.

In some embodiments, the engineered microbe is an engineered bacterium. In some embodiments, the engineered microbe is a Proteobacterium. In some embodiments, the engineered microbe is an alpha-Proteobacterium or a beta-Proteobacterium. In some embodiments, the engineered bacterium is selected from the group consisting of: Paraburkholderia spp., Azospirillum spp., and Herbaspirillum spp. In some embodiments, the engineered microbe is a biocontrol microbe.

In some embodiments, the microbe (e.g., an engineered microbe) is a strain of Azospirillum lipoferum deposited in ATCC under Accession No. PTA-127320. In some embodiments, the microbe (e.g., an engineered microbe) is a strain of Azospirillum lipoferum deposited in ATCC under Accession No. PTA-127323. In some embodiments, the the microbe (e.g. an engineered microbe) is a strain of Paraburkholderia tropica deposited in ATCC under Accession No. PTA-127322 or PTA-127321. In some embodiments, the microbe (e.g., an engineered microbe) is a strain of Paraburkholderia xenovorans deposited in ATCC under Accession No. PTA-127325 or PTA-127319.

In some embodiments, the plant seed is a non-leguminous plant seed. In some embodiments, the plant seed is a cereal plant seed. In some embodiments, the plant seed is a seed of a plant selected from the group consisting of: barley, canola, corn, peanut, rice, sorghum, soybean, turfgrass, and wheat.

In some embodiments, the method also includes contacting a soil, a plant, or a plant seed with a plurality of the engineered microbes described herein. In some embodiments, the plurality of engineered microbes are coated onto the plant seed. In some embodiments, the plurality of engineered microbes are applied into furrows in which seeds of the plant are planted.

In some embodiments, the plant seed is a non-leguminous plant seed. In some embodiments, the plant seed is a cereal plant seed. In some embodiments, the plant seed is a seed of a plant selected from the group consisting of: barley, canola, corn, peanut, rice, sorghum, soybean, turfgrass, and wheat.

In some embodiments, the microbe is strain Azospirillum lipoferum deposited in ATCC under Accession No. PTA-127320.

### Definitions

The terms “polynucleotide,” “nucleotide sequence,” “nucleic acid” and “oligonucleotide” are used interchangeably. They refer to a polymeric form of nucleotides of any length, either deoxyribonucleotides or ribonucleotides, or analogs thereof. Polynucleotides can have any three dimensional structure, and can perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide can comprise one or more modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, can be calculated as the number of exact matches between two aligned sequences divided by the length of the shorter sequences and multiplied by 100. In some embodiments, the percent identity of a test sequence and a reference sequence, whether nucleic acid or amino acid sequences, can be calculated as the number of exact matches between two aligned sequences divided by the length of the reference sequence and multiplied by 100. Percent identity can also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program can be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values there between. Typically, the percent identities between a disclosed sequence and a claimed sequence are at least 80%, at least 85%, at least 90%, at least 95%, at least 98% or at least 99%.

Sequences can be aligned using an algorithm including but not limited to the Needleman-Wunsch algorithm (see e.g. the EMBOSS Needle aligner available at ebi.ac.uk/Tools/psa/emboss_needle/nucleotide.html on the World Wide Web, optionally with default settings), the BLAST algorithm (see e.g. the BLAST alignment tool available at blast.ncbi.nlm.nih.gov/Blast.cgi on the World Wide Web, optionally with default settings), or the Smith-Waterman algorithm (see e.g. the EMBOSS Water aligner available at ebi.ac.uk/Tools/psa/emboss_water/nucleotide.html on the World Wide Web, optionally with default settings). Optimal alignment can be assessed using any suitable parameters of a chosen algorithm, including default parameters.

As used herein, “expression” refers to the process by which a polynucleotide is transcribed from a DNA template (such as into an mRNA or other RNA transcript) and/or the process by which a transcribed mRNA is subsequently translated into a peptide, polypeptide, or protein. Transcripts and encoded polypeptides can be collectively referred to as “gene product.” If the polynucleotide is derived from genomic DNA, expression can include splicing of the mRNA in a eukaryotic cell.

The terms “polypeptide”, “peptide” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

As used herein the term “amino acid” includes natural (e.g., alpha-amino acids) and unnatural or synthetic amino acids, including both the D or L optical isomers, amino acid analogs, and peptidomimetics. Amino acids can be positively charged or negatively charged. Amino acids can be not positively charged, or not negatively charged. Amino acids can contain or lack sulfur. Non-limiting examples of unnatural amino acids include beta-amino acids, homo-amino acids, proline and pyruvic acid derivatives, 3-substituted alanine derivatives, glycine derivatives, ring substituted phenylalanine and tyrosine derivatives, linear core amino acids, and N-methyl amino acids. An amino acid analog can be an amino acid resulting from a reaction at an amino group, carboxy group, side-chain functional group, or from the replacement of any hydrogen by a heteroatom.

As used herein, the term “about” is used synonymously with the term “approximately.” Illustratively, the use of the term “about” with regard to an amount indicates that values slightly outside the cited values, e.g., plus or minus 0.1% to 10%.

The term “biologically pure culture” or “substantially pure culture” refers to a culture of a bacterial species described herein containing no other bacterial species in quantities sufficient to interfere with the replication of the culture or be detected by normal bacteriological techniques.

As used herein the term “plant” can include plant parts such as tissues, leaves, roots, root hairs, rhizomes, stems, seeds, ovules, pollen, flowers, fruit, etc.

As used herein, “in planta” may refer to in the plant, on the plant, or intimately associated with the plant, depending upon context of usage (e.g. endophytic, epiphytic, or rhizospheric associations). The plant may comprise plant parts such as tissue, leaves, roots, root hairs, rhizomes, stems, seed, ovules, pollen, flowers, fruit, etc.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, reducing NO₂ emission due to reduced nitrogen fertilizer usage and similar improvements of the growth and development of plants.

In some embodiments, “applying to the plant a plurality of genetically engineered bacteria,” or “applying to the plant a plurality of engineered microbes” such as non-intergeneric bacteria includes any means by which the plant (including plant parts such as a seed, root, stem, tissue, etc.) is made to come into contact (i.e., exposed) with said bacteria at any stage of the plant's life cycle. Consequently, “applying to the plant a plurality of genetically engineered bacteria,” includes any of the following means of exposing the plant (including plant parts such as a seed, root, stem, tissue, etc.) to said bacteria: spraying onto plant, dripping onto plant, applying as a seed coat, applying to a field that will then be planted with seed, applying to a furrow that will then be planted with seed, applying to a field already planted with seed, applying to a field with adult plants, etc.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria (e.g., a non-engineered bacteria of the same species). All increases or decreases in plants are measured relative to control plants.

As used herein, a “control sequence” refers to an operator, promoter, silencer, or terminator.

In some embodiments, native or endogenous control sequences of genes of the present disclosure are replaced with one or more intrageneric control sequences.

As used herein, a “constitutive promoter” is a promoter that is active under most conditions and/or during most developmental stages. There can be several advantages to using constitutive promoters in expression vectors used in biotechnology. Such advantages can include a high level of production of proteins used to select transgenic cells or organisms; a high level of expression of reporter proteins or scorable markers that can allow easy detection and quantification; a high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter which is active under certain conditions, in certain types of cells, and/or during certain development stages. For example, tissue-specific promoters, tissue-preferred promoters, cell type-specific promoters, cell type-preferred promoters, inducible promoters, and promoters under developmental control are non-constitutive promoters. Examples of promoters under developmental control include promoters that preferentially initiate transcription in certain tissues.

As used herein, an “inducible” promoter or a “repressible” promoter is a promoter that is under the control of chemical or environmental factors. Examples of environmental conditions that can affect transcription by inducible promoters include anaerobic conditions, certain chemicals, the presence of light, acidic or basic conditions, etc.

As used herein, a “tissue-specific” promoter is a promoter that initiates transcription only in certain tissues. Unlike constitutive expression of genes, tissue-specific expression is the result of several interacting levels of gene regulation. It can be advantageous to use promoters from homologous or closely related species to achieve efficient and reliable expression of transgenes in particular tissues. This is a reason for the large amount of tissue-specific promoters isolated from particular tissues found in both scientific and patent literature.

As used herein, the term “operably linked” refers to the association of nucleic acid sequences on a single nucleic acid fragment such that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In some embodiments, complementary RNA regions of the disclosure are operably linked either directly or indirectly, for example, 5′ to the target mRNA, 3′ to the target mRNA, or within the target mRNA. In some embodiments, a first complementary region is 5′ and its complement is 3′ to the target mRNA.

As used herein, “introduced” refers to the introduction by means of modern biotechnology, and not a naturally occurring introduction.

As used herein, “introduced genetic material” means genetic material that is added to, and remains as a component of, the genome of the recipient.

As used herein the terms “microorganism” or “microbe” should be taken broadly. These terms, used interchangeably, include but are not limited to, the two prokaryotic domains, Bacteria and Archaea. The term may also encompass eukaryotic fungi.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism

In some embodiments, microbes disclosed herein are “non-intergeneric,” which means that the microbes are not intergeneric.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism.”

As used herein, in the context of non-intergeneric microorganisms, the term “remodeled” is used synonymously with the term “engineered”. Consequently, a “non-intergeneric remodeled microorganism” has a synonymous meaning to “non-intergeneric engineered microorganism,” and will be utilized interchangeably. Further, the disclosure may refer to an “engineered strain” or “engineered derivative” or “engineered non-intergeneric microbe,” these terms are used synonymously with “remodeled strain” or “remodeled derivative” or “remodeled non-intergeneric microbe.” An engineered microorganism contains in its genome at least one genetic modification.

In some embodiments, the bacteria of the present disclosure have been modified such that they are not naturally occurring bacteria.

As used herein, when the disclosure discusses a particular microbial deposit by accession number, it is understood that the disclosure also contemplates a microbial strain having all of the identifying characteristics of said deposited microbe and/or a mutant thereof.

The term “microbial consortia” or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest.

The term “microbial community” means a group of microbes comprising two or more species or strains. Unlike microbial consortia, a microbial community does not have to be carrying out a common function, or does not have to be participating in, or leading to, or correlating with, a recognizable parameter, such as a phenotypic trait of interest.

As used herein, “isolate,” “isolated,” “isolated microbe,” and like terms, are intended to mean that the one or more microorganisms has been separated from at least one of the materials with which it is associated in a particular environment (for example soil, water, plant tissue, etc.). Thus, an “isolated microbe” does not exist in its naturally-occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain).

In some embodiments, the isolated microbe may be in association with an acceptable carrier, which may be an agriculturally acceptable carrier.

In some embodiments, the isolated microbes exist as “isolated and biologically pure cultures.” It will be appreciated by one of skill in the art that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.”

In some embodiments, wherein a plurality of genetically engineered microbes comprising at least one modification in a gene regulating nitrogen fixation or assimilation are provided, at least about 25% of the plurality comprises the at least one modification in a gene regulating nitrogen fixation or assimilation. In some embodiments, at least about 50% of the plurality of genetically engineered microbes comprises the at least one modification in a gene regulating nitrogen fixation or assimilation. For example, at least about 55%, about 60%, about 65%, about 70%, about 75%, about 80%, about 85%, about 90%, about 95%, about 98%, or about 99% of the plurality of genetically engineered microbes comprises the at least one modification in a gene regulating nitrogen fixation or assimilation. In some embodiments, every member of the plurality of genetically engineered microbes comprises the at least one modification.

In some embodiments, the disclosure provides for certain quantitative measures of the concentration, or purity limitations that must be found within an isolated and biologically pure microbial culture. The presence of these purity values, in certain embodiments, is a further attribute that distinguishes the presently disclosed microbes from those microbes existing in a natural state.

As used herein, “individual isolates” should be taken to mean a composition, or culture, comprising a predominance of a single genera, species, or strain, of microorganism, following separation from one or more other microorganisms.

Microbes of the present disclosure can include spores and/or vegetative cells. In some embodiments, microbes of the present disclosure include microbes in a viable but nonculturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be non-conducive to the survival or growth of vegetative cells.

As used herein, a “microbial composition” refers to a composition comprising one or more microbes of the present disclosure. In some embodiments, a microbial composition is administered to plants (including various plant parts) and/or in agricultural fields.

As used herein, “carrier,” “acceptable carrier,” or “agriculturally acceptable carrier” refers to a diluent, adjuvant, excipient, or vehicle with which the microbe can be administered, which does not detrimentally effect the microbe or the plant.

In some embodiments, the microbes (e.g., bacteria) of the present disclosure are present in the plant in an amount of at least 10³ cfu, 10⁴ cfu, 10⁵ cfu, 10⁶ cfu, 10⁷ cfu, 10⁸ cfu, 10⁹ cfu, 10¹⁰ cfu, 10¹¹ cfu, or 10¹² cfu per gram of fresh or dry weight of the plant. In some embodiments, the microbes (e.g., bacteria) of the present disclosure are present in the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu per gram of fresh or dry weight of the plant. In some embodiments, the microbes (e.g., bacteria) of the present disclosure are present in the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu per gram of fresh or dry weight of the plant.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, and other nitrogen species that include an ammonium ion, etc.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “non-nitrogen limiting conditions” refers to non-atmospheric nitrogen available in the soil, field, or culture media at concentrations greater than about 4 mM nitrogen, as disclosed by Kant et al. (2010. J. Exp. Biol. 62(4):1499-1509), which is incorporated herein by reference.

In general, the term “genetic modification” refers to any change introduced into a polynucleotide sequence relative to a reference polynucleotide, such as a reference genome or portion thereof, or reference gene or portion thereof. A genetic modification may be referred to as a “mutation”, and a sequence or organism comprising a genetic modification may be referred to as a “genetic variant”, “mutant”, or “engineered”.

Genetic modifications introduced into microbes can be classified as transgenic, cisgenic, intragenomic, intrageneric, intergeneric, synthetic, evolved, rearranged, or SNPs.

Genetic modification may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion. Nitrogen uptake, glutamine biosynthesis, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C are schematics illustrating a high-throughput screening system for identification of NifA mutants tolerant to ammonium repression. FIG. 1A illustrates a reporter that can be used to assess activation of the nif cluster. The reporter encodes gfp under the nifH promoter and nufA. The plasmid replication origin pRO1600 was used for CI8, and the pBBR1 origin was used for CI1666 to build mutant libraries of NifA. FIG. 1B shows that the GAF domain and the Q-linker were mutagenized by error-prone PCR. FIG. 1C is a schematic illustrating a method for ammonium-insensitive NifA screening. The nifA mutant library is introduced into, for example, E. coli by transformation. The pooled library (>10⁵ recombinants) is transferred into the target species (e.g., Paraburkholderia and Azospirillum) by conjugation. NifA variants that allow the nif cluster to be expressed in nitrogen-replete conditions are identified on the minimal agar media supplemented with 10 mM ammonium chloride. Ammonium derepression by the NifA variants is confirmed by flow cytometry and compared to the NifA wild-type.

FIG. 2 is a graph plotting nifH promoter activity, showing the effect of various NifA variants on nifH promoter activity in strain CI8. The nifH promoter activated by the NifA variants in the presence and absence of ammonium was compared to the nifH promoter activated by wild-type NifA.

FIG. 3 is a graph plotting nifH promoter activity, showing the effect of oxygen on nifH promoter activity in CI8. Expression from the nifH promoter activated by a series of various NifA variants was analyzed in the presence of atmospheric oxygen and ammonium.

FIG. 4 is a graph plotting nifH promoter activity, showing the effect of various Q-linker deletions in NifA on nifH promoter activity in strain CI8. nifH promoter activation by the indicated NifA variants in the presence and absence of ammonium was analyzed by flow cytometry.

FIG. 5 is a graph plotting nifH promoter activity, showing the effect of the combination of the Q-linker deletions and SNPs in the GAF domain of NifA on nifH promoter activity in strain CI8. nifH promoter activation by the indicated NifA variants in the presence and absence of ammonium was analyzed by flow cytometry.

FIG. 6 is a graph plotting nifH promoter activation by various NifA variants in strain CI1666. nifH promoter activation by the indicated NifA variants with the indicated amounts of ammonium and oxygen was analyzed by flow cytometry.

FIGS. 7A-7C are multiple sequence alignments of the N-terminal region of NifA. Conserved residues from the ammonium tolerant NifA mutants are marked with dots. FIG. 7A shows a multiple sequence alignment of the N-terminal region of NifA of strain CI8 across Paraburkholderia species (SEQ ID NOs: 14, and 37-41). FIG. 7B shows a multiple sequence alignment of the N-terminal region of NifA of strain CI1666 across Azospirillum (SEQ ID NOs: 15, and 42-53). FIG. 7C shows a multiple sequence alignment of the N-terminal region of NifA between strains CI8 and CI1666 (SEQ ID NOs: 14-15).

FIG. 8 is a graph plotting ethylene levels, showing the effect of different promoters on NifA activity. Various promoters were inserted upstream of the native nifA coding sequence with a partial deletion of the native upstream sequence. The nifA sequence was either unmodified or had a deletion of the GAF domain. Activity was measured using the ARA assay with ethylene as the output.

FIGS. 9A-9B are plots that show the effects of deleting native upstream sequence during nufA promoter replacement on acetylene reduction (FIG. 9A) and excretion of ammonium (FIG. 9B). The rpsL promoter was inserted upstream of the native nufA in a manner that deleted either 179 bp (v1), 100 bp (v2), or none of the native sequence (v3).

FIG. 10 is a plot that shows the effect of NifA truncations on ammonium excretion. Ammonium excretion in four strains with various deletions of the native nifA sequence; the GAF deletion spans Q2-L202.

FIGS. 11A-11C are plots that show the effect of various NifA modifications on nitrogen fixation (FIG. 11A) and ammonium excretion (FIGS. 11B-11C). All nifA variants showed higher derepressed nitrogenase activity than the wild type nifA.

FIG. 12 is a plot that shows the effect on ammonium excretion as a result of modifications to glnD, glnE, or glnK alone or in conjunction with ΔP(nifA)_v2::P(rpsL)-nifA_ΔGAF.

FIG. 13 is a table that depicts and describes various deletion mutations of nifA that were tested in Herbaspirillum.

FIG. 14 is a plot showing the nitrogenase activity of mutants with the nested N-terminal deletions of H. seropedicae (strain 3000) as measured by an acetylene reduction assay. 3000-5165 and 3000-5121 strains carrying the nifA_ΔA2-G167 and nifA_ΔA2-N202 truncations respectively showed highest activity and derepression of nitrogenase under rich nitrogen conditions.

FIG. 15 is a plot showing the nitrogenase activity of mutants with the GAF domain deletions of H. frisingense (strain 1663) as measured by an acetylene reduction assay. Deletions that showed highest derepression in H. seropedicae (nifA_ΔA2-G167 and nifA_ΔA2-N202 truncations) were tested under various native promoters in H. frisingense.

FIGS. 16A-16B show active promoter characteristics in H. seropedicae using a GFP reporter gene system. FIG. 16A is an image of two exemplary reporter gene system outputs (PcspD-GFP and PoprF-GFP). FIG. 16B is a table that lists the promoters surveyed and their respective strength as determined by GFP levels.

FIGS. 17A-17B are plots showing the nitrogenase activity (FIG. 17A) and ammonium production (FIG. 17B) in H. seropedicae strains expressing nifA variants under constitutive native promoters.

FIG. 18 is a list of A. lipoferum promoters surveyed and their corresponding strength as determined by GFP expression levels.

FIG. 19 is a list and description of various truncations of nifA in Azospirillum.

FIGS. 20A-20D show relatedness of NifA proteins in a variety of bacteria. FIG. 20A is a multiple sequence alignment of NifA proteins (SEQ ID NOs: 54-71). FIG. 20B is a phylogenetic tree for the alignment in FIG. 20A. K23 was conserved across alpha- and beta-proteobacteria, while M164 was conserved only across alpha-proteobacteria. FIG. 20C is a table listing mutations introduced into the CI1666 and CI3044 strains. FIG. 20D is an alignment of the GAF domains of NifA proteins of Azospirillum lipoferum (CI1666, top; SEQ ID NO: 72) and Paraburkholderia xenovorans (CI3044, bottom SEQ ID NO: 73). Starred lysine and methionine residues were chosen for mutational analysis.

FIGS. 21A-21B show GFP expression levels in various strains. FIG. 21A is a graph of GFP expression levels measured by flow cytometry. Strains listed on the x-axis carry a plasmid that encoded a nifH-promoter driven GFP gene (PnifH_GFP). PnifH was active under no nitrogen conditions in all edited strains except when the nufA gene was deleted or not heavily expressed. Under 10 mM NH₄Cl, mutations of the K23 residue to acidic residues (K23D or K23E) showed strong PnifH activity under rich nitrogen conditions. The K132D modification was not sufficient to relive self repression of NifA when introduced individually, but it greatly enhanced NifA activity when combined with K23D (1666-7523). Although methionine modifications did not help with NifA activity under rich nitrogen conditions, they did greatly enhance activity of NifA under no nitrogen conditions, suggesting that methionine modifications may have some self repression activity that is regulated in a nitrogen-independent manner. FIG. 21B is a table listing the strains used in FIG. 21A.

FIGS. 22A-22B show nitrogen levels in various engineered strains. FIG. 22A is a graph plotting nitrogen fixation levels measured by semi-solid based acetylene reduction assay (ARA) activity. K23E showed the highest level of nitrogenase activity, although M164 mutants also were able to show some derepression. FIG. 22B is a table listing the strains tested in FIG. 22A.

FIGS. 23A-23B show GFP expression levels of various strains in 3044 background. FIG. 23A is a graph plotting GFP expression levels measured by flow cytometry. Strains listed on the x-axis carry a plasmid that encodes a nifH-promoter driven GFP gene. PnifH was active under no nitrogen conditions in all edited strains. Under 10 mM NH₄Cl, the only SNP that showed significant PnifH activity was K21E. FIG. 23B is a table listing the strains tested in FIG. 23A.

FIGS. 24A-24B show nitrogen fixation levels in various strains. FIG. 24A is a graph plotting nitrogen fixation levels measured by ARA activity. K21E was the only mutation that showed significant derepression under 10 mM ammonium chloride. FIG. 24B is a table listing the strains tested in FIG. 24A.

FIGS. 25A-25B include a graph plotting ammonium production levels in nifA mutants in CI3044 (FIG. 25A), and a graph plotting growth measured by ending OD590 in the same nifA mutants (FIG. 25B).

FIG. 26A is a depiction of predicted domains of the NifA protein. FIG. 26B is a domain alignment of the central AAA+ domain of the known NifA proteins (SEQ ID NOs: 74-91). Walker A and Walker B domains are boxed. Potential arginine fingers (R321, R330, R342) are labeled with black circles.

FIG. 27A is an image of the AlphaFold predicted structure of CI1666 NifA protein. Lysine residues (*) and methionine residues (#) are indicated. FIG. 27B is a closeup image of the AlphaFold predicted structure, showing that the K23 does not bond with the neighboring residues since the distance between the two residues is larger than 3.3 Å. FIG. 27C is another image of the AlphaFold predicted structure, showing that K23 localizes in the same plane as the domain that includes conserved arginine fingers surrounded by other acidic residues (E325, E327, E329) as well as M164.

### DETAILED DESCRIPTION

Nitrogen fertilizer can be the largest operational expense on a farm and the biggest driver of higher yields in row crops like corn and wheat. Described herein are microbial products that can deliver renewable forms of nitrogen in non-leguminous crops. While some microbes (e.g., endophytes) have the genetics necessary for fixing nitrogen in pure culture, the fundamental technical challenge is that wild-type microbes of cereals and grasses stop fixing nitrogen in fertilized fields. The application of chemical fertilizers and residual nitrogen levels in field soils signal the microbe to shut down the biochemical pathway for nitrogen fixation. The genetically engineered microbes (e.g., bacteria) and compositions provided herein can fix nitrogen under both nitrogen limiting and non-nitrogen limiting conditions (e.g., in the presence of ammonia) as well as in the presence of oxygen (e.g., at least 0.5% oxygen). The genetically engineered microbes (e.g., bacteria) and compositions provided herein can produce ammonium. Accordingly, such genetically engineered microbes (e.g., bacteria) and compositions can be applied to a plant and used to increase the amount of atmospheric derived nitrogen in plants (e.g., non-leguminous plants such as barley, canola, corn, peanut, rice, sorghum, soybean, turfgrass and wheat), even in fertilized fields.

Microbes in and around food crops can influence the traits of those crops. Plant traits that can be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

One target for genetic modification to facilitate field-based nitrogen fixation using the methods described herein is the NifA protein. The NifA protein is an activator for expression of nitrogen fixation genes. Increasing the expression of NifA (either constitutively or during high ammonia condition) circumvents the native ammonia-sensing pathway. Reducing the production of NifL proteins, a known inhibitor of NifA, can also lead to an increased level of freely active NifA. Increasing the transcription level of the nifAL operon (either constitutively or during high ammonia condition) can also lead to an overall higher level of NifA proteins. An elevated level of nifAL expression can be achieved by altering the promoter itself or by reducing the expression of NtrB (part of ntrB and ntrC signaling cascade that originally would result in the shutoff of nifAL operon during high nitrogen condition). A high level of NifA achieved by these or any other methods described herein increases the nitrogen fixation activity of the endophytes.

The NifA protein is composed of an N-terminal GAF domain, an AAA+ ATPase domain, and a C-terminal DNA binding domain. The AAA+ domain interacts with the sigma54-RNA polymerase and hydrolyzes ATP. ATP hydrolysis is required for the formation of an open complex to initiate transcription (Bush et al., Microbiology and Molecular Biology Reviews. 2012; 76(3):497-529). The GAF domain senses ammonium status and controls ATPase activity.

As described herein, mutations (e.g., point mutations) can be made in the gene encoding the NifA protein that lead to constitutive nitrogenase expression. Such mutations can be generated by mutagenizing NifA and then selecting microbes that grow under nitrogenase repressing conditions (e.g., in the presence of oxygen or nitrogen). For example, as shown in Example 1, a bacterial strain that fixes nitrogen in the presence of nitrogen, such as nitrogen-rich conditions or in the presence of ammonium, and in the presence of oxygen (e.g., a Paraburkholderia, Azospirillum, or Herbaspirillum strain), can be developed by mutating one or more njfgenes, such as the nifA gene, and then screening for mutants with nitrogenase activity in the presence of nitrogen and oxygen. In some cases, a mutant library is generated using directed evolution approaches. Methods for screening for mutants can include the use of a fluorescence reporter operably linked to a gene activated by nitrogen and oxygen, such as the nifH promoter in the engineered strains. Alternatively, mutant libraries of a bacterial strain that fix nitrogen in the presence of nitrogen and oxygen can be made that by mutating residues of the nifA gene that were identified as highly conserved (See, for example, Example). In addition, the native or mutant nifA gene can be operably linked to a non-native promoter, such as a constitutively expressed promoter.

In some embodiments, the nifA gene encoding the NifA polypeptide can have one more genetic modifications. The one or more genetic modifications can help the engineered microbes to fix nitrogen in the presence of nitrogen, oxygen, ammonium, or a combination thereof. The one or more genetic modifications can increase transcriptional activation of nitrogen fixation genes or increase activity of proteins encoded by nitrogen fixation genes.

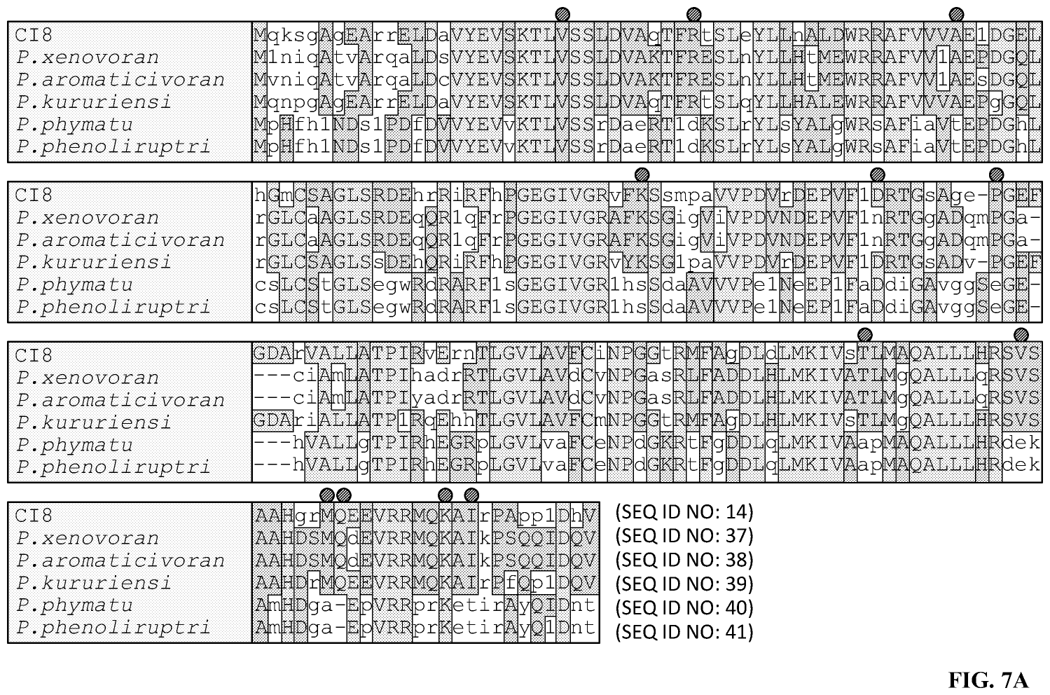

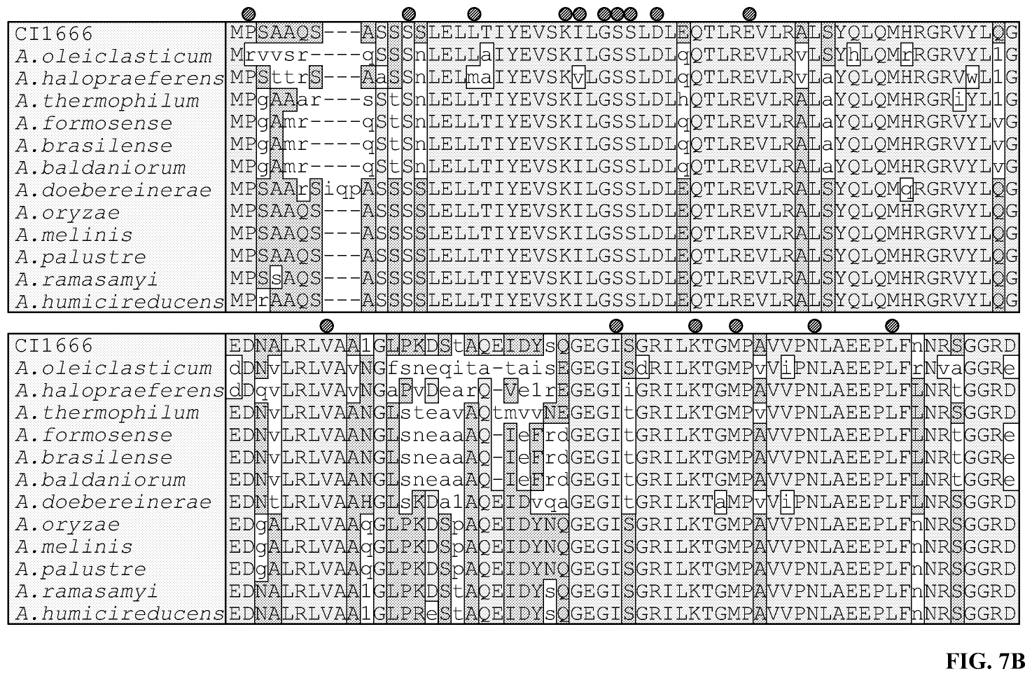

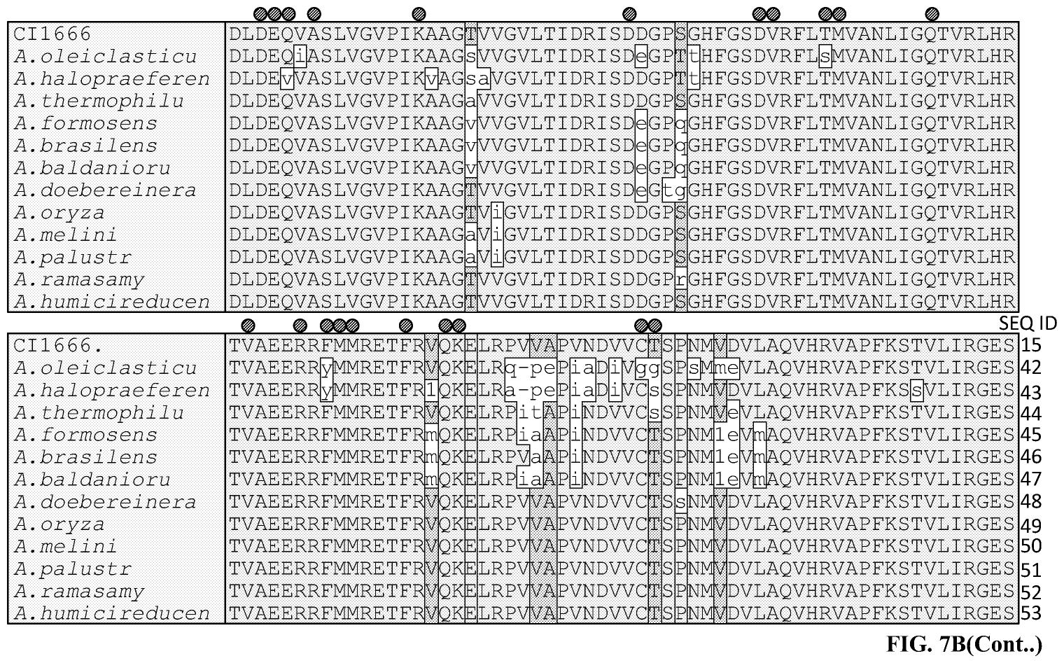

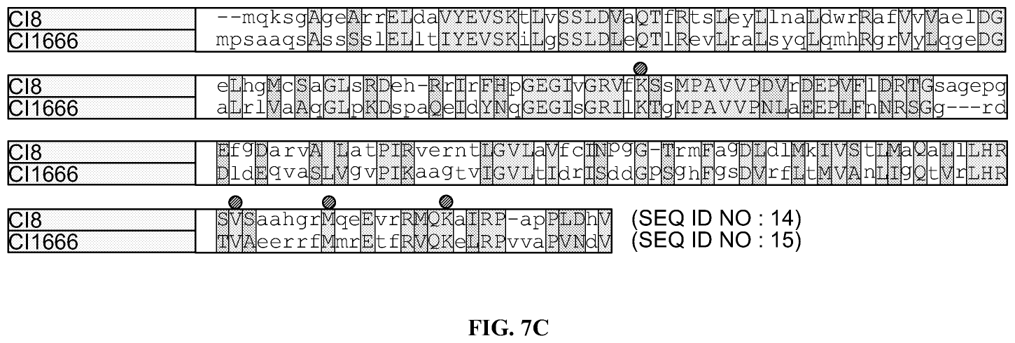

The one or more genetic modifications in nifA can result in a NifA polypeptide having a substitution at one or more amino acid positions corresponding to amino acids 7, 34, 42, 93, 108, 116, 122, 159, 166, 178, 185, 186, or 196 of SEQ ID NO: 14 or at one or more homologous positions in a homolog thereof. For example, a NifA polypeptide can have a substitution at two or more, three or more, four or more, five or more, six or more, seven or more, eight or more, nine or more, or ten or more amino acid positions corresponding to amino acids 7, 34, 42, 93, 108, 116, 122, 159, 166, 178, 185, 186, or 196 of SEQ ID NO: 14. FIG. 7A provides an alignment of the N-terminal region of the NifA polypeptide from Paraburkholderia tropicania (SEQ ID NO: 14) with homologs from P. xenovorans (SEQ ID NO: 37), P. aromaticivorans (SEQ ID NO: 38), P. kururiensis (SEQ ID NO: 39), P. phymatum (SEQ ID NO: 40), and P. phenoliruptrix (SEQ ID NO: 41).

In some embodiments, the genetic modification in a NifA polypeptide having a substitution is at an amino acid position that corresponds to amino acid 7 of SEQ ID NO: 14 or at a homologous position in a homolog thereof. For example, the amino acid at position 7 of SEQ ID NO: 14 or at a homologous position in a homolog thereof can be substituted with the amino acid D. In some embodiments, the genetic modification in a NifA polypeptide having a substitution at an amino acid position that corresponds to amino acid 34 of SEQ ID NO: 14 or at a homologous position in a homolog thereof. For example, the amino acid at position 34 of SEQ ID NO: 14 or at a homologous position in a homolog thereof can be substituted with the amino acid E. In some embodiments, the genetic modification in a NifA polypeptide having a substitution at an amino acid position that corresponds to amino acid 42 of SEQ ID NO: 14 or at a homologous position in a homolog thereof. For example, the amino acid at position 42 of SEQ ID NO: 14 or at a homologous position in a homolog thereof can be substituted with the amino acid D or S. In some embodiments, the genetic modification in a NifA polypeptide having a substitution at an amino acid position that corresponds to amino acid 93 of SEQ ID NO: 14 or at a homologous position in a homolog thereof. For example, the amino acid at position 93 of SEQ ID NO: 14 or at a homologous position in a homolog thereof can be substituted with the amino acid E or V. In some embodiments, the genetic modification in a NifA polypeptide having a substitution at an amino acid position that corresponds to amino acid 108 of SEQ ID NO: 14 or at a homologous position in a homolog thereof. For example, the amino acid at position 108 of SEQ ID NO: 14 or at a homologous position in a homolog thereof can be substituted with the amino acid E. In some embodiments, the genetic modification in a NifA polypeptide having a substitution at an amino acid position that corresponds to amino acid 116 of SEQ ID NO: 14 or at a homologous position in a homolog thereof. For example, the amino acid at position 116 of SEQ ID NO: 14 or at a homologous position in a homolog thereof can be substituted with the amino acid H. In some embodiments, the genetic modification in a NifA polypeptide having a substitution at an amino acid position that corresponds to amino acid 122 of SEQ ID NO: 14 or at a homologous position in a homolog thereof. For example, the amino acid at position 122 of SEQ ID NO: 14 or at a homologous position in a homolog thereof can be substituted with the amino acid E. In some embodiments, the genetic modification in a NifA polypeptide having a substitution at an amino acid position that corresponds to amino acid 159 of SEQ ID NO: 14 or at a homologous position in a homolog thereof. For example, the amino acid at position 159 of SEQ ID NO: 14 or at a homologous position in a homolog thereof can be substituted with the amino acid T. In some embodiments, the genetic modification in a NifA polypeptide having a substitution at an amino acid position that corresponds to amino acid 166 of SEQ ID NO: 14 or at a homologous position in a homolog thereof. For example, the amino acid at position 166 of SEQ ID NO: 14 or at a homologous position in a homolog thereof can be substituted with the amino acid A. In some embodiments, the genetic modification in a NifA polypeptide having a substitution at an amino acid position that corresponds to amino acid 178 of SEQ ID NO: 14 or at a homologous position in a homolog thereof. For example, the amino acid at position 178 of SEQ ID NO: 14 or at a homologous position in a homolog thereof can be substituted with the amino acid A. In some embodiments, the genetic modification in a NifA polypeptide having a substitution at an amino acid position that corresponds to amino acid 185 of SEQ ID NO: 14 or at a homologous position in a homolog thereof. For example, the amino acid at position 185 of SEQ ID NO: 14 or at a homologous position in a homolog thereof can be substituted with the amino acid T. In some embodiments, the genetic modification in a NifA polypeptide having a substitution at an amino acid position that corresponds to amino acid 186 of SEQ ID NO: 14 or at a homologous position in a homolog thereof. For example, the amino acid at position 186 of SEQ ID NO: 14 or at a homologous position in a homolog thereof can be substituted with the amino acid R. In some embodiments, the genetic modification in a NifA polypeptide having a substitution at an amino acid position that corresponds to amino acid 196 of SEQ ID NO: 14 or at a homologous position in a homolog thereof. For example, the amino acid at position 196 of SEQ ID NO: 14 or at a homologous position in a homolog thereof can be substituted with the amino acid V.

In some embodiments, the NifA polypeptide comprises two or more substitutions (e.g., three or more substitutions, four or more substitutions, or five or more substitutions). For example, in some embodiments, the one or more substitutions in a NifA polypeptide are at amino acid positions that correspond to 108, 159, 166, and 185 of SEQ ID NO: 14 or at a homologous amino acid position in a homolog thereof. In some embodiments, the one or more substitutions in a NifA polypeptide having a substitution at amino acid positions 42, 122, and 166 of SEQ ID NO: 14 or at a homologous amino acid positions in a homolog thereof. In some embodiments, the one or more substitutions in a NifA polypeptide having a substitution at amino acid positions 42 and 178 of SEQ ID NO: 14 or at a homologous amino acid positions in a homolog thereof. In some embodiments, the one or more substitutions in a NifA polypeptide having a substitution at amino acid positions 186 and 196 of SEQ ID NO: 14 or at homologous amino acid positions in a homolog thereof. In some embodiments, the one or more substitutions in a NifA polypeptide having a substitution at amino acid positions 7, 34, 93, 116, and 178 of SEQ ID NO: 14 or at homologous amino acid positions in a homolog thereof. Multiple mutations can lead to greater nitrogen fixation or increased ability to withstand nitrogen fixation repression in the presence of nitrogen or oxygen. For example, the nifA gene can encode a NifA polypeptide having a substitution at the following amino acids of SEQ ID NO: 14: a) D108E, D159T, T166A, and M185T; b) N42D, D122A, and T166A; c) N42S and V178A; d) Q186R and I196V; ore) G7D, R34E, M93V, P116H, and V178M.

In some embodiments, the substitution in the NifA polypeptide is D108E, D159T, T166A, and M185T of SEQ ID NO: 14 or homologous amino acids in a homolog thereof. In some embodiments, the substitution in the NifA polypeptide is N42D, D122A, and T166A or homologous amino acids in a homolog thereof. In some embodiments, the substitution in the NifA polypeptide is N42S and V178A or homologous amino acids in a homolog thereof. In some embodiments, the substitution in the NifA polypeptide is Q186R and I196V or homologous amino acids in a homolog thereof. In some embodiments, the substitution in the NifA polypeptide is G7D, R34E, M93V, P116H, and V178M or homologous amino acids in a homolog thereof.

In some embodiments, the NifA polypeptide encoded by the gene with one or more genetic modifications comprises a substitution at amino acids corresponding to S28P, M96T, and M164L of SEQ ID NO: 14 or homologous amino acid positions in a homolog thereof. In some embodiments, the NifA polypeptide encoded by the gene with one or more genetic modifications comprises a substitution at an amino acid corresponding to N42E of SEQ ID NO: 14 or at a homologous amino acid position in a homolog thereof.

In some embodiments, one or more genetic modifications in the nifA gene results in a NifA polypeptide that includes a substitution at one or more amino acid positions corresponding to amino acids 16, 23, 26, 28, 37, 65, 72, 93, 96, 123, 158, 164, 171, 183, or 209 of SEQ ID NO: 15 or at homologous amino acid positions in a homolog thereof.

In some embodiments, the genetic modification in the nifA gene results in a NifA polypeptide with a substitution at an amino acid position that corresponds to amino acid 16 of SEQ ID NO: 15 or at a homologous position in a homolog thereof. For example, the amino acid at position 16 of SEQ ID NO: 15 or at a homologous position in a homolog thereof can be substituted with the amino acid P. In some embodiments, the genetic modification in the nifA gene results in a NifA polypeptide with a substitution at an amino acid position that corresponds to amino acid 23 of SEQ ID NO: 15 or at a homologous position in a homolog thereof. For example, the amino acid at position 23 of SEQ ID NO: 15 or at a homologous position in a homolog thereof can be substituted with the amino acid E. In some embodiments, the genetic modification in the nifA gene results in a NifA polypeptide with a substitution at an amino acid position that corresponds to amino acid 26 of SEQ ID NO: 15 or at a homologous position in a homolog thereof. For example, the amino acid at position 26 of SEQ ID NO: 15 or a homologous position in a homolog thereof can be substituted with the amino acid E. In some embodiments, the genetic modification in the nifA gene results in a NifA polypeptide with a substitution at an amino acid position that corresponds to amino acid 28 of SEQ ID NO: 15 or a homologous position in a homolog thereof. For example, the amino acid at position 28 of SEQ ID NO: 15 or at a homologous position in a homolog thereof can be substituted with the amino acid P. In some embodiments, the genetic modification in the nifA gene results in a NifA polypeptide with a substitution at an amino acid position that corresponds to amino acid 37 of SEQ ID NO: 15 or at a homologous position in a homolog thereof. For example, the amino acid at position 37 of SEQ ID NO: 15 or at a homologous position in a homolog thereof can be substituted with the amino acid G. In some embodiments, the genetic modification in the nifA gene results in a NifA polypeptide with a substitution at an amino acid position that corresponds to amino acid 65 of SEQ ID NO: 15 or at a homologous position in a homolog thereof. For example, the amino acid at position 65 of SEQ ID NO: 15 or a homologous position in a homolog thereof can be substituted with the amino acid A. In some embodiments, the genetic modification in the nifA gene results in a NifA polypeptide with a substitution at an amino acid position that corresponds to amino acid 72 of SEQ ID NO: 15 or at a homologous position in a homolog thereof. For example, the amino acid at position 72 of SEQ ID NO: 15 or at a homologous position in a homolog thereof can be substituted with the amino acid E. In some embodiments, the genetic modification in the nifA gene results in a NifA polypeptide with a substitution at an amino acid position that corresponds to amino acid 93 of SEQ ID NO: 15 or at a homologous position in a homolog thereof. For example, the amino acid at position 93 of SEQ ID NO: 15 or at a homologous position in a homolog thereof can be substituted with the amino acid E or V. In some embodiments, the genetic modification in the nifA gene results in a NifA polypeptide with a substitution at an amino acid position that corresponds to amino acid 96 of SEQ ID NO: 15 or at a homologous position in a homolog thereof. For example, the amino acid at position 96 of SEQ ID NO: 15 or at a homologous position in a homolog thereof can be substituted with the amino acid T. In some embodiments, the genetic modification in the nifA gene results in a NifA polypeptide with a substitution at an amino acid position that corresponds to amino acid 123 of SEQ ID NO: 15 or at a homologous position in a homolog thereof. For example, the amino acid at position 123 of SEQ ID NO: 15 or at a homologous position in a homolog thereof can be substituted with the amino acid E. In some embodiments, the genetic modification in the nifA gene results in a NifA polypeptide with a substitution at an amino acid position that corresponds to amino acid 158 of SEQ ID NO: 15 or at a homologous position in a homolog thereof. For example, the amino acid at position 158 of SEQ ID NO: 15 or at a homologous position in a homolog thereof can be substituted with the amino acid N or T. In some embodiments, the genetic modification in the nifA gene results in a NifA polypeptide with a substitution at an amino acid position that corresponds to amino acid 164 of SEQ ID NO: 15 or at a homologous position in a homolog thereof. For example, the amino acid at position 164 of SEQ ID NO: 15 or at a homologous position in a homolog thereof can be substituted with the amino acid I, L or T. In some embodiments, the genetic modification in the nifA gene results in a NifA polypeptide with a substitution at an amino acid position that corresponds to amino acid 171 of SEQ ID NO: 15 or at a homologous position in a homolog thereof. For example, the amino acid at position 171 of SEQ ID NO: 15 or at a homologous position in a homolog thereof can be substituted with the amino acid K. In some embodiments, the genetic modification in the nifA gene results in a NifA polypeptide with a substitution at an amino acid position that corresponds to amino acid 183 of SEQ ID NO: 15 or at a homologous position in a homolog thereof. For example, the amino acid at position 183 of SEQ ID NO: 15 or at a homologous position in a homolog thereof can be substituted with the amino acid Q. In some embodiments, the genetic modification in the nifA gene results in a NifA polypeptide with a substitution at an amino acid position that corresponds to amino acid 209 of SEQ ID NO: 15 or at a homologous position in a homolog thereof. For example, the amino acid at position 209 of SEQ ID NO: 15 or at a homologous position in a homolog thereof can be substituted with the amino acid R.

In some embodiments, the substitution in the nifA gene results in a NifA polypeptide with a substitution at an amino acid position that corresponds to the following amino acids of SEQ ID NO: 15 or homologous amino acids in a homolog thereof: a) 37, 65, 93, 164, and 209; b) 16, 23, 72, 158, 171, and 183; c) 28, 96, and 164; d) 23, 148, and 164; e) 123 and 164; f26; org) 23.

In some embodiments, the substitution in the nifA gene results in a NifA polypeptide with a substitution at amino acid positions 37, 65, 93, 164, and 209 of SEQ ID NO: 15 or at homologous amino acid positions in a homolog thereof. In some embodiments, the substitution in the nifA gene results in a NifA polypeptide with a substitution at amino acid positions 16, 23, 72, 158, 171, and 183 of SEQ ID NO: 15 or at homologous amino acid positions in a homolog thereof. In some embodiments, the substitution in the nifA gene results in a NifA polypeptide with a substitution at amino acid positions 28, 96, and 164 of SEQ ID NO: 15 or at homologous amino acid positions in a homolog thereof. In some embodiments, the substitution in the nifA gene results in a NifA polypeptide with a substitution at amino acid positions 23, 148, and 164 of SEQ ID NO: 15 or at homologous amino acid positions in a homolog thereof. In some embodiments, the substitution in the nifA gene results in a NifA polypeptide with a substitution at amino acid positions 123 and 164 of SEQ ID NO: 15 or at homologous amino acid positions in a homolog thereof. In some embodiments, the substitution in the nifA gene results in a NifA polypeptide with a substitution at amino acid position 26 of SEQ ID NO: 15 or at homologous amino acid positions in a homolog thereof. In some embodiments, the substitution in the nifA gene results in a NifA polypeptide with a substitution at an amino acid position 23 of SEQ ID NO: 15 or at homologous amino acid positions in a homolog thereof. For example, in some embodiments, the substitution in nifA is at amino acids corresponding to the following amino acids of SEQ ID NO: 15 or at homologous amino acid positions in a homolog thereof: a) E37G, V65A, K93E, M164T, and C209R; b) L16P, K23E, K72E, D158N, Q171K, and R183Q; c) S28P, M96T, and M164L; d) K23E, D148G, and M164I; e) K123E and M164T; or f) G26E; or g) K23E.

In some embodiments, the substitution in the nifA gene results in a NifA polypeptide with the substitutions E37G, V65A, K93E, M164T, and C209R of SEQ ID NO: 15 or the same substitutions that correspond to homologous amino acids in a homolog thereof. In some embodiments, the substitution in the nifA gene results in a NifA polypeptide with the substitutions L16P, K23E, K72E, D158N, Q171K, and R183Q of SEQ ID NO: 15 or the same substitutions that correspond to homologous amino acids in a homolog thereof. In some embodiments, the substitution in nifA is S28P, M96T, and M164L of SEQ ID NO: 15 or the same same substitutions that correspond to homologous amino acids in a homolog thereof. In some embodiments, the substitution in the nifA gene results in a NifA polypeptide with the substitutions K23E, D148G, and M164I of SEQ ID NO: 15 or the same substitutions that corresponds to homologous amino acids in a homolog thereof. In some embodiments, the substitution in the nifA gene results in a NifA polypeptide with the substitution K123E and M164T of SEQ ID NO: 15 or the same substitutions that correspond to homologous amino acids in a homolog thereof. In some embodiments, the substitution in the nifA gene results in a NifA polypeptide with the substitution G26E of SEQ ID NO: 15 or the same substitution that corresponds to homologous amino acids in a homolog thereof. In some embodiments, the substitution in the nifA gene results in a NifA polypeptide with the substitution K23E of SEQ ID NO: 15 or the same substitution that corresponds to a homologous amino acid in a homolog thereof.

Genetic modifications of the NifA polypeptide can also include knockout mutations in which the entire nifA gene is deleted or deletions of a domain or a portion thereof of the nifA gene encoding the NifA polypeptide. The NifA polypeptide can include a deletion of amino acids corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200 or at a homologous amino acid position in a homolog thereof; or a deletion of the GAF domain of the NifA polypeptide.

For example, the NifA polypeptide can include a deletion of amino acids 2-23, 2-24, 2-51, 2-75, 2-105, 2-129, 2-139, 2-156, 2-167, 2-176, 2-202, or 2-252 of SEQ ID NO: 14, or at homologous amino acid positions in a homolog thereof.

In some embodiments, the NifA polypeptide can include a deletion of amino acids 186-196, 188-198, or 186-200 of SEQ ID NO: 14, or at homologous amino acid positions in a homolog thereof.

In some embodiments, NifA polypeptides can include a deletion of a domain or a portion thereof and also can include one or more amino acid substitutions. For example, the NifA polypeptide encoded by the gene with one or more genetic modifications can lack amino acids 188-198 of SEQ ID NO:1 (or homologous amino acids in a homolog thereof) and comprise a substitution at an amino acid corresponding to N42E of the SEQ ID NO: 14 or at a homologous amino acid position in a homolog thereof.

In some embodiments, the NifA polypeptide encoded by the gene with one or more genetic modifications exhibits increased transcriptional activation of nitrogen fixation genes in the presence of nitrogen and oxygen relative to that of a wild-type NifA polypeptide in the presence of nitrogen and oxygen.

Genetic modifications can also include insertions of a regulatory element. Regulatory elements include, for example, promoters, binding sites for enhancers or transcription factors, silencers (e.g. binding site for negative regulators), response elements, and terminator sites. In some embodiments, the regulatory element is a promoter. Regulatory elements can be native, exogenous, heterologous, or synthetic. Synthetic promoter are DNA sequences that do not exist in non-genetically modified organisms, but are a combinations of naturally occurring transcriptional elements that regulate the activity of a target genes. The regulatory elements can be constitutive or inducible. Regulator elements can be derived forma microbe of the same species as the engineered microbe (e.g. a native promoter). Regulatory elements can be derived from a microbe of the same genus as the engineered microbe. Regulatory elements can be derived from a microbe of a different species than the engineered microbe (e.g. exogenous promoter or heterologous promoter). Regulatory elements can be derived from a microbe of a different genus than the engineered microbe.

In some embodiments, the promoter is an acnB promoter (for example, SEQ ID NO: 16), a csp promoter, a gapA1 promoter (for example, SEQ ID NO: 17), a gltA promoter, a groS promoter (for example, SEQ ID NO: 18), an infC promoter (for example, SEQ ID NO: 6), an ompA promoter (for example, SEQ ID NO: 19), an oprF promoter (for example, SEQ ID NO: 20), apflB promoter (for example, SEQ ID NO: 21), a pgk2 promoter (for example, SEQ ID NO: 22), a ppsA promoter (for example, SEQ ID NO: 23), a rpl promoter, a rpmB promoter (for example, SEQ ID NO: 24), a rpoBC promoter (for example, SEQ ID NO: 25), a rps promoter, or a tufA-2 promoter (for example, SEQ ID NO: 26). In some embodiments, the csp promoter comprises a cspA3 promoter (for example, SEQ ID NO: 27), a cspA5 promoter (for example, SEQ ID NO: 28), a cspD promoter, a cspD-1 promoter, a cspD2 promoter (for example, SEQ ID NO: 29), or a cspJ promoter (for example, SEQ ID NO: 30). In some embodiments, the gltA promoter comprises a gitA1 promoter (for example, SEQ ID NO: 31) or a gltA2 promoter (for example, SEQ ID NO: 32). In some embodiments, the rps promoter comprises a rpsF promoter (for example, SEQ ID NO: 33) or a rpsL promoter (for example, SEQ ID NO: 34). In some embodiments, the rpl promoter comprises a rplL promoter (for example, SEQ ID NO: 35) or a rplMpromoter (for example, SEQ ID NO: 36). In some embodiments, the promoter is a synthetic promoter. In some embodiments, the synthetic promoter is a lil promoter (for example, SEQ ID NO: 3).

In some embodiments, the promoter has 85%, 90%, 95%, 99%, or 100% identity to SEQ ID Nos: 4-7 or 17-36.

In some embodiments, the promoter has 85%, 90%, 95%, 99%, or 100% identity to SEQ ID NO: 3.

In some embodiments, the coding sequence of the nifA gene can be inserted into a non-coding site of the genome of a genetically engineered bacterium described herein. In some embodiments, inserting the coding sequence of the nufA gene into a non-coding site of the genome of a genetically engineered bacterium results in expression of the nufA gene in nitrogen limiting and non-nitrogen limiting conditions. In some embodiments, the expression is constitutive. In some embodiments, the nifA gene is inserted into a non-coding region of the genome between two hypothetical genes that are transcribed in convergent fashion. In some embodiments, the coding sequence of the nifA gene and a promoter (e.g., any of the promoters described herein) are inserted into a non-coding site of the genome of a genetically engineered bacterium. For example, the promoter can be the cspE gene promoter (e.g., PcspE, also known as Prm1.2; SEQ ID NO: 4 and SEQ ID NO: 5). In some embodiments, the promoter has at least about 70%, about 75%, about 80%, about 85%, about 90%, about 95%, about 97%, about 98%, about 99%, or about 100% sequence identity to any one of SEQ ID NOs: 4-5.

In some embodiments, the genetically engineered microbes (e.g., bacteria) that have one more genetic modifications in a gene encoding a NifA polypeptide can include one or more additional genetic modifications in other genes that regulate nitrogen fixation or assimilation to, for example, increase nitrogen uptake in a plant or increase ammonium production. The additional genetic modifications in a gene regulating nitrogen fixation or assimilation can be in any of the genes comprising the nitrogen fixation and assimilation genetic regulatory network. In some embodiments, the nitrogen fixation and assimilation genetic regulatory network includes polynucleotides encoding genes and non-coding sequences that direct, modulate, and/or regulate microbial nitrogen fixation and/or assimilation and can comprise polynucleotide sequences of the nif cluster (e.g., nifB, nifC, . . . niZ), polynucleotides encoding nitrogen regulatory protein C, polynucleotides encoding nitrogen regulatory protein B, polynucleotide sequences of the gln cluster (e.g. glnA and glnD), draT, and ammonia transporters/permeases. In some cases, the nf cluster may comprise NifB, NifH, NifD, NifK, NifE, NifN, NifX, hesA, and NifV. In some cases, the Nif cluster may comprise a subset of NifB, NifH, NifD, NifK, NifE, NifN, NifX, hesA, and NifV.

In some embodiments, a trait that can be targeted for regulation by the methods described herein is nitrogen fixation. In some embodiments, a trait that can be targeted for regulation by the methods described herein is nitrogen assimilation. In some embodiments, a trait that can be targeted for regulation by the methods described herein is ammonium production.

In some embodiments, a trait that can be targeted for regulation by the methods described herein is colonization potential.

In some embodiments, in addition to at least one genetic modification to the nifA gene, there can be at least one modification in the gene regulating nitrogen fixation or assimilation can result in one or more of: constitutive expression of the nifA gene in nitrogen limiting and non-nitrogen limiting condition, activity of nifA in non-nitrogen limiting conditions, decreased uridylyl-transferase activity of GlnD, decreased adenylyl-removing activity of GlnE, and increased nitrogen excretion.

In some embodiments, in addition to containing at least one genetic modification to the nifA gene, genetically engineered bacteria as provided herein can comprise at least one modification in one or more genes regulating nitrogen fixation or assimilation selected from nifL, glnD, glnE, and ntrC.

In order to utilize elemental nitrogen (N) for chemical synthesis, life forms combine nitrogen gas (N₂) available in the atmosphere with hydrogen in a process known as nitrogen fixation. Diazotrophs (i.e., bacteria and archaea that fix atmospheric nitrogen gas) have evolved sophisticated and tight regulation of the nif gene cluster in response to environmental oxygen and available nitrogen. Nif genes encode enzymes involved in nitrogen fixation, such as the nitrogenase complex, and proteins that regulate nitrogen fixation. (See, e.g., Shamseldin 2013. Global J. Biotechnol. Biochem. 8(4):84-94), which discloses detailed descriptions of mf genes and their products, and is incorporated herein by reference. Described herein are methods of producing a plant with an improved trait comprising isolating bacteria from a first plant, introducing a genetic modification into a nif gene of the isolated bacteria, exposing a second plant to the variant bacteria, isolating bacteria from the second plant having an improved trait relative to the first plant, and repeating the steps with bacteria isolated from the second plant.

Changes to the transcriptional and post-translational levels of components of the nitrogen fixation regulatory network can be beneficial to the development of a microbe capable of fixing and transferring nitrogen to corn in the presence of fertilizer. To that end, described herein is Host-Microbe Evolution (HoME) technology, also referred to as directed evolution, which can precisely evolve regulatory networks and elicit novel phenotypes. In some embodiments, this technology enables precision evolution of the genetic regulatory network of microbes that actively fix nitrogen even in the presence of fertilizer in the field. In some embodiments, this technology enables precision evolution of the genetic regulatory network of microbes that exhibits increased transctiptional activation oc nitrogen fixation genes in the present of nitrogen and ozygen. In some embodiments, this technology enables precision evolution of microbes that overcome ammonium inhibition in the present of nitrogen. Also described herein are evaluations of the technical potential of evolving microbes that colonize corn root tissues and produce nitrogen for fertilized plants and evaluations of the compatibility of endophytes with standard formulation practices and diverse soils to determine feasibility of integrating the microbes into modern nitrogen management strategies.

In proteobacteria, regulation of nitrogen fixation centers on the σ54-dependent enhancer-binding protein NifA, the positive transcriptional regulator of the nif cluster. NifA upregulates the nif gene complex and drives nitrogen fixation when there is insufficient fixed nitrogen available to the microbe. NifL inhibits NifA when there is sufficient fixed N available to the microbe. Intracellular levels of active NifA are controlled by two key factors: transcription of the nifLA operon, and inhibition of NifA activity by protein-protein interaction with NifL. Both of these processes are responsive to intraceullar glutamine levels via the PII protein signaling cascade. This cascade is mediated by GlnD, which directly senses glutamine and catalyzes the uridylylation or deuridylylation of two PII regulatory proteins—GlnB and GlnK—in response the absence or presence, respectively, of bound glutamine. Under conditions of nitrogen excess, unmodified GlnB signals the deactivation of the nifLA promoter. However, under conditions of nitrogen limitation, GlnB is post-translationally modified, which inhibits its activity and leads to transcription of the nifLA operon. In this way, nifLA transcription is tightly controlled in response to environmental nitrogen via the PII protein signaling cascade. On the post-translational level of NifA regulation, GlnK inhibits the NifL/NifA interaction in a matter dependent on the overall level of free GlnK within the cell.

NifA is transcribed from the nfLA operon, whose promoter is activated by phosphorylated NtrC, another σ54-dependent regulator. The phosphorylation state of NtrC is mediated by the histidine kinase NtrB, which interacts with deuridylylated GlnB but not uridylylated GlnB. Under conditions of nitrogen excess, a high intracellular level of glutamine leads to deuridylylation of GlnB, which then interacts with NtrB to deactivate its phosphorylation activity and activate its phosphatase activity, resulting in dephosphorylation of NtrC and the deactivation of the nifLA promoter. However, under conditions of nitrogen limitation, a low level of intracellular glutamine results in uridylylation of GlnB, which inhibits its interaction with NtrB and allows the phosphorylation of NtrC and transcription of the nifLA operon. In this way, nifLA expression is tightly controlled in response to environmental nitrogen via the PII protein signaling cascade. nifA, ntrB, ntrC, and glnB, are all genes that can be mutated in the methods described herein. These processes can also be responsive to intracellular levels of ammonia, ammonium, urea or nitrates.

The activity of NifA is also regulated post-translationally in response to environmental nitrogen, most typically through NifL-mediated inhibition of NifA activity. In general, the interaction of NifL and NifA is influenced by the PII protein signaling cascade via GlnK, although the nature of the interactions between GlnK and NifL/NifA varies significantly between diazotrophs. In Klebsiella pneumoniae, both forms of GlnK inhibit the NifL/NifA interaction, and the interaction between GlnK and NifL/NifA is determined by the overall level of free GlnK within the cell. Under nitrogen-excess conditions, deuridylylated GlnK interacts with the ammonium transporter AmtB, which serves to both block ammonium uptake by AmtB and sequester GlnK to the membrane, allowing inhibition of NifA by NifL. On the other hand, in Azotobacter vinelandii, interaction with deuridylylated GlnK is required for the NifL/NifA interaction and NifA inhibition, while uridylylation of GlnK inhibits its interaction with NifL. In diazotrophs lacking the nifL gene, there is evidence that NifA activity is inhibited directly by interaction with the deuridylylated forms of both GlnK and GlnB under nitrogen-excess conditions. In some bacteria the Nif cluster can be regulated by glnR, which can comprise negative regulation. Regardless of the mechanism, post-translational inhibition of NifA is an important regulator of the nif cluster in most known diazotrophs. In some embodiments, one or more of nfL, amtB, glnK, and glnR can be mutated in the bacterial strains described herein.

Loss of NifL function can remove repression of NifA in nitrogen-limiting conditions. In some embodiments, at least one modification in a gene regulating nitrogen fixation or assimilation results in decreased expression of nfL. In some embodiments, at least one modification in a gene regulating nitrogen fixation or assimilation comprises a deletion of all or a portion of the coding sequence of the nifL gene. In some embodiments, in addition to at least one genetic modification to the nifA gene, there can be at least one modification in a gene regulating nitrogen fixation or assimilation comprises a deletion of a portion of the coding sequence of the nifL gene. For example, a middle portion of the coding sequence of the nifL gene can be deleted. In some embodiments, the first 30 base pairs and the last 83 base pairs of the nfL coding sequence can be retained and the remaining base pairs can be deleted. In some embodiments, the deleted portion of the nifL coding sequence is replaced by a promoter, e.g., any of the promoters as described herein. For example, the promoter can be the infC gene promoter (PinfC, SEQ ID NO: 6), the cspE gene promoter (SEQ ID NO: 4 and SEQ ID NO: 5), or the ompXgene promoter (Prm5; SEQ ID NO: 7). For additional promoters see International Publication No. WO/2019/084059, which is incorporated herein by reference in its entirety. In some embodiments, the promoter has at least about 70%, about 75%, about 80%, about 85%, about 90%, about 95%, about 97%, about 98%, about 99%, or about 100% sequence identity to any one of SEQ ID NOs: 4-7.

In addition to regulating the transcription of the nifgene cluster, many diazotrophs have evolved a mechanism for the direct post-translational modification and inhibition of the nitrogenase enzyme itself, known as nitrogenase shutoff. This is mediated by ADP-ribosylation of the Fe protein (NifH) under nitrogen-excess conditions, which disrupts its interaction with the MoFe protein complex (NifDK) and abolishes nitrogenase activity. DraT catalyzes the ADP-ribosylation of the Fe protein and shutoff of nitrogenase, while DraG catalyzes the removal of ADP-ribose and reactivation of nitrogenase. As with nifLA transcription and NifA inhibition, nitrogenase shutoff is also regulated via the PII protein signaling cascade. Under nitrogen-excess conditions, deuridylylated GlnB interacts with and activates DraT, while deuridylylated GlnK interacts with both DraG and AmtB to form a complex, sequestering DraG to the membrane. Under nitrogen-limiting conditions, the uridylylated forms of GlnB and GlnK do not interact with DraT and DraG, respectively, leading to the inactivation of DraT and the diffusion of DraG to the Fe protein, where it removes the ADP-ribose and activates nitrogenase. The methods described herein also contemplate introducing genetic modification into the nifL, nifH, nifD, nifK, glnK, glnD, glnE draT genes, or a combination thereof.

Another target for genetic modification to facilitate field-based nitrogen fixation using the methods described herein is the GlnD/GlnB/GlnK PII signaling cascade. The intracellular glutamine level is sensed through the GlnD/GlnB/GlnK PII signaling cascade. Active site mutations in GlnD that abolish the uridylyl-transferase activity of GlnD disrupt the nitrogen-sensing cascade. In addition, reduction of the GlnB concentration short circuits the glutamine-sensing cascade. These mutations “trick” the cells into perceiving a nitrogen-limited state, thereby increasing the nitrogen fixation level activity. These processes can also be responsive to intracellular or extracellular levels of ammonia, urea, or nitrates.

The amtB protein can also be a target for genetic modification to facilitate field-based nitrogen fixation using the methods described herein. Ammonia uptake from the environment can be reduced by decreasing the expression level of amtB gene encoding the AmtB protein. Without intracellular ammonia, the endophyte is not able to sense the high level of ammonia, preventing the down-regulation of nitrogen fixation genes. Any ammonia that manages to get into the intracellular compartment is converted into glutamine. Intracellular glutamine level is the major currency of nitrogen sensing. Decreasing the intracellular glutamine level can prevent the cells from sensing high ammonium levels in the environment. This effect can be achieved by increasing the expression level of glutaminase, an enzyme that converts glutamine into glutamate. In addition, intracellular glutamine can also be reduced by decreasing glutamine synthase (an enzyme that converts ammonia into glutamine). In diazotrophs, fixed ammonia is quickly assimilated into glutamine and glutamate to be used for cellular processes. Disruptions to ammonia assimilation can enable diversion of fixed nitrogen to be exported from the cell as ammonia. The fixed ammonia is predominantly assimilated into glutamine by glutamine synthetase (GS), encoded by glnA, and subsequently into glutamine by glutamine oxoglutarate aminotransferase (GOGAT). In some examples, glnS encodes a glutamine synthetase. GS is regulated post-translationally by GS adenylyl transferase (GlnE), a bi-functional enzyme encoded by glnE that catalyzes both the adenylylation and de-adenylylation of GS through activity of its adenylyl-transferase (AT) and adenylyl-removing (AR) domains, respectively. Under nitrogen limiting conditions, glnA is expressed, and GlnE's AR domain de-adenylylates GS, allowing it to be active. Under conditions of nitrogen excess, glnA expression is turned off, and GlnE's AT domain is activated allosterically by glutamine, causing the adenylylation and deactivation of GS.

In some embodiments, modification of glnE can increase ammonium excretion. In some embodiments, a conserved aspartate-amino acid-aspartate (DXD) motif on AR domain of glnE can be changed. In some embodiments, changing a conserved DXD residue on AR domain of glnE can be used to remove de-adenylylation activity from glnE. In some embodiments, a D residue can be replaced on a DXD motif in the AR region of glnE. In some embodiments, the replacement of a D residue on a DXD motif in the AR region of glnE can leave the GlnB binding site intact so as to allow for regulation of adenylation activity while decreasing or preventing AR activity. In some embodiments, strains that can be utilized in this process of increasing ammonium excretion can include, but are not limited to, Paraburkholderia spp., Azospirillium spp., Herbaspirillum spp., Rahnella aquatilis, Kosakonia sacchari, and Klebsiella variicola strains.

In some embodiments, at least one modification in a gene regulating nitrogen fixation or assimilation results in decreased adenylyl-removing activity of GlnE. In some embodiments, a modification in a gene regulating nitrogen fixation or assimilation comprises a deletion of a portion of the coding sequence of the glnE gene. For example, in some embodiments, 1290 base pairs following the ATG start codon of the glnE gene are deleted. In some embodiments, a deletion of a portion of the coding sequence of the glnE gene results in decreased adenylyl-removing activity of GlnE. In some embodiments, a modification in a gene regulating nitrogen fixation or assimilation results in a truncated GlnE protein lacking an adenylyl-removing (AR) domain. In some embodiments, the GlnE protein lacking the AR domain has a functional ATase domain

Furthermore, the draT gene can also be a target for genetic modification to facilitate field-based nitrogen fixation using the methods described herein. Once nitrogen fixing enzymes are produced by the cell, nitrogenase shut-off represents another level in which cell downregulates fixation activity in high nitrogen condition. This shut-off can be removed by decreasing the expression level of DraT.

Methods for imparting new microbial phenotypes can be performed at the transcriptional, translational, and post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme's active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a GTG, TTG, or CTG start codon, which results in reduction in translational activity of the coding region. Complete abolishment of expression can be done by knocking out (deleting) the coding region of a gene. Frameshifting the open reading frame (ORF) can result in a premature stop codon along the ORF, thereby creating a non-functional truncated product. Insertion of in-frame stop codons can also similarly create a non-functional truncated product. Addition of a degradation tag at the N or C terminal can also be done to reduce the effective concentration of a particular gene.

Expression level of the genes described herein can be achieved by using a stronger promoter. To ensure high promoter activity during high nitrogen level condition (or any other condition), a transcription profile of the whole genome in a high nitrogen level condition could be obtained and active promoters with a desired transcription level can be chosen from that dataset to replace the weak promoter. Weak start codons can be swapped out with an ATG start codon for better translation initiation efficiency. Weak ribosomal binding sites (RBS) can also be swapped out with a different RBS with higher translation initiation efficiency. In addition, site specific mutagenesis can also be performed to alter the activity of an enzyme.

Increasing the level of nitrogen fixation that occurs in a plant can lead to a reduction in the amount of chemical fertilizer needed for crop production and reduce greenhouse gas emissions (e.g., nitrous oxide).

Nitrogenases are enzymes responsible for catalyzing nitrogen fixation. There are three types of nitrogenase found in various nitrogen-fixing bacteria: molybdenum (Mo) nitrogenase, vanadium (V) nitrogenase, and iron-only (Fe) nitrogenase. Nitrogenases are two-component systems made up of Component I (also known as dinitrogenase) and Component II (also known as dinitrogenase reductase). Component I is a MoFe protein in molybdenum nitrogenase, a VFe protein in vanadium nitrogenase, and a Fe protein in iron-only nitrogenase. Component II is a Fe protein that contains an iron-sulfur (Fe—S) cluster.

In some embodiments, varying the supply of cofactors can result in an increase of nitrogen fixation. For example, increasing sulfur uptake can provide a larger pool of cofactors for nitrogenase enzymes, thus increasing the number of functional nitrogenase complexes. In some embodiments, sulfur uptake can be increased by upregulating sulfate transport genes. Some examples of sulfate transport genes can include, but are not limited to, cysPTWA, sbp, cysZK.

In some embodiments, varying the supply of cofactors can result in an increase in nitrogen fixation. For example, increasing molybdenum (Mo) uptake can increase the number of functional nitrogenase complexes. In some embodiments, Mo uptake can be increased by upregulating Mo transport genes. Examples of Mo transport genes can include, but are not limited to, modEBA, modEB and modA.

In some embodiments, cofactor supply can be affected by iron uptake. Iron uptake can be influenced by the tonB transport system. In some embodiments, influencing iron uptake can be achieved by upregulating tonB transport system genes. Some examples of tonB transport system genes can include, but are not limited to, tonB, and exbAB. In some embodiments, iron uptake can be influenced by siderophores which increase iron uptake in microbes and plants. In some embodiments, influencing iron uptake can be achieved by upregulating siderophore biosynthesis genes. Some examples of siderophore biosynthesis genes can include, but are not limited to, yhfA, yusV, sbnA, fiu, yfiZ, and fur.

Varying the metabolic flux to ATP can result in an increase of nitrogen fixation. For example, the metabolic flux to ATP can be increased by targeting glycogen biosynthesis. Glycogen biosynthesis can be influenced by shunting carbon to glycolysis, the TCA cycle and/or oxidative phosphorylation rather than glycogen synthesis. In some embodiments, glycogen biosynthesis can be influenced by deleting or downregulating the relevant gene for glycogen synthase. An example of a glycogen synthase gene can be, but is not limited to, glgA.

Varying the number of nitrogenase enzymes per cell can result in an increase in nitrogen fixation. For example, the number of nitrogenase enzymes per cell can be affected by nif derepression. Nif derepression can be achieved by constitutively signaling nitrogen starvation. In some embodiments, nif derepression can be achieved by deleting the UR-domain of relevant genes. An example of a gene which can be targeted to derepress nif genes can be, but is not limited to, glnD. In some embodiments, the transcription of the nif cluster(s) can be increased by inserting strong promoters upstream of a nifHDK or nifDK operon.

Another way to increase nitrogen fixation can be to increase the number of nitrogenase enzymes per cell by increasing nif cluster transcription. Nif cluster transcription can be increased by increasing nifA transcription. In some embodiments, nif cluster transcription can be influenced by increasing the copy number of a nifA gene in the genome.

Nif cluster transcription can also be increased by increasing NifA translation. In some embodiments, NifA translation can be increased by increasing the strength of the ribosome binding site in the nifA gene.

Altering the oxygen sensitivity of nitrogenase can result in an increase of nitrogen fixation. Oxygen sensitivity can be influenced by reducing oxygen sensing. In some embodiments, reducing oxygen sensing can be by disrupting oxygen-sensing genes. Some examples of oxygen-sensing genes can include, but are not limited to, nifT/fixU, fixJ and fixL.

In some embodiments, oxygen sensitivity can be influenced by keeping cytosolic oxygen levels low by promoting cytochrome bd-mediated respiration. In some embodiments, oxygen sensitivity can be influenced by upregulating genes encoding cytochrome bd oxidase and/or knocking out alternative cytochrome systems. Some examples of genes encoding cytochrome bd genes can include, but are not limited to, cydABX, cydAB, and cydX. In some embodiments, nitrogenase can be protected from oxidation by altering redox balance in the cell. Redox balance can be altered through ROS scavenging. One strategy for accomplishing ROS scavenging would be to upregulate relevant genes. Some examples of ROS scavenging genes can be, but are not limited, to grxABCD, trxA, trxC, and tpx.

In some embodiments, oxygen sensitivity can be influenced by scavenging free oxygen. In some embodiments, scavenging free oxygen can be achieved by upregulating bacterial hemoglobin genes.

An example of a hemoglobin gene can be, but is not limited to, glbN. In some embodiments, scavenging free oxygen can be achieved by upregulating fixNOPQ genes which code for a high-affinity heme-copper cbb3-type oxidase.

Modifying integration host factor a (IHFa) can result in an increase of nitrogenase expression. In some embodiments, nitrogenase expression can be increased by facilitating interaction between nifA and σ54 at the upstream activation sequence upstream of certain genes. In particular, upregulation of IHF can increase nitrogenase transcription. In some embodiments, upregulation of IHF in combination with nifA and σ54 can increase transcription of nitrogenase operon. In some embodiments, strains that can be utilized in this process of increasing nitrogen expression can include, but is not limited to, Rahnella aquatilis, Kosakonia sacchari, and/or Klebsiella variicola strains. In some embodiments, the upregulation of a nitrogenase operon can be more effective when stacked with mutation in a gene encoding σ54.

Modifying a gene encoding σ54 can result in an increase of nitrogenase expression. In some embodiments, upregulation of a gene for σ54 can increase nitrogenase transcription. An example of a gene encoding σ54 includes, but is not limited to, rpoN. In some embodiments, upregulation of σ54 in combination with nifA and IHF can increase transcription of a nitrogenase operon. In some embodiments, the transcription of the nitrogenase operon can be further improved by stacking the upregulation of σ54 with an IHF mutation.

In some embodiments, deleting a protein such as DraT in a strain can increase nitrogenase activity. In some embodiments, DraT can post-translationally modify a nitrogenase enzyme to inhibit its activity

In some embodiments, modification of an asnB gene can increase ammonium excretion. In particular, truncation and upregulation of an asnB gene can convert glutamine back to ammonium. The AsnB enzymes contain two domains; one can deaminate glutamine to release ammonium, and the other uses the ammonium to generate asparagine. Truncating AsnB to delete the asparagine synthase domain and/or upregulating the glutamine deaminase domain can help to convert back cellular glutamine to ammonium, thereby increasing ammonium excretion.

In some embodiments, modification of an asnB gene can increase ammonium excretion. In particular, deletion of an asnB gene can reduce ammonium sinks in a cell. asnB is able to use cytosolic ammonium instead of glutamine as an N donor. In some embodiments, deleting, truncating, or upregulating asnB can increase the amount of ammonium excreted from a cell.

The GlnD protein has four domains: an N-terminal uridyl-transferase (UTase) domain; a central uridyl-removal (UR) domain, and two C-terminal ACT domains. The UTase activity is localized to the N-terminal NT domain. This domain has a distinct amino acid residue pattern with conserved glycine (G) and aspartate (D) residues that are important for nucleotidyltransferase activity and binding of metal ions respectively. Most substitutions for conserved glycine and aspartate residues in this domain abolish glnD's UTase activity, preventing this enzyme from activating PII dependent nitrogen fixation and assimilation pathways. In some embodiments, modification of glnD can be beneficial in modifying regulation of nitrogen assimilation. In some embodiments, modifications of glnD can be used to optimize regulation of nitrogen assimilation pathways through the PII protein signaling pathway. The glnD gene encodes a bifunctional enzyme that can uridylate and deuridylate down-stream signaling proteins based on cell's nitrogen status. For example, the enzyme encoded by glnD modifies the PII proteins GlnK and GlnB. The GlnD enzyme reversibly uridylylates and de-uridylylates the PII proteins in conditions of nitrogen limitation and excess, respectively. The PII proteins confer signaling cascades to nitrogen metabolic pathways. Examples of nitrogen metabolism genes influenced by PII protein signaling include but are not limited to, glnA encoding glutamine synthetase, ntrB/glnL encoding sensory histidine kinase/phosphatase ntrB, glnG/ntrC DNA-binding transcriptional regulator ntrC and the nifLA operon. In some embodiments, glnD can be deleted so as to decrease the transcription of nitrogen assimilation genes and the amount of nitrogen assimilated within a cell. In some embodiments, glnD can be modified by deleting the ACT12 region, deleting the UR region and/or by deactivating the UR region by mutating specific amino acid residues (for example residues 90, 91 and/or 104).

In some embodiments, modification of glnD can be beneficial in increasing nitrogenase activity, ammonium excretion and/or plant growth. In particular, removal of a nitrogen sensing region can increase nitrogenase activity and/or plant growth. In some embodiments, an ACT domain of glnD can be deleted. In some embodiments, ACT domain is involved in sensing nitrogen status via allosteric regulation by glutamine. Removing an ACT domain can decrease uridylyl-transferase activity, thereby signaling nitrogen excess and downregulating nitrogen assimilation genes, leading to an increase in ammonium excretion. In some embodiments, strains that can be utilized in this process of increasing nitrogenase activity, ammonium excretion and/or plant growth can include, but are not limited to, Kosakonia sacchari and Klebsiella variicola strains.

In some embodiments, modification of glnD can be beneficial in increasing nitrogenase activity, ammonium excretion and/or plant growth. In particular, removal or deactivation of an uridylyl-transferase (UT) region within a domain of glnD can increase nitrogenase activity, ammonium excretion and/or plant growth. Removing or deactivating a UT domain can decrease uridylyl-transferase activity, thereby signaling nitrogen excess and downregulating nitrogen assimilation genes, leading to an increase in ammonium excretion.

In some embodiments, at least one modification in a gene regulating nitrogen fixation or assimilation comprises a deletion of all or a portion of the coding sequence of the glnD gene. In some embodiments, the at least one modification in a gene regulating nitrogen fixation or assimilation comprises a deletion of the N-terminal GlnD-UTase domain. For example, the NT GlnD-UTase domain can be deleted by removing 975 nucleotides after the start codon. In some embodiments, at least one modification in a gene regulating nitrogen fixation or assimilation comprises a deletion of all of the coding sequence of the glnD gene. For example, all 2,676 nucleotides of the glnD gene can be deleted from the genome of the genetically engineered bacteria. In some embodiments, the at least one modification in a gene regulating nitrogen fixation or assimilation comprises at least one point mutation in the coding sequence of the glnD gene. In some embodiments, the coding sequence of the glnD gene comprising at least one point mutation encodes a GlnD protein with amino acid substitutions comprising G90L, G91D, and D104A. In some embodiments, seven point mutations are incorporated into the glnD gene sequence (SEQ ID NO: 8) to encode the following amino-acid changes: G90L, G91D, D104A in the UTase domain. See Table 8 for GlnD sequences described above.

In some embodiments, modification of GlnB can be beneficial in increasing nitrogen compound excretion. In some embodiments, the uridylyl transferase (UTase) domain of GlnD modifies GlnB at tyrosine-51. In some embodiments, by modifying the UTase domain of GlnD, GlnB-UMP production can be decreased. In some embodiments, by removing the UTase domain of GlnD, GlnB-UMP production can be decreased. In some embodiments, by changing the UTase domain of GlnD, GlnB-UMP production can be prevented. In some embodiments, by removing the UTase domain of GlnD, GlnB-UMP production can be prevented. In some embodiments, GlnB can be modified by deleting tyrosine-51. In some embodiments, GlnB can be modified by modifying GlnB at tyrosine-51.

In some embodiments, modification of GlnK can be beneficial in increasing ammonium excretion. In some embodiments, GlnK can behave within a strain as a GlnB analogue based on a similarity of structure between GlnK and GlnB. In some embodiments, modifying GlnK can increase ammonium excretion by removing inhibitory effects that can be based on GlnK. In some embodiments, by changing GlnK, inhibitory effects on ammonium excretion can be decreased. In some embodiments, by removing GlnK, inhibitory effects on ammonium excretion can be decreased. In some embodiments, by changing GlnK, inhibitory effects on ammonium excretion based on GlnK can be prevented. In some embodiments, by removing the glnK gene, inhibitory effects on ammonium excretion based on GlnK can be prevented.

In some embodiments, modification of glnK can be beneficial in increasing ammonium excretion. In some embodiments, the UTase domain of GlnD modifies glnK at tyrosine-51. In some embodiments, by modifying the UTase domain of GlnD, glnK-UMP production can be decreased. In some embodiments, by removing the UTase domain of GlnD, glnK-UMP production can be decreased. In some embodiments, by changing the UTase domain of GlnD, glnK-UMP production can be prevented. In some embodiments, by removing the UTase domain of GlnD, glnK-UMP production can be prevented. In some embodiments, GlnK can be modified by deleting tyrosine-51. In some embodiments, GlnK can be modified by modifying GlnK at tyrosine-51.

In some embodiments, modification of the glnL encoding the NtrB protein can be beneficial in increasing ammonium excretion. In particular, modification of NtrB can be beneficial in controlling glnA transcription independent of nitrogen status. In some embodiments, modification of ntrB can be achieved by deleting specific resides to titrate activity.

In some embodiments, modification of glnA can be beneficial in increasing ammonium excretion. In some embodiments, modification of NtrC can be beneficial in modifying the level of GlnA protein in the cell. NtrC is the member of the two-component regulatory system NtrB/NtrC, which controls expression of the nitrogen-regulated (ntr) genes in response to nitrogen limitation. Under nitrogen limited conditions, PII signaling proteins initiate a phosphorylation cascade that leads to the phosphorylation of the aspartate (D54) residue of NtrC. The phosphorylated form of NtrC binds upstream of multiple nitrogen metabolism genes it regulates and activates their transcription. Changing aspartate residue to a more negatively charged amino acid residue, glutamate (D54E), led NtrC to behave like phosphorylated and constitutively activated the transcription of its downstream target genes (Klose et al., J Mol Biol., 232(1):67-78, 1993). On the other hand, changing aspartate to alanine (D54A), prohibited phosphorylation of this residue, and hence activation of NtrC, resulting in lack of transcriptional response even under nitrogen limited conditions. In some embodiments, modification of NtrC can be beneficial by preventing the phosphorylization of NtrC. Phosphorylated NtrC can lead to transcriptional activation of glnA. As such, modification of ntrC so as to prevent the phosphorylization of ntrC can be beneficial in decreasing transcription of glnA. In some embodiments, modification of NtrC can be achieved by replacing asparate 54.

In some embodiments of the genetically engineered bacteria described herein, the NtrC binding site upstream of nifA is replaced by a constitutive promoter. This can remove NtrC for transcriptional activation of nifA. In some embodiments, the at least one modification in a gene regulating nitrogen fixation or assimilation comprises a mutation in the coding sequence of the ntrC gene. In some embodiments, at least one modification in a gene regulating nitrogen fixation or assimilation comprises changing the 161st nucleotide of the ntrC coding sequence from A to C (SEQ ID NO: 11). In some embodiments, the mutation in the coding sequence of the ntrC gene encode NtrC protein comprising a D54A amino acid substitution In some embodiments, modification of Glutaminase B can be beneficial in increasing ammonium excretion. In some embodiments, the conversion of glutamine back to glutamate and ammonia by Glutaminase B can be upregulated so as to increase ammonium excretion.

In some embodiments, modification of a nitrogenase operon can be beneficial in increasing nitrogenase expression. In some embodiments, it can be beneficial to upregulate nitrogenase operons so as to increase nitrogenase transcription. In some embodiments, promoters from within the bacterium that are active when the bacterium is colonizing the rhizosphere can be inserted in front of nitrogenase operons to upregulate nitrogenase operons. In some embodiments, nifL can be deleted within nitrogenase operons to upregulate nitrogenase operons. In some embodiments, nifL can be deleted within nitrogenase operons to upregulate nitrogenase operons. In some embodiments, multiple promoters can be placed directly in front of nfHDK genes so as to circumvent nifA transcription control. In some embodiments, strains that can be utilized in this process of increasing nitrogenase expression can include, but are not limited to, Rahnella aquatilis and Klebsiella variicola strains.

In some embodiments, modification of glnE can be beneficial in increasing ammonium excretion. In some embodiments, a conserved aspartate-amino acid-aspartate (DXD) motif on AR domain of glnE can be changed. In some embodiments, changing a conserved DXD residue on AR domain of glnE can be used to remove de-adenylylation activity from glnE. In some embodiments, a D residue can be replaced on a DXD motif in the AR region of glnE. In some embodiments, the replacement of a D residue on a DXD motif in the AR region of glnE can leave the GlnB binding site intact so as to allow for regulation of adenylation activity while decreasing or preventing AR activity.

In some embodiments, modification of glnA can be beneficial in increasing AMM excretion. In some embodiments, glnA can be downregulated by inserting the promoters of glnB, glnD, and/or glnE upstream of the glnA gene. In some embodiments, modification of glnA can decouple glnA expression from an N-status signaling cascade and decrease expression to a basal level so that more fixed nitrogen remains unassimilated.

In some embodiments, modification of GOGAT can be beneficial in increasing ammonium excretion. In some embodiments, GOGAT can be downregulated by inserting upstream of the GOGAT genes a promoter that controls glnB, glnD, and/or glnE. Downregulation of GOGAT can, in turn, lead to lowering glutamine oxyglutarate aminotransferase expression. In some embodiments, modification of GOGAT can decouple GOGAT expression from an N-status signaling cascade and decrease expression to a basal level so that more fixed nitrogen remains unassimilated.

In some embodiments, modification of GDH can be beneficial in increasing ammonium excretion. In some embodiments, GDH can be downregulated by inserting upstream of the GDH gene a promoter that controls glnB, glnD, and/or glnE. Downregulation of GDH can, in turn, lead to lowering NAD-specific glutamate dehydrogenase expression. In some embodiments, modification of GDH can decouple GDH expression from an N-status signaling cascade and decrease expression to a basal level so that more fixed nitrogen remains unassimilated.

In some embodiments, the amount of nitrogen provided to a microbe-associated plant is increased by decreasing the nitrogen assimilation in the microbe. Assimilation can be influenced by the excretion rate of ammonia. By targeting the assimilation of ammonia, nitrogen availability can be increased. In some embodiments, ammonia assimilation is influenced by decreasing the rate of ammonia reuptake after excretion. To decrease the rate of ammonia reuptake after excretion, any relevant gene can be knocked out. An example of an ammonia reuptake genes can be, but is not limited to, amtB.

In some embodiments, the assimilation can be influenced by the plant uptake rate. By targeting the plant nitrogen assimilation genes and pathways, nitrogen availability can be increased. In some embodiments, ammonia assimilation by a plant can be altered through inoculation with N-fixing plant growth promoting microbes. A screen can be carried out to identify microbes which induce ammonia assimilation in plants.

Although some endophytes have the ability to fix nitrogen in vitro, genes associated with nitrogen fixation can be silenced in the field by high levels of exogenous chemical fertilizers. The sensing of exogenous nitrogen can be decoupled from expression of the nitrogenase enzyme to facilitate field-based nitrogen fixation. Improving the integral of nitrogenase activity across time further serves to augment the production of nitrogen for utilization by the crop. Specific targets for genetic modification to facilitate field-based nitrogen fixation using the methods described herein include one or more genes selected from the group consisting of nifA, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, nifM, nifF, nifB, and nifQ.

Increasing the colonization capacity of the microbes can increase the amount of fixed nitrogen provided to a plant. The colonization can be influenced by altering carrying capacity (the abundance of microbes on the root surface) and/or microbe fitness. In some embodiments, influencing carrying capacity and microbe fitness can be achieved through altering organic acid transport. Organic acid transport can be improved by upregulating relevant genes. An example of an organic acid transport gene can be, but is not limited to, dctA.

For example, the colonization capacity can be affected by expression of agglutinins. Increased expression of agglutinins can help the microbes stick to plant roots. Examples of agglutinin genes can include, but are not limited to, fhaB and fhaC.

The colonization capacity can be affected by an increase in endophytic entry. For example, endophytic entry can be affected by plant cell wall-degrading enzymes (CDWE). Increasing CDWE expression and/or secretion can increase the colonization and endophytic entry of the microbes. Some examples of CDWEs are, but are not limited to, polygalacturonases and cellulases. An example of a polygalacturonases gene is pehA. In some embodiments, export of polygalacturonases and cellulases can be increased by providing an export signal with the enzymes.

Varying the carrying capacity can result in an increased amount of nitrogen being provided to an associated plant. Carrying capacity can be affected by biofilm formation. In some embodiments, carrying capacity can be affected by small RNA rsmZ. Small RNA rsmZ is a negative regulator of biofilm formation. In some embodiments, biofilm formation can be promoted by deleting or downregulating rsmZ, leading to increased translation of rsmA (a positive regulator of secondary metabolism) and biofilm formation.

In some embodiments, biofilm formation can be influenced by enhancing the ability of strains to adhere to the root surface. In some embodiments, biofilm formation can be promoted by upregulating large adhesion proteins. An example of a large adhesion protein can be, but is not limited to, lapA.

In some embodiments, carrying capacity can be affected by quorum sensing. In some embodiments, quorum sensing can be enhanced by increasing the copy number of AHL biosynthesis genes.

In some embodiments, the colonization of the rhizosphere can be influenced by root mass. For example, root mass can be affected by microbial IAA biosynthesis. Increased IAA biosynthesis by the microbe can stimulate root biomass formation. In some embodiments, influencing IAA biosynthesis can be achieved through upregulation (at a range of levels) of IAA biosynthesis genes. An example of an IAA biosynthesis gene can be, but is not limited to, ipdC.

In some embodiments ethylene signaling can induce systemic resistance in the plant and affect the colonization capacity of the microbe. Ethylene is a plant signaling molecule that elicits a wide range of responses based on plant tissue and ethylene level. The prevailing model for root ethylene response is that plants that are exposed to stress quickly respond by producing a small peak of ethylene that initiates a protective response by the plant, for example, transcription of genes encoding defensive proteins. If the stress persists or is intense, a second much larger peak of ethylene occurs, often several days later. This second ethylene peak induces processes such as senescence, chlorosis, and abscission that can lead to a significant inhibition of plant growth and survival. In some embodiments, plant growth promoting bacteria can stimulate root growth by producing the auxin IAA, which stimulates a small ethylene response in the roots. At the same time, the bacteria can prevent the second large ethylene peak by producing an enzyme (ACC deaminase) that slows ethylene production in the plant, thus maintaining an ethylene level that's conducive to stimulating root growth. Induction of systemic resistance in the plant can be influenced by bacterial IAAs. In some embodiments, stimulating IAAbiosynthesis can be achieved through upregulation (at a range of levels) of IAA biosynthesis genes. An example of a biosynthesis gene can be, but is not limited to, ipdC.

In some embodiments, colonization can be affected by ACC Deaminase. ACC Deaminase can be decrease ethylene production in the root by shunting ACC to a side product. In some embodiments, influencing ACC Deaminase can be achieved through upregulation of ACC Deaminase genes. Some examples of ACC Deaminase genes can include, but are not limited to, dcyD.

In some embodiments, the colonization can be influenced by carrying capacity and/or microbe fitness. For example, carrying capacity and/or microbe fitness can be affected by trehalose overproduction. Trehalose overproduction can increase of drought tolerance. In some embodiments, influencing trehalose overproduction can be achieved through upregulation (at a range of levels) of trehalose biosynthesis genes. Some examples of trehalose biosynthesis genes can include, but are not limited to, otsA, otsB, treZ and treY In some embodiments, upregulation of otsB can also increase nitrogen fixation activity.

In some embodiments, carrying capacity can be affected by root attachment. Root attachment can be influenced by exopolysaccharide secretion. In some embodiments, influencing exopolysaccharide secretion can be achieved through upregulation of exopolysaccharide production proteins. Some examples of exopolysaccharide production proteins can include, but are not limited to, yjbE and pssM. In some embodiments, influencing exopolysaccharide secretion can be achieved through upregulation of cellulose biosynthesis. Some examples of cellulose biosynthesis genes can include, but are not limited to, acs genes, and bcs gene clusters.

In some embodiments, carrying capacity and/or the microbe's fitness can be affected by fungal inhibition. Fungal inhibition can be influenced by chitinases which can break down fungal cell walls and can lead to biocontrol of rhizosphere fungi. In some embodiments, influencing fungal inhibition can be achieved through upregulation of chitinase genes. Some examples of chitinase genes can include, but are not limited to, chitinase class 1 and chiA.

In some embodiments, efficient iron uptake can help microbes to survive in the rhizosphere where they have to compete with other soil microbes and the plant for iron uptake. In some embodiments, high-affinity chelation (siderophores) and transport systems can help with rhizosphere competency by 1) ensuring the microbes obtains enough iron and 2) reducing the iron pool for competing species. Increasing the microbe's ability to do this could increase its competitive fitness in the rhizosphere. In some embodiments, influencing iron uptake can be by upregulating siderophore genes. Some examples of siderophore genes can include, but are not limited to, yhfA, yusV, sbnA, fiu, yfiZ, and fur. In some embodiments iron uptake can be influenced by the tonB transport system. In some embodiments, influencing iron uptake can be by upregulating tonB transport system genes. Some examples of tonB transport system genes can include, but are not limited to, tonB, and exbAB.

In some embodiments, carrying capacity and/or microbe fitness can be affected by redox balance and/or ROS scavenging. Redox balance and/or ROS scavenging can be influenced by bacterial glutathione (GSH) biosynthesis. In some embodiments, influencing bacterial glutathione (GSH) biosynthesis can be through upregulation of bacterial glutathione biosynthesis genes. Some examples of bacterial glutathione biosynthesis genes can include, but are not limited to, gshA, gshAB, and gshB.

In some embodiments, Redox balance can be influenced by ROS scavenging. In some embodiments, influencing ROS scavenging can be through upregulation of catalases. Some examples of catalases genes can include, but are not limited to, katEG, and Mn catalase.

In some embodiments, biofilm formation can be influenced by phosphorus signaling. In some embodiments, influencing phosphorus signaling can be by altering the expression of phosphorous signaling genes. Some examples of phosphorous signaling genes can include, but are not limited to, phoR and phoB.

In some embodiments, carrying capacity can be affected by root attachment. Root attachment can be influenced by surfactin biosynthesis. In some embodiments, influencing surfactin biosynthesis can be achieved by upregulating surfactin biosynthesis to improve biofilm formation. An example of surfactin biosynthesis genes can be, but is not limited to, srfAA.

In some embodiments, the colonization and/or microbe fitness can be influenced by carrying capacity, competition with other microbes and/or crop protection from other microbes. In some embodiments, competition with other microbes and/or crop protection from other microbes can be influenced by quorum sensing and/or quorum quenching. Quorum quenching can influence colonization by inhibiting quorum-sensing of potential pathogenic/competing bacteria. In some embodiments, influencing quorum quenching can be achieved by inserting and/or upregulating genes encoding quorum quenching enzymes. Some examples of quorum quenching genes can include, but are not limited to, ahlD, Y2-aiiA, aiiA, ytnP and attM. In some embodiments, modification of enzymes involved in quorum quenching, such as Y2-aiiA and/or ytnP can be beneficial for colonization. In some embodiments, upregulation of Y2-aiiA and/or ytnP can result in hydrolysis of extracellular acyl-homoserine lactone (AHL). aiiA is an N-acyl homoserine lactonase that is an enzyme that breaks down homoserine lactone. Breaking down AHL can stop or slow the quorum signaling ability of competing gram negative bacteria.

In some embodiments, carrying capacity and/or microbes fitness can be affected by rhizobitoxine biosynthesis. Rhizobitoxine biosynthesis can decrease ethylene production in the root by inhibiting ACC synthase. In some embodiments, influencing rhizobitoxine biosynthesis can be achieved by upregulating rhizobitoxine biosynthesis genes.

In some embodiments, carrying capacity can be affected by root attachment. Root attachment can be influenced by exopolysaccharide secretion. In some embodiments, influencing exopolysaccharide secretion can be achieved by generating hypermucoid mutants by deleting mucA.

In some embodiments, root attachment can be influenced by phenazine biosynthesis. In some embodiments, influencing phenazine biosynthesis can be achieved by upregulating phenazine biosynthesis genes to improve biofilm formation.

In some embodiments, root attachment can be influenced by cyclic lipopeptide (CLP) biosynthesis. In some embodiments, influencing cyclic lipopeptide (CLP) biosynthesis can be achieved by upregulating CLP biosynthesis to improve biofilm formation.

In some embodiments, carrying capacity and/or competition can be affected by antibiotic synthesis. Antibiotic synthesis can increase antibiotic production to kill competing microbes. In some embodiments, increasing antibiotic production can be achieved by mining genomes for antibiotic biosynthesis pathways and upregulation.

In some embodiments, colonization can be affected by desiccation tolerance. In some embodiments, modification of rpoE can be beneficial for colonization. In some embodiments, upregulation of rpoE can result in increasing expression of stress tolerance genes and pathways. In some embodiments, rpoE can be upregulated using a unique switchable promoter. In some embodiments, rpoE can be upregulated using an arabinose promoter. rpoE is a sigma factor similar to phyR. When expressed, rpoE can cause upregulation of multiple stress tolerance genes. As stress tolerance enzymes may not be useful during a colonization cycle, a switchable promoter can be used. In some embodiments, the promoter can be active during biomass growth and/or during seed coating. In some embodiments, a switchable promoter can be used where the sugar or chemical can be spiked in during the log phase of biomass growth but can also have the promoter not turned on during one or more other applications of the microbe. In some embodiments, rpoE can be upregulated while also downregulating rseA.

In some embodiments, colonization can be affected by desiccation tolerance. In some embodiments, modification of rseA can be beneficial for colonization. In some embodiments, rseA can be downregulated using a unique switchable promoter. In some embodiments, rseA can be downregulated using an arabinose promoter. rseA is an anti-sigma factor coexpressed with rpoE. In some embodiments, the enzymes remain bound to each other, which can decrease or disable rpoE's ability to act as a transcription factor. However, during stress conditions, resA can be cleaved and rpoE can be free to up/down regulate stress tolerance genes. By breaking co-transcription with rpoE, levels of rpoE and resA can be titered independently, which can be beneficial in optimizing colonization of engineered strains.

In some embodiments, a trait that can be targeted for regulation by the methods described herein is colonization potential. Accordingly, in some embodiments, pathways and genes involved in colonization can act as a target for genetic engineering and optimization.

In some cases, exopolysaccharides can be involved in bacterial colonization of plants. In some cases, plant colonizing microbes can produce a biofilm. In some cases, plant colonizing microbes secrete molecules which can assist in adhesion to the plant, or in evading a plant immune response. In some cases, plant colonizing microbes can excrete signaling molecules which alter the plants response to the microbes. In some cases, plant colonizing microbes can secrete molecules which alter the local microenvironment. In some cases, a plant colonizing microbe can alter expression of genes to adapt to a plant said microbe is in proximity to. In some cases, a plant colonizing microbe can detect the presence of a plant in the local environment and can change expression of genes in response.

In some embodiments, to improve colonization, a gene involved in a pathway selected from the group consisting of: exopolysaccharide production, endo-polygalaturonase production, trehalose production, and glutamine conversion can be targeted for genetic engineering and optimization.

In some embodiments, an enzyme or pathway involved in production of exopolysaccharides can be genetically modified to improve colonization. Exemplary genes encoding an exopolysaccharide producing enzyme that can be targeted to improve colonization include, but are not limited to, bcsii, bcsiii, andyjbE.

In some embodiments, an enzyme or pathway involved in production of a filamentous hemagglutinin can be genetically modified to improve colonization. For example, ajhaB gene encoding a filamentous hemagglutinin can be targeted to improve colonization.

In some embodiments, an enzyme or pathway involved in production of an endo-polygalaturonase can be genetically modified to improve colonization. For example, a pehA gene encoding an endo-polygalaturonase precursor can be targeted to improve colonization.

In some embodiments, an enzyme or pathway involved in production of trehalose can be genetically modified to improve colonization. Exemplary genes encoding a trehalose producing enzyme that can be targeted to improve colonization include, but are not limited to, otsB and treZ.

In some embodiments, an enzyme or pathway involved in conversion of glutamine can be genetically modified to improve colonization. For example, the glsA2 gene encodes a glutaminase which converts glutamine into ammonium and glutamate. Upregulating glsA2 improves fitness by increasing the cell's glutamate pool, thereby increasing available N to the cells. Accordingly, in some embodiments, the glsA2 gene can be targeted to improve colonization.

In some embodiments, colonization genes selected from the group consisting of: bcsii, bcsiii, yjbE, jhaB, pehA, otsB, treZ, glsA2, and combinations thereof, can be genetically modified to improve colonization.

Colonization genes that can be targeted to improve the colonization potential are also described in WO/2019/032926, which is incorporated by reference herein in its entirety.

### Methods of Use

Also provided herein are methods of increasing nitrogen fixation in a plant, comprising exposing the plant, a part of the plant, or soil into which the plant is planted or will be planted to bacteria comprising one or more genetic modifications introduced into one or more genes regulating nitrogen fixation. Also provided herein are methods of increasing the amount of atmospheric derived nitrogen in a plant, the method comprising contacting the plant, a part of the plant, or soil into which the plant is planted with a plurality of any of the genetically engineered bacteria described herein.

In some embodiments, provided herein are methods of increasing the total space on the root surface of a plant occupied by bacteria that can fix nitrogen in the presence of nitrogen, the method comprising contacting the plant, a part of the plant, or soil into which the plant is planted with a plurality of any of the genetically engineered bacteria described herein.

In some embodiments, the amount of atmospheric derived nitrogen in a plant is increased under conditions in which the genetically engineered bacteria are exposed to oxygen. For example, in some embodiments, the genetically engineered bacteria can produce about 1% or more of nitrogen in the plant under conditions in which the genetically engineered bacteria are exposed to oxygen. Such conditions can include, but are not limited to, when the soil into which the plant is planted, or will be planted into, can have at least about 0.5% oxygen. For example, at least about 0.75%, about 1%, about 1.25%, about 1.5%, about 1.75%, about 2%, about 2%, about 2.25%, about 2.5%, about 2.75%, or about 3% or more oxygen. In some embodiments, the at least one modification in a gene regulating nitrogen fixation or assimilation in the genetically engineered bacteria results in an increased level of nitrogenase activity in the presence of at least about 0.5% oxygen than in non-engineered bacteria of the same species. For example, at least about 0.75%, about 1%, about 1.25%, about 1.5%, about 1.75%, about 2%, about 2%, about 2.25%, about 2.5%, about 2.75%, or about 3% oxygen. An exemplary method for measuring nitrogen fixation of bacteria in the presence of oxygen is described in Example 3.

Also provided herein are methods of increasing the biomass of a plant (e.g., overall biomass, root and/or shoot biomass), comprising contacting the plant, a part of the plant, or soil into which the plant is planted or will be planted, with a plurality of any of the genetically engineered bacteria described herein.

In some embodiments, a combination of genetically engineered bacteria can be used. For example, in some embodiments, methods of increasing the amount of atmospheric derived nitrogen in a plant can include contacting the plant, a part of the plant, or soil into which the plant is planted with a plurality of genetically engineered Klebsiella variicola bacteria and a plurality of genetically engineered Kosakonia sacchari bacteria. In some embodiments, the Klebsiella variicola bacterium has a higher nitrogenase activity than the Kosakonia sacchari bacterium. In some embodiments, the Kosakonia sacchari bacterium has a higher growth rate than the Klebsiella variicola bacterium. In some embodiments, the a plurality of genetically engineered Klebsiella variicola bacteria and a plurality of genetically engineered Kosakonia sacchari bacteria are applied to the plant, a part of the plant, or soil into which the plant is planted, or will be planted, simultaneously. In some embodiments, the a plurality of genetically engineered Klebsiella variicola bacteria and a plurality of genetically engineered Kosakonia sacchari bacteria are applied to the plant, a part of the plant, or soil into which the plant is planted or will be planted, sequentially.

In some embodiments, provided herein are methods of increasing colonization in at least two different niches of the rhizosphere of a plant, the method comprising contacting the plant, a part of the plant, or soil into which the plant is planted with a plurality of any of the genetically engineered bacteria described herein. A “niche” as used herein can refers to the ecological space a microbe (e.g., a genetically engineered bacterium) occupies. For example, a niche can describe how a microbe responds to the distribution of resources, physical parameters (e.g., host tissue space) and competitors (e.g., by growing when resources are abundant) and how it in turn alters those same factors (e.g., limiting access to resources by other organisms). In some embodiments, at least two pluralities of genetically engineered bacteria are contacted with the plant, a part of the plant, or soil into which the plant is planted, wherein the first plurality occupies a different niche than the second plurality. In some embodiments, the first plurality is a plurality of genetically engineered Klebsiella variicola bacteria and the second plurality is a plurality of genetically engineered Kosakonia sacchari bacteria.

In some embodiments, genetically engineered bacteria from different niches have one or more of: different nutrient utilization; different temporal occupation; different oxygen adaptability; and different spatial occupation. In some embodiments, the nutrient is carbon. In some embodiments, a strain of bacteria in the rhizosphere of a plant utilize at least one carbon source that is different than the carbon source of a different strain of bacteria in the rhizosphere of the plant. In some embodiments, a strain of bacteria in the rhizosphere of a plant utilize at least one carbon source at a different rate than the carbon source of a different strain of bacteria in the rhizosphere of the plant. In some embodiments, a strain of bacteria in the rhizosphere of a plant is able to fixate nitrogen at a higher rate in the presence of oxygen (e.g., oxygen in the soil the plant is planted in) a different strain of bacteria in the rhizosphere of the plant.

In some embodiments, the bacteria cans produce about 1% or more of nitrogen in the plant (e.g. about 2%, about 5%, about 10%, or more). This can represent a nitrogen-fixation capability of at least 2-fold as compared to the plant in the absence of the bacteria. In some embodiments, the bacteria are capable of fixing atmospheric nitrogen in the presence of exogenous nitrogen In some embodiments, the bacteria can produce the nitrogen in the presence of fertilizer supplemented with glutamine, urea, nitrates or ammonia.

Genetic modifications can be any genetic modification described herein, including examples provided above, in any number and any combination. In some embodiments, the genetic modification introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic modification may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; or decreased uridylyl-transfersae activity of GlnD.

In some embodiments, the at least one modification in the gene regulating nitrogen fixation or assimilation can result in one or more of: constitutive expression of the nifA gene in nitrogen limiting and non-nitrogen limiting conditions, activity of nifA in non-nitrogen limiting conditions, decreased uridylyl-transferase activity of GlnD, decreased adenylyl-removing activity of GlnE, and increased nitrogen excretion.

In some embodiments, genetically engineered bacteria as provided herein can comprise at least one modification in one or more genes regulating nitrogen fixation or assimilation selected from nifL, glnD, glnE, NtrC, and nifA.

The genetic modification introduced into one or more bacteria of the methods disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic modification may be produced by chemical mutagenesis. The plants grown in step (c) may be exposed to biotic or abiotic stressors.

In some embodiments, the genetically engineered bacteria colonize the root surface of the plant. In some embodiments, provided herein are methods of increasing the total space on the root surface of a plant occupied by bacteria that can fix nitrogen in the presence of nitrogen that include contacting the plant, a part of the plant, or soil into which the plant is planted with genetically engineered bacteria as described herein. In some embodiments, the genetically engineered bacteria exhibit colonization levels of at least about 10³ CFU/g root fresh weight (FW). For example, at least about 10⁴ CFU/g root fresh weight (FW), at least about 10⁵ CFU/g root fresh weight (FW), or at least about 10⁶ CFU/g root fresh weight (FW).

In some embodiments, genetically engineered bacteria of the present disclosure produce fixed N of at least about 2×10⁻¹³ mmol of N per CFU per hour, about 2.5×10⁻¹³ mmol of N per CFU per hour, about 3×10⁻¹³ mmol of N per CFU per hour, about 3.5×10⁻¹³ mmol of N per CFU per hour, about 4×10⁻¹³ mmol of N per CFU per hour, about 4.5×10⁻¹³ mmol of N per CFU per hour, about 5×10⁻¹³ mmol of N per CFU per hour, about 5.5×10⁻¹³ mmol of N per CFU per hour, about 6×10⁻¹³ mmol of N per CFU per hour, about 6.5×10⁻¹³ mmol of N per CFU per hour, about 7×10⁻¹³ mmol of N per CFU per hour, about 7.5×10⁻¹³ mmol of N per CFU per hour, about 8×10⁻¹³ mmol of N per CFU per hour, about 8.5×10⁻¹³ mmol of N per CFU per hour, about 9×10⁻¹³ mmol of N per CFU per hour, about 9.5×10⁻¹³ mmol of N per CFU per hour, or about 10×10⁻¹³ mmol of N per CFU per hour.

In some embodiments, genetically engineered bacteria of the present disclosure produce fixed N of at least about 2×10⁻¹² mmol of N per CFU per hour, about 2.25×10⁻¹² mmol of N per CFU per hour, about 2.5×10⁻¹² mmol of N per CFU per hour, about 2.75×10⁻¹² mmol of N per CFU per hour, about 3×10⁻¹² mmol of N per CFU per hour, about 3.25×10⁻¹² mmol of N per CFU per hour, about 3.5×10⁻¹² mmol of N per CFU per hour, about 3.75×10⁻¹² mmol of N per CFU per hour, about 4×10⁻¹² mmol of N per CFU per hour, about 4.25×10⁻¹² mmol of N per CFU per hour, about 4.5×10⁻¹² mmol of N per CFU per hour, about 4.75×10⁻¹² mmol of N per CFU per hour, about 5×10⁻¹² mmol of N per CFU per hour, about 5.25×10⁻¹² mmol of N per CFU per hour, about 5.5×10⁻¹² mmol of N per CFU per hour, about 5.75×10⁻¹² mmol of N per CFU per hour, about 6×10⁻¹² mmol of N per CFU per hour, about 6.25×10⁻¹² mmol of N per CFU per hour, about 6.5×10⁻¹² mmol of N per CFU per hour, about 6.75×10⁻¹² mmol of N per CFU per hour, about 7×10⁻¹² mmol of N per CFU per hour, about 7.25×10⁻¹² mmol of N per CFU per hour, about 7.5×10⁻¹² mmol of N per CFU per hour, about 7.75×10⁻¹² mmol of N per CFU per hour, about 8×10⁻¹² mmol of N per CFU per hour, about 8.25×10⁻¹² mmol of N per CFU per hour, about 8.5×10⁻¹² mmol of N per CFU per hour, about 8.75×10⁻¹² mmol of N per CFU per hour, about 9×10⁻¹² mmol of N per CFU per hour, about 9.25×10⁻¹² mmol of N per CFU per hour, about 9.5×10⁻¹² mmol of N per CFU per hour, about 9.75×10⁻¹² mmol of N per CFU per hour, or about 10×10⁻¹² mmol of N per CFU per hour.

In some embodiments, genetically engineered bacteria of the present disclosure produce fixed N of at least about 5.49×10⁻¹³ mmol of N per CFU per hour. In some embodiments, genetically engineered bacteria of the present disclosure produce fixed N of at least about 4.03×10⁻¹³ mmol of N per CFU per hour. In some embodiments, genetically engineered bacteria of the present disclosure produce fixed N of at least about 2.75×10⁻¹³ mmol of N per CFU per hour.

In some embodiments, genetically engineered bacteria of the present disclosure produce fixed N of at least about 1×10⁻¹⁷ mmol N per bacterial cell per hour. For example, at least about 2×10⁻¹⁷ mmol N per bacterial cell per hour, at least about 2.5×10⁻¹⁷ mmol N per bacterial cell per hour, at least about 3×10⁻¹⁷ mmol N per bacterial cell per hour, at least about 3.5×10⁻¹⁷ mmol N per bacterial cell per hour, at least about 4×10⁻¹⁷ mmol N per bacterial cell per hour, at least about 4.5×10⁻¹⁷ mmol N per bacterial cell per hour, or at least about 5×10⁻¹⁷ mmol N per bacterial cell per hour.

In some embodiments, genetically engineered bacteria of the present disclosure in aggregate produce at least about 15 pounds of fixed N per acre, at least about 20 pounds of fixed N per acre, at least about 25 pounds of fixed N per acre, at least about 30 pounds of fixed N per acre, at least about 35 pounds of fixed N per acre, at least about 40 pounds of fixed N per acre, at least about 45 pounds of fixed N per acre, at least about 50 pounds of fixed N per acre, at least about 55 pounds of fixed N per acre, at least about 60 pounds of fixed N per acre, at least about 65 pounds of fixed N per acre, at least about 70 pounds of fixed N per acre, at least about 75 pounds of fixed N per acre, at least about 80 pounds of fixed N per acre, at least about 85 pounds of fixed N per acre, at least about 90 pounds of fixed N per acre, at least about 95 pounds of fixed N per acre, or at least about 100 pounds of fixed N per acre.

In some embodiments, genetically engineered bacteria of the present disclosure produce fixed N in the amounts disclosed herein over the course of at least about day 0 to about 80 days, at least about day 0 to about 70 days, at least about day 0 to about 60 days, at least about 1 day to about 80 days, at least about 1 day to about 70 days, at least about 1 day to about 60 days, at least about 2 days to about 80 days, at least about 2 days to about 70 days, at least about 2 days to about 60 days, at least about 3 days to about 80 days, at least about 3 days to about 70 days, at least about 3 days to about 60 days, at least about 4 days to about 80 days, at least about 4 days to about 70 days, at least about 4 days to about 60 days, at least about 5 days to about 80 days, at least about 5 days to about 70 days, at least about 5 days to about 60 days, at least about 6 days to about 80 days, at least about 6 days to about 70 days, at least about 6 days to about 60 days, at least about 7 days to about 80 days, at least about 7 days to about 70 days, at least about 7 days to about 60 days, at least about 8 days to about 80 days, at least about 8 days to about 70 days, at least about 8 days to about 60 days, at least about 9 days to about 80 days, at least about 9 days to about 70 days, at least about 9 days to about 60 days, at least about 10 days to about 80 days, at least about 10 days to about 70 days, at least about 10 days to about 60 days, at least about 15 days to about 80 days, at least about 15 days to about 70 days, at least about 15 days to about 60 days, at least about 20 days to about 80 days, at least about 20 days to about 70 days, or at least about 20 days to about 60 days.

In some embodiments, genetically engineered bacteria of the present disclosure produce fixed N in any of the amounts disclosed herein over the course of at least about 80 days±5 days, at least about 80 days±10 days, at least about 80 days±15 days, at least about 80 days±20 days, at least about 75 days±5 days, at least about 75 days±10 days, at least about 75 days±15 days, at least about 75 days±20 days, at least about 70 days±5 days, at least about 70 days±10 days, at least about 70 days±15 days, at least about 70 days±20 days, at least about 60 days±5 days, at least about 60 days±10 days, at least about 60 days±15 days, at least about 60 days±20 days.

In some embodiments, genetically engineered bacteria of the present disclosure produce fixed N in any of the amounts disclosed herein over the course of at least about 10 days to about 80 days, at least about 10 days to about 70 days, or at least about 10 days to about 60 days.

The amount of nitrogen fixation that occurs in the plants described herein may be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

The wild-type nitrogen fixation regulatory cascade can be represented as a digital logic circuit where the inputs O₂ and NH₄⁺ pass through a NOR gate, the output of which enters an AND gate in addition to ATP. In some embodiments, the methods disclosed herein disrupt the influence of NH₄⁺ on this circuit, at multiple points in the regulatory cascade, so that microbes can produce nitrogen even in fertilized fields. However, the methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

Systems for plant growth and measurement of nitrogen incorporation can include a chamber, a gas delivery apparatus, a nutrient delivery apparatus. The system includes a chamber with walls that enclose a spatial volume internal to chamber. System also includes a gas delivery apparatus and a nutrient delivery apparatus connected to a controller via control lines. System can optionally include a sampling apparatus.

Chamber can include any number of walls suitable for enclosing spatial volume, and the wall(a)s can define any shape for chamber. In some embodiments, for example, the wall(s) define a cubic or rectangular prismatic shape for chamber. In certain embodiments, the wall(s) define a spherical or elliptical shape for chamber. More generally, the wall(s) can define any regular or irregular shape for chamber.

At least one surface of at least one wall typically supports one or more plants within the enclosed spatial volume. The height h of chamber is the minimum distance between the plant-supporting surface and a wall surface opposite the plant supporting surface. Upward plant growth generally occurs in a direction parallel to height h, and so the height can be selected to accommodate such growth for one or more different plant types. In some embodiments, h can be 0.5 m or more (e.g., 0.6 m or more, 0.7 m or more, 0.8 m or more, 0.9 m or more, 1.0 m or more, 1.5 m or more, 2.0 m or more, 2.5 m or more, 3.0 m or more, 3.5 m or more, 4.0 m or more, 4.5 m or more, 5.0 m or more, 5.5 m or more, 6.0 m or more, 6.5 m or more, 7.0 m or more, 7.5 m or more, 8.0 m or more, 8.5 m or more, 9.0 m or more, 9.5 m or more, 10.0 m or more, or even more).

In certain embodiments, the height h is sufficiently large so that the entire plant is positioned within the enclosed spatial volume. This provides an important advantage relative to measurement systems in which just the plant roots are enclosed. By placing the entire plant within the enclosed spatial volume, direct assessment of the fixation of nitrogen surrounding the entire plant—as is typical under field growing conditions—and subsequent incorporation of reduced nitrogen by plant tissues can be performed.

In general, the enclosed spatial volume of chamber can be selected as desired to accommodate one or more plants and gases delivered to the plants. In some embodiments, for example, the enclosed spatial volume can be 100 L or more (e.g., 200 L or more, 300 L or more, 400 L or more, 500 L or more, 600 L or more, 700 L or more, 800 L or more, 900 L or more, 1000 L or more, 1500 L or more, 2000 L or more, 2500 L or more, 3000 L or more, 4000 L or more, 5000 L or more, 7000 L or more, 10,000 L or more, 15,000 L or more, 20,000 L or more, 30,000 L or more, 50,000 L or more, or even more).

In some embodiments, chamber is relatively airtight, such that a leakage rate of gases from chamber is relatively small. For example, when chamber is filled with a gas such as nitrogen at a pressure of 1.5 atmospheres (e.g., 152 kPa), a leakage rate of the gas from the chamber can be less than 0.5 L/day (e.g., less than 0.3 L/day, less than 0.1 L/day, less than 0.05 L/day, less than 0.01 L/day, less than 0.005 L/day, less than 0.001 L/day). More generally, when chamber is filled with a gas such as nitrogen at a pressure p at a first time, the gas pressure within the chamber at a second time at least 7 days after the first time can be 0.70p or more (e.g., 0.80p or more, 0.85p or more, 0.90p or more, 0.95p or more, 0.98p or more, 0.99p or more, 0.999p or more, 0.9999p or more, or even more).

The walls of chamber can generally be formed from a variety of materials including, but not limited to, various plastics and metals. Mating walls can be joined by bonding, welding, clamping, and other processes to form wall joints. A variety of structural supporting members can be used to reinforce the walls of chamber, and such members can be formed of the same or different materials than the walls.

During operation of system, controller activates the gas delivery apparatus to deliver one or more gases into the enclosed spatial volume of chamber. Gas delivery apparatus can be implemented in different ways. In some embodiments, gas delivery apparatus is positioned within chamber. Alternatively, in certain embodiments, gas delivery apparatus (or a portion thereof) is positioned external to chamber. Gas delivery apparatus can include one or more gas sources, one or more conduits, and one or more valves. Each of the valves can optionally be connected to controller, which activates the valve(s) to regulate gas delivery from the gas delivery apparatus.

### I. Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizopheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring 15N gas/fertilizer (dilution) with IRMS or NanoSIMS as described herein NanoSIMS is high-resolution secondary ion mass spectrometry. The NanoSIMS technique is a way to investigate chemical activity from biological samples. The catalysis of reduction of oxidation reactions that drive the metabolism of microorganisms can be investigated at the cellular, subcellular, molecular and elemental level. NanoSIMS can provide high spatial resolution of greater than 0.1 μm. NanoSIMS can detect the use of isotope tracers such as ¹³C, ¹⁵N, and ¹⁸O. Therefore, NanoSIMS can be used to the chemical activity nitrogen in the cell.

Automated greenhouses can be used for planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer. Primers designed for the nifH gene can be used to identity diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

Enriching for Microbes with Nitrogen Fixation Capabilities Using Bioinformatics

Bioinformatic tools can be used to identify and isolate plant growth promoting rhizobacteria, which are selected based on their ability to perform nitrogen fixation. Microbes with high nitrogen fixing ability can promote favorable traits in plants. Bioinformatic modes of analysis for the identification of rhizobacteria include, but are not limited to, genomics, metagenomics, targeted isolation, gene sequencing, transcriptome sequencing, and modeling.

Genomics analysis can be used to identify rhizobacteria and confirm the presence of mutations with methods of Next Generation Sequencing as described herein and microbe version control.

Metagenomics can be used to identify and isolate rhizobacteria using a prediction algorithm for colonization. Metadata can also be used to identify the presence of an engineered strain in environmental and greenhouse samples.

Transcriptomic sequencing can be used to predict genotypes leading to rhizobacteria phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector may consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Non-Transgenic Engineering of Microbes

A microbial population with favorable traits can be obtained via directed evolution. Direct evolution is an approach wherein the process of natural selection is mimicked to evolve proteins or nucleic acids towards a user-defined goal. An example of direct evolution is when random mutations are introduced into a microbial population, the microbes with the most favorable traits are selected, and the growth of the selected microbes is continued. The most favorable traits in rhizobacteria can be in nitrogen fixation. The method of directed evolution may be iterative and adaptive based on the selection process after each iteration.

Rhizobacteria with high capability of nitrogen fixation can be generated. The evolution of rhizobacteria can be carried out via the introduction of genetic modification. Genetic modification can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved rhizobacteria properties, improved colonization of cereals, increased oxygen sensitivity, increased nitrogen fixation, and increased ammonia excretion. Intrageneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Selection of Plant Growth Promoting Microbes Based on Nitrogen Fixation

The microbial colonies can be screened using various assays to assess nitrogen fixation. One way to measure nitrogen fixation is via a single fermentative assay, which measures nitrogen excretion. An alternative method is the acetylene reduction assay (ARA) with in-line sampling over time. ARA can be performed in high throughput plates of microtube arrays. ARA can be performed with live plants and plant tissues. The media formulation and media oxygen concentration can be varied in ARA assays. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria may be cultured in the presence or absence of a nitrogen source. For example, the bacteria may be cultured with glutamine, ammonia, urea or nitrates.

### Guided Microbial Remodeling—An Overview

Guided microbial remodeling is a method to systematically identify and improve the role of species within the crop microbiome. In some embodiments, and according to a particular methodology of grouping/categorization, the method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters within a microbe's genome, and 3) screening and selection of new microbial genotypes that produce desired crop phenotypes.

To systematically assess the improvement of strains, a model is created that links colonization dynamics of the microbial community to genetic activity by key species. The model is used to predict genetic targets for non-intergeneric genetic remodeling (i.e. engineering the genetic architecture of the microbe in a non-transgenic fashion). Rational improvement of the crop microbiome can be used to increase soil biodiversity, tune impact of keystone species, and/or alter timing and expression of important metabolic pathways.

The aforementioned “Guided Microbial Remodeling” process is further elaborated upon in International Publication Nos. WO 2020/006246 and WO 2020/014498, each of which are incorporated by reference herein in their entireties. The genetically engineered bacteria of the present disclosure can be generating using a suicide plasmid, e.g., as described in International Publication No. WO 2020/00624.

### Serial Passage

Production of bacteria to improve plant traits (e.g., nitrogen fixation) can be achieved through serial passage. The production of this bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic modification into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as 10 to 20 plants, or 20 or more, 50 or more, 100 or more, 300 or more, 500 or more, or 1000 or more plants.

In addition to obtaining a plant with an improved trait, a bacterial population comprising bacteria comprising one or more genetic modifications introduced into one or more genes (e.g., genes regulating nitrogen fixation) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis may occur of isolated bacteria in step (a). Phenotypic and/or genotypic information may be obtained using techniques including: high through-put screening of chemical components of plant origin, sequencing techniques including high throughput sequencing of genetic material, differential display techniques (including DDRT-PCR, and DD-PCR), nucleic acid microarray techniques, RNA-sequencing (Whole Transcriptome Shotgun Sequencing), and qRT-PCR (quantitative real time PCR). Information gained can be used to obtain community profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nifD gene. Example processes of taxonomic profiling to determine taxa present in a population are described in US20140155283. Bacterial identification may comprise characterizing activity of one or more genes or one or more signaling pathways, such as genes associated with the nitrogen fixation pathway. Synergistic interactions (where two components, by virtue of their combination, increase a desired effect by more than an additive amount) between different bacterial species may also be present in the bacterial populations.

### Genetic Modification—Locations and Sources of Genomic Alteration

The genetic modification may be a gene selected from the group consisting of: nifA, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, niM, nifF, nifB, and nifQ. The genetic modification may be a modification in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, and NAD+-dinitrogen-reductase aDP-D-ribosyltransferase. The genetic modification may be a mutation that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-transferase activity of GlnD. The genetic modification can be a modification in a gene selected from the group consisting of: bcsii, bcsiii, yjbE, jhaB, pehA, otsB, treZ, glsA2, and combinations thereof. In some embodiments, a genetic modification can be a modification in any of the genes described throughout this disclosure.

Introducing a genetic modification may comprise insertion and/or deletion of one or more nucleotides at a target site, such as 1, 2, 3, 4, 5, 10, 25, 50, 100, 250, 500, or more nucleotides. The genetic modification introduced into one or more bacteria of the methods disclosed herein may be a knock-out mutation (e.g. deletion of a promoter, insertion or deletion to produce a premature stop codon, deletion of an entire gene), or it may be elimination or abolishment of activity of a protein domain (e.g. point mutation affecting an active site, or deletion of a portion of a gene encoding the relevant portion of the protein product), or it may alter or abolish a regulatory sequence of a target gene. One or more regulatory sequences may also be inserted, including heterologous regulatory sequences and regulatory sequences found within a genome of a bacterial species or genus corresponding to the bacteria into which the genetic modification is introduced. Moreover, regulatory sequences may be selected based on the expression level of a gene in a bacterial culture or within a plant tissue. The genetic modification may be a pre-determined genetic modification that is specifically introduced to a target site. The genetic modification may be a random mutation within the target site. The genetic modification may be an insertion or deletion of one or more nucleotides. In some cases, a plurality of different genetic modifications (e.g. 2, 3, 4, 5, 10, or more) are introduced into one or more of the isolated bacteria before exposing the bacteria to plants for assessing trait improvement. The plurality of genetic modifications can be any of the above types, the same or different types, and in any combination. In some cases, a plurality of different genetic modifications are introduced serially, introducing a first genetic modification after a first isolation step, a second genetic modification after a second isolation step, and so forth so as to accumulate a plurality of genetic modifications in bacteria imparting progressively improved traits on the associated plants.

### Genetic Modification—Methods of Introducing Genomic Alteration

Genetic modifications can have any number of effects, such as the increase or decrease of some biological activity, including gene expression, metabolism, and cell signaling. Genetic modifications can be specifically introduced to a target site, or introduced randomly. A variety of molecular tools and methods are available for introducing genetic modification. For example, genetic modification can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic modification include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, γ-irradiation, X-rays, and fast neutron bombardment. Genetic modification can also be introduced into a nucleic acid using, e.g., trimethylpsoralen with ultraviolet light. Random or targeted insertion of a mobile DNA element, e.g., a transposable element, is another suitable method for generating genetic modification. Genetic modifications can be introduced into a nucleic acid during amplification in a cell-free in vitro system, e.g., using a polymerase chain reaction (PCR) technique such as error-prone PCR. Genetic modifications can be introduced into a nucleic acid in vitro using DNA shuffling techniques (e.g., exon shuffling, domain swapping, and the like). Genetic modifications can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic modifications are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic modifications introduced into microbes may be classified as transgenic, cisgenic, intragenomic, intrageneric, intergeneric, synthetic, evolved, rearranged, or SNPs.

Genetic modification may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, Nitrogen uptake, glutamine biosynthesis, colonization pathways, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic modification can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA, by which the mutagenized unmethylated chains are recovered; or 4) circularization of the mutated PCR products in an additional ligation reaction to increase the transformation efficiency of mutated DNA. Further description of exemplary methods can be found in e.g. U.S. Pat. Nos. 7,132,265, 6,713,285, 6,673,610, 6,391,548, 5,789,166, 5,780,270, 5,354,670, 5,071,743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic modifications can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100,000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers—DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and US20140301990.

Mutagens that create primarily point mutations and short deletions, insertions, transversions, and/or transitions, including chemical mutagens or radiation, may be used to create genetic modifications. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Introducing genetic modification may be an incomplete process, such that some bacteria in a treated population of bacteria carry a desired mutation while others do not. In some cases, it is desirable to apply a selection pressure so as to enrich for bacteria carrying a desired genetic modification. Traditionally, selection for successful genetic variants involved selection for or against some functionality imparted or abolished by the genetic modification, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic modification need be introduced (e.g. without also requiring a selectable marker). In this case, the selection pressure can comprise cleaving genomes lacking the genetic modification introduced to a target site, such that selection is effectively directed against the reference sequence into which the genetic modification is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), and a meganuclease. Such a process is similar to processes for enhancing homologous recombination at a target site, except that no template for homologous recombination is provided. As a result, bacteria lacking the desired genetic modification are more likely to undergo cleavage that, left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic modification, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family (SEQ ID NO: 12), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

### Genetic Modification—Methods of Identification

The microbes of the present disclosure can be identified by one or more genetic modifications or alterations, which have been introduced into the microbe. One method by which a genetic modification or alteration can be identified is via reference to a SEQ ID NO that contains a portion of the microbe's genomic sequence that is sufficient to identify the genetic modification or alteration.

Further, in the case of microbes that have not had a genetic modification or alteration (e.g. a wild type, WT) introduced into their genomes, the disclosure can utilize 16S nucleic acid sequences to identify said microbes. A 16S nucleic acid sequence is an example of a “molecular marker” or “genetic marker,” which refers to an indicator that can be used in methods for visualizing differences in species of bacteria. Examples of other such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further can include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions when compared against one another. Furthermore, the disclosure utilizes unique sequences found in genes of interest (e.g. nifH, nifD, nifK, nifL, nifA, glnE, amtB, etc.) to identify microbes disclosed herein.

The primary structure of the major rRNA subunit 16S comprises a particular combination of conserved, variable, and hypervariable regions that evolve at different rates and can enable the resolution of both very ancient lineages such as domains, and more modern lineages such as genera. The secondary structure of the 16S subunit includes approximately 50 helices that result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

### Genetic Modification—Methods of Detection: Primers, Probes, and Assays

The present disclosure provides primers, probes, and assays that are useful for detecting the microbes taught herein. In some embodiments, the disclosure provides for methods of detecting the WT parental strains. In some embodiments, the disclosure provides for methods of detecting the non-intergeneric engineered microbes derived from the WT strains. In some embodiments, the present disclosure provides methods of identifying non-intergeneric genetic alterations in a microbe.

In some embodiments, the genomic engineering methods of the present disclosure lead to the creation of non-natural nucleotide “junction” sequences in the derived non-intergeneric microbes. These non-naturally occurring nucleotide junctions can be used as a type of diagnostic that is indicative of the presence of a particular genetic alteration in a microbe taught herein.

The present techniques are able to detect these non-naturally occurring nucleotide junctions via the utilization of specialized quantitative PCR methods, including uniquely designed primers and probes. In some embodiments, the probes of the disclosure bind to the non-naturally occurring nucleotide junction sequences. In some embodiments, traditional PCR is utilized. In some embodiments, real-time PCR is utilized. In some embodiments, quantitative PCR (qPCR) is utilized.

Thus, the disclosure can cover the utilization of two common methods for the detection of PCR products in real-time: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence. In some embodiments, only the nonnaturally occurring nucleotide junction will be amplified via the taught primers, and consequently can be detected either via a non-specific dye, or via the utilization of a specific hybridization probe. In some embodiments, the primers of the disclosure are chosen such that the primers flank either side of a junction sequence, such that if an amplification reaction occurs, then said junction sequence is present.

Some embodiments of the disclosure involve non-naturally occurring nucleotide junction sequence molecules per se, along with other nucleotide molecules that are capable of binding to said non-naturally occurring nucleotide junction sequences under mild to stringent hybridization conditions. In some embodiments, the nucleotide molecules that are capable of binding to said non-naturally occurring nucleotide junction sequences under mild to stringent hybridization conditions are termed “nucleotide probes.”

In some embodiments, genomic DNA can be extracted from samples and used to quantify the presence of microbes of the disclosure by using qPCR. The primers utilized in the qPCR reaction can be primers designed by Primer Blast (www.ncbi.nlm.nih.gov/tools/primer-blast/) to amplify unique regions of the wild-type genome or unique regions of the engineered non intergeneric mutant strains. The qPCR reaction can be carried out using the SYBR GreenER qPCR SuperMix Universal (Thermo Fisher PIN 11762100) kit, using only forward and reverse amplification primers; alternatively, the Kapa Probe Force kit (Kapa Biosystems PIN KK4301) can be used with amplification primers and a TaqMan probe containing a FAM dye label at the 5′ end, an internal ZEN quencher, and a minor groove binder and fluorescent quencher at the 3′ end (Integrated DNA Technologies).

qPCR reaction efficiency can be measured using a standard curve generated from a known quantity of gDNA from the target genome. Data can be normalized to genome copies per g fresh weight using the tissue weight and extraction volume.

Quantitative polymerase chain reaction (qPCR) is a method of quantifying, in real time, the amplification of one or more nucleic acid sequences. The real time quantification of the PCR assay permits determination of the quantity of nucleic acids being generated by the PCR amplification steps by comparing the amplifying nucleic acids of interest and an appropriate control nucleic acid sequence, which can act as a calibration standard.

TaqMan probes are often utilized in qPCR assays that require an increased specificity for quantifying target nucleic acid sequences. TaqMan probes comprise an oligonucleotide probe with a fluorophore attached to the 5′ end and a quencher attached to the 3′ end of the probe. When the TaqMan probes remain as is with the 5′ and 3′ ends of the probe in close contact with each other, the quencher prevents fluorescent signal transmission from the fluorophore. TaqMan probes are designed to anneal within a nucleic acid region amplified by a specific set of primers. As the Taq polymerase extends the primer and synthesizes the nascent strand, the 5′ to 3′ exonuclease activity of the Taq polymerase degrades the probe that annealed to the template. This probe degradation releases the fluorophore, thus breaking the close proximity to the quencher and allowing fluorescence of the fluorophore. Fluorescence detected in the qPCR assay is directly proportional to the fluorophore released and the amount of DNA template present in the reaction.

The features of qPCR can allow the practitioner to eliminate the labor-intensive post-amplification step of gel electrophoresis preparation, which is generally required for observation of the amplified products of traditional PCR assays. The benefits of qPCR over conventional PCR can be considerable, and include increased speed, ease of use, reproducibility, and quantitative ability.

### Improvement of Traits

Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

In some embodiments, a trait to be introduced or improved is nitrogen fixation, as described herein. In some embodiments, a trait to be introduced or improved is colonization potential, as described herein. In some embodiments, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions of the present disclosure may be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant's nitrogen, which may represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from the first plant before introducing any genetic modification. In some cases, the bacteria produce 5% or more of a plant's nitrogen. The desired level of nitrogen fixation may be achieved after repeating the steps of introducing genetic modification, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

### Measuring Nitrogen Delivered in an Agriculturally Relevant Field Context

In the field, the amount of nitrogen delivered can be determined by the function of colonization multiplied by the activity.

$$Nitrogen delivered = {f}_{{Time \& Space}^{*}} Colonization \times Activity$$

The above equation requires (1) the average colonization per unit of plant tissue, and (2) the activity as either the amount of nitrogen fixed or the amount of ammonia excreted by each microbial cell. To convert to pounds of nitrogen per acre, corn growth physiology is tracked over time, e.g., size of the plant and associated root system throughout the maturity stages.

The pounds of nitrogen delivered to a crop per acre-season can be calculated by the following equation:

$$Nitrogen delivered = Plant Tissue ( t ) \times Colonization \times Activity ( t ) dt$$

The Plant Tissue(t) is the fresh weight of corn plant tissue over the growing time (t). Values for reasonably making the calculation are described in detail in the publication entitled Roots, Growth and Nutrient Uptake (Mengel. Dept. of Agronomy Pub. #AGRY-95-08 (Rev. May-95. p. 1-8.).

The Colonization (t) is the amount of the microbes of interest found within the plant tissue, per gram fresh weight of plant tissue, at any particular time, t, during the growing season. In the instance of only a single timepoint available, the single timepoint is normalized as the peak colonization rate over the season, and the colonization rate of the remaining timepoints are adjusted accordingly.

Activity(t) is the rate at which N is fixed by the microbes of interest per unit time, at any particular time, t, during the growing season. In the embodiments disclosed herein, this activity rate is approximated by in vitro ARA in ARA media in the presence of 5 mM glutamine or ammonium excretion assay in ARA media in the presence of 5 mM ammonium ions.

The Nitrogen delivered amount is then calculated by numerically integrating the above function. In cases where the values of the variables described above are discretely measured at set timepoints, the values in between those timepoints are approximated by performing linear interpolation.

### Bacterial Species

Microbes useful in the methods and compositions disclosed herein may be obtained from any source. In some cases, microbes may be bacteria, archaea, protozoa or fungi. The microbes of this disclosure may be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein may be spore forming microbes, for example spore forming bacteria. In some cases, bacteria useful in the methods and compositions disclosed herein may be Gram positive bacteria or Gram negative bacteria. In some cases, the bacteria may be an endospore forming bacteria of the Firmicute phylum. In some cases, the bacteria may be a diazatroph. In some cases, the bacteria may not be a diazotroph. In some cases, the microbe is a biocontrol microbe. Biocontrol microbes, or microbial biocontrol agents, control the grown and/or population size of a target species through biological interactions (e.g. competition for resources, causing disease in the target species, production of allelochemicals or toxins, or influencing crop plants).

The methods and compositions of this disclosure may be used with an archaea, such as, for example, Methanothermobacter thermoautotrophicus.

In some cases, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus lichenformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus unflagellatus, Bradyrhizobium japonicum, Brevibacillus brevis Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some cases the bacterium may be Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum or Rhizobium etli.

In some cases the bacterium may be a species of Clostridium, for example Clostridium pasteurianum, Clostridium beijerinckii, Clostridium perfringens, Clostridium tetani, Clostridium acetobutylicum.

In some cases, bacteria used with the methods and compositions of the present disclosure may be cyanobacteria. Examples of cyanobacterial genuses include Anabaena (for example Anagaena sp. PCC7120), Nostoc (for example Nostoc punctiforme), or Synechocystis (for example Synechocystis sp. PCC6803).

In some cases, bacteria used with the methods and compositions of the present disclosure may belong to the phylum Chlorobi, for example Chlorobium tepidum.

In some cases, microbes used with the methods and compositions of the present disclosure may comprise a gene homologous to a known NifH gene. Sequences of known NifH genes may be found in, for example, the Zehr lab NifH database, (wwwzehr.pmc.ucsc.edu/nifH_Database_Public/, Apr. 4, 2014), or the Buckley lab NifH database (www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau00l.). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Zehr lab NifH database, (www.zehr.pmc.ucsc.edu/nifH_Database_Public/, Apr. 4, 2014). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb, tuber, root, and rhizosomes. In some cases, bacteria are isolated from a seed. The parameters for processing samples may be varied to isolate different types of associative microbes, such as rhizospheric, epiphytes, or endophytes. Bacteria may also be sourced from a repository, such as environmental strain collections, instead of initially isolating from a first plant. The microbes can be genotyped and phenotyped, via sequencing the genomes of isolated microbes; profiling the composition of communities inplanta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein may apply to bacteria able to self-propagate efficiently on the leaf surface, root surface, or inside plant tissues without inducing a damaging plant defense reaction, or bacteria that are resistant to plant defense responses. The bacteria described herein may be isolated by culturing a plant tissue extract or leaf surface wash in a medium with no added nitrogen. However, the bacteria may be unculturable, that is, not known to be culturable or difficult to culture using standard methods known in the art. The bacteria described herein may be an endophyte or an epiphyte or a bacterium inhabiting the plant rhizosphere (rhizospheric bacteria). The bacteria obtained after repeating the steps of introducing genetic modification, exposure to a plurality of plants, and isolating bacteria from plants with an improved trait one or more times (e.g. 1, 2, 3, 4, 5, 10, 15, 25, or more times) may be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-borne bacterial endophyte is capable of surviving desiccation.

The bacterial isolated according to methods of the disclosure, or used in methods or compositions of the disclosure, can comprise a plurality of different bacterial taxa in combination. By way of example, the bacteria may include Proteobacteria (such as Pseudomonas, Enterobacter, Stenotrophomonas, Burkholderia, Paraburkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetabacterium), and Actinobacteria (such as Streptomyces, Rhodacoccus, Microbacterium, and Curtobacterium). The bacteria used in methods and compositions of this disclosure may include nitrogen fixing bacterial consortia of two or more species. In some cases, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture. Examples of bacterial genuses which may be found in a nitrogen fixing bacterial consortia include, but are not limited to, Herbaspirillum, Azospirillum, Enterobacter, and Bacillus.

Bacteria that can be produced by the methods disclosed herein include Azotobacter sp., Bradyrhizobium sp., Klebsiella sp., and Sinorhizobium sp. In some cases, the bacteria may be selected from the group consisting of: Azotobacter vinelandii, Bradyrhizobium japonicum, Klebsiella pneumoniae, and Sinorhizobium meliloti. In some cases, the bacteria may be of the genus Enterobacter or Rahnella. In some cases, the bacteria may be of the genus Frankia, or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some cases, the bacteria may be of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria isolated according to methods of the disclosure can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, 25 Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera Incertae sedis, Xanthomonas, and Zimmermannella.

In some embodiments, a bacterial species selected from at least one of the following genera is utilized: Enterobacter, Klebsiella, Kosakonia, and Rahnella. In some cases, a combination of bacterial species from the following genera are utilized: Enterobacter, Klebsiella, Kosakonia, and Rahnella. In some cases, the species utilized can be one or more of: Enterobacter sacchari, Klebsiella variicola, Kosakonia sacchari, and Rahnella aquatilis.

In some cases, a Gram positive microbe can have a Molybdenum-Iron nitrogenase system comprising: nifH, nifD, nifK, nifB, nifE, nifN, nifX, hesA, nifV, nifW, nifU, nifS, nifil, and nifl2. In some cases, a Gram positive microbe can have a vanadium nitrogenase system comprising: vnjDG, vnfK, vnjE, vnjN, vupC, vupB, vupA, vnjV, vnjRI, vnjH, vnjR2, vnifA (transcriptional regulator). In some cases, a Gram positive microbe can have an iron-only nitrogenase system comprising: anfK, anfG, anjD, anjH, anifA (transcriptional regulator). In some cases, a Gram positive microbe can have a nitrogenase system comprising glnB, and glnK (nitrogen signaling proteins). Some examples of enzymes involved in nitrogen metabolism in Gram positive microbes include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate-ammonia ligase/asparagine synthetase), and ansA/ansZ (asparaginase). Some examples of proteins involved in nitrogen transport in Gram positive microbes include amtB (ammonium transporter), glnK (regulator of ammonium transport), glnPHQ/glnQHMP (ATP-dependent glutamine/glutamate transporters), glnT/alsT/yrbD/yjlA (glutamine-like proton symport transporters), and gltP/gltT/yhcllnqt (glutamate-like proton symport transporters).

Examples of Gram positive microbes that can be of particular interest include Paenibacillus polymixa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Mycobacterium jlaum, Mycobacterium sp., Arthrobacter sp., Agromyces sp., Corynebacterium autitrophicum, Corynebacterium sp., Micromonspora sp., Propionibacteria sp., Streptomyces sp., and Microbacterium sp.

Some examples of genetic alterations that can be made in Gram positive microbes include: deleting glnR to remove negative regulation of BNF in the presence of environmental nitrogen, inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback inhibited (FBI-GS) state, to reduce ammonium assimilation by the GS-GOGAT pathway.

In some cases, glnR is the main regulator of N metabolism and fixation in Paenibacillus species. In some cases, the genome of a Paenibacillus species does not contain a gene to produce glnR. In some cases, the genome of a Paenibacillus species does not contain a gene to produce glnE or glnD. In some cases, the genome of a Paenibacillus species does not contain a gene to produce glnB or glnK. For example, Paenibacillus sp. WLY78 doesn't contain a gene for glnB, or its homologs found in the archaeon Methanococcus maripaludis, nifil and nifl2. In some cases, the genomes of Paenibacillus species can be variable. For example, Paenibacillus polymixa E68 1 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnPHQ controlled by GlnR. Paenibacillus riograndensis SBR5 contains a standard glnRA operon, anfdx gene, a main nif operon, a secondary nif operon, and an anf operon (encoding iron-only nitrogenase). Putative glnR/tnrA sites were found upstream of each of these operons. GlnR does regulate all of the above operons, except the anf operon. GlnR can bind to each of these regulatory sequences as a dimer.

Paenibacillus N-fixing strains can fall into two subgroups: Subgroup I, which contains only a minimal nif gene cluster and subgroup II, which contains a minimal cluster, plus an uncharacterized gene between nifX and hesA, and often other clusters duplicating some of the nif genes, such as nifH, nifHDK, nifBEN, or clusters encoding vanadaium nitrogenase (vnj) or iron-only nitrogenase (anj) genes.

In some cases, the genome of a Paenibacillus species does not contain a gene to produce glnB or glnK. In some cases, the genome of a Paenibacillus species contains a minimal nif cluster with 9 genes transcribed from a sigma-70 promoter. In some cases, a Paenibacillus nif cluster can be negatively regulated by nitrogen or oxygen. In some cases, the genome of a Paenibacillus species does not contain a gene to produce sigma-54. For example, Paenibacillus sp. WLY78 does not contain a gene for sigma-54. In some cases, a nif cluster can be regulated by glnR, and/or TnrA. In some cases, activity of a nif cluster can be altered by altering activity of glnR, and/or TnrA.

In Bacilli, glutamine synthetase (GS) is feedback-inhibited by high concentrations of intracellular glutamine, causing a shift in confirmation (referred to as FBI GS). Nif clusters contain distinct binding sites for the regulators GlnR and TnrA in several Bacilli species. GlnR binds and represses gene expression in the presence of excess intracellular glutamine and AMP. A role of GlnR can be to prevent the influx and intracellular production of glutamine and ammonium under conditions of high nitrogen availability. TnrA can bind and/or activate (or repress) gene expression in the presence of limiting intracellular glutamine, and/or in the presence of FBI-GS. In some cases, the activity of a Bacilli nif cluster can be altered by altering the activity of GlnR.

Feedback-inhibited glutamine synthetase (FBI-GS) can bind GlnR and stabilize binding of GlnR to recognition sequences. Several bacterial species have a GlnR/TnrA binding site upstream of the nif cluster. Altering the binding of FBI-GS and GlnR can alter the activity of the nif pathway.

### Sources of Microbes

The bacteria (or any microbe according to the disclosure) can be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria (or any microbe according to the disclosure) are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The bacteria may be isolated from plant tissue. This isolation can occur from any appropriate tissue in the plant, including for example root, stem and leaves, and plant reproductive tissues. By way of example, conventional methods for isolation from plants typically include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

Biologically pure cultures of Rahnella aquatilis and Enterobacter sacchari were deposited on Jul. 14, 2015 with the American Type Culture Collection (ATCC; an International Depositary Authority), Manassas, VA, USA, and assigned ATTC Patent Deposit Designation numbers PTA-122293 and PTA-122294, respectively. These deposits were made under the provisions of the Budapest Treaty on the International Recognition of the Deposit of Microorganisms for the Purpose of Patent Procedure and the Regulations (Budapest Treaty).

Enterobacter sacchari has now been reclassified as Kosakonia sacchari, the name for the organism may be used interchangeably herein.

Many microbes of the present disclosure are derived from two wild-type strains. Strain C1006 is a bacterial species previously classified in the genus Enterobacter (see aforementioned reclassification into Kosakonia). Strain C1019 is a bacterial species classified in the genus Rahnella. It is noted that strains comprising CM in the name are mutants of the strains depicted immediately to the left of said CM strain. The deposit information forthe C1006 Kosakonia wild type (WT) and CI019 Rahnella WT are found in the below Table 1.

Some microorganisms described in this application were deposited on Jan. 6, 2017 or Aug. 11, 2017 with the Bigelow National Center for Marine Algae and Microbiota (NCMA), located at 60 Bigelow Drive, East Boothbay, Maine 04544, USA As aforementioned, all deposits were made under the terms of the Budapest Treaty on the International Recognition of the Deposit of Microorganisms for the Purposes of Patent Procedure. The Bigelow National Center for Marine Algae and Microbiota accession numbers and dates of deposit for the aforementioned Budapest Treaty deposits are provided in Table 1.

Biologically pure cultures of Kosakonia sacchari (WT), Rahnella aquatilis (WT), and a variant/remodeled Kosakonia sacchari strain were deposited on Jan. 6, 2017 with the NCMA, located at 60 Bigelow Drive, East Boothbay, Maine 04544, USA, and assigned NCMA Patent Deposit Designation numbers 201701001, 201701003, and 201701002, respectively. The applicable deposit information is found below in Table 1.

Biologically pure cultures of variant/remodeled Kosakonia sacchari strains were deposited on Aug. 11, 2017 with the NCMA, located at 60 Bigelow Drive, East Boothbay, Maine 04544, USA, and assigned NCMA Patent Deposit Designation numbers 201708004, 201708003, and 201708002, respectively. The applicable deposit information is found below in Table 1.

A biologically pure culture of Klebsiella variicola (WT) was deposited on Aug. 11, 2017 with the NCMA, located at 60 Bigelow Drive, East Boothbay, Maine 04544, USA, and assigned NCMA Patent Deposit Designation number 201708001. Biologically pure cultures of two Klebsiella variicola variants/remodeled strains were deposited on Dec. 20, 2017 with the NCMA, located at 60 Bigelow Drive, East Boothbay, Maine 04544, USA, and assigned NCMA Patent Deposit Designation numbers 201712001 and 201712002, respectively. The applicable deposit information is found below in Table 1.

A biologically pure culture of Kosokonia sacchari was deposited on Mar. 25, 2020, with the ATCC, Manassas, VA, USA, and assigned ATTC Patent Deposit Designation number PTA-126743. This deposit was made under the provisions of the Budapest Treaty. The applicable deposit information is found below in Table 1.

A biologically pure culture of Klebsiella variicola was deposited on Mar. 25, 2020, with the ATCC, Manassas, VA, USA, and assigned ATTC Patent Deposit Designation number PTA-126741. This deposit was made under the provisions of the Budapest Treaty. The applicable deposit information is found below in Table 1.

A biologically pure culture of Klebsiella variicola was deposited on Mar. 25, 2020, with the ATCC, Manassas, VA, USA, and assigned ATTC Patent Deposit Designation number PTA-126740. This deposit was made under the provisions of the Budapest Treaty. The applicable deposit information is found below in Table 1.

A biologically pure culture of Klebsiella variicola was deposited on Apr. 2, 2020, with the ATCC, Manassas, VA, USA, and assigned ATTC Patent Deposit Designation number PTA-126749. This deposit was made under the provisions of the Budapest Treaty. The applicable deposit information is found below in Table 1.

A biologically pure culture of Herbaspirillum seropedicae was deposited on Jan. 14, 2020, with the ATCC, Manassas, VA, USA, and assigned ATTC Patent Deposit Designation number PTA-126611. This deposit was made under the provisions of the Budapest Treaty. The applicable deposit information is found below in Table 1.

TABLE 1
Microorganisms Deposited under the Budapest Treaty

| | Strain | | | |
|---|---|---|---|---|
| | Designation | | | |
| | (some strains | | | |
| | have multiple | | Accession | |
| Depository | designations) | Taxonomy | Number | Date of Deposit |
| NCMA | CI006, PBC6.1, | Kosakonia sacchari (WT) | 201701001 | Jan. 6, 2017 |
| | 6 | | | |
| NCMA | CI019, | Rahnella aquatilis (WT) | 201701003 | Jan. 6, 2017 |
| | 19 | | | |
| NCMA | CM029, 6-412 | Kosakonia sacchari | 201701002 | Jan. 6, 2017 |
| NCMA | 6-403 | Kosakonia sacchari | 201708004 | Aug. 11, 2017 |
| | CM037 | | | |
| NCMA | 6-404, | Kosakonia sacchari | 201708003 | Aug. 11, 2017 |
| | CM38, | | | |
| | PBC6.38 | | | |
| NCMA | CM094, 6-881, | Kosakonia sacchari | 201708002 | Aug. 11, 2017 |
| | PBC6.94 | | | |
| NCMA | CI137, 137, | Klebsiella variicola (WT) | 201708001 | Aug. 11, 2017 |
| | PB137 | | | |
| NCMA | 137-1034 | Klebsiella variicola | 201712001 | Dec. 20, 2017 |
| NCMA | 137-1036 | Klebsiella variicola | 201712002 | Dec. 20, 2017 |
| ATCC | 137-3890 | Klebsiella variicola | PTA-126749 | Apr. 2, 2020 |
| ATCC | 6-5687 | Kosakonia sacchari | PTA-126743 | Mar. 25, 2020 |
| ATCC | 137-3896 | Klebsiella variicola | PTA-126741 | Mar. 25, 2020 |
| ATCC | 137-2253 | Klebsiella variicola | PTA-126740 | Mar. 25, 2020 |
| ATCC | 8 | Paraburkholderia tropica | PTA-126582 | Dec. 26, 2019 |
| ATCC | 3000 | Herbaspirillum | PTA-126611 | Jan. 14, 2020 |
| | | seropedicae | | |
| DSMZ | 1666¹ | Azospirillum lipoferum | LMG13128 | |
| ATCC | 1666-7194 | Azospirillum lipoferum | PTA-127320 | Jun. 17, 2022 |
| | | PcspJ_nifA-K23E | | |
| ATCC | 1666-7481 | Azospirillum lipoferum | PTA-127323 | Jun. 17, 2022 |
| | | PcspA5_nifA- | | |
| | | K23D, M164I | | |
| ATCC | 8-5659 | Paraburkholderia tropica | PTA-127322 | Jun. 17, 2022 |
| | | PrpsL.ν3-nifA(N42D | | |
| | | D121A T166A) | | |
| ATCC | 8-5669 | Paraburkholderia tropica | PTA-127321 | Jun. 17, 2022 |
| | | PrpsL.v3-nifA(N42D | | |
| | | D121A T166A) | | |
| | | glnD_ΔUTase | | |
| ATCC | 3044 | Paraburkholderia | PTA-127324 | Jun. 17, 2022 |
| | | xenovorans WT | | |
| ATCC | 3044-6408 | Paraburkholderia | PTA-127325 | Jun. 17, 2022 |
| | | xenovorans | | |
| | | P(cspD1)-nifA_ΔGAF | | |
| ATCC | 3044-7244 | Paraburkholderia | PTA-127319 | Jun. 17, 2022 |
| | | xenovorans | | |
| | | P(cspD1)-nifA_K21E | | |

¹The corresponding wildtype strain is Azospirillum lipoferum CCUG 56042 (BacDive) (bacdive.dsmz.de/strain/137718 website).

### Isolated and Biologically Pure Microorganisms

The present disclosure, in some embodiments, provides isolated and biologically pure microorganisms that have applications, inter alia, in agriculture. The disclosed microorganisms can be utilized in their isolated and biologically pure states, as well as being formulated into compositions (see below section for exemplary composition descriptions). Furthermore, the disclosure provides microbial compositions containing at least two members of the disclosed isolated and biologically pure microorganisms, as well as methods of utilizing said microbial compositions. Furthermore, the disclosure provides for methods of modulating nitrogen fixation in plants via the utilization of the disclosed isolated and biologically pure microbes.

In some embodiments, the isolated and biologically pure microorganisms of the disclosure are those from Table 1. In some embodiments, the isolated and biologically pure microorganisms of the disclosure are derived from a microorganism of Table 1. For example, a strain, child, mutant, or derivative, of a microorganism from Table 1 are provided herein. The disclosure contemplates all possible combinations of microbes listed in Table 1, said combinations sometimes forming a microbial consortia. The microbes from Table 1, either individually or in any combination, can be combined with any plant, active molecule (synthetic, organic, etc.), adjuvant, carrier, supplement, or biological, mentioned in the disclosure.

### II. Agricultural Compositions

Compositions comprising bacteria or bacterial populations produced according to methods described herein and/or having characteristics as described herein can be in the form of a liquid, a foam, or a dry product. Compositions comprising bacteria or bacterial populations produced according to methods described herein and/or having characteristics as described herein can also be used to improve plant traits. In some examples, a composition comprising bacterial populations may be in the form of a dry powder, a slurry of powder and water, or a flowable seed treatment. The compositions comprising bacterial populations my be coated on a surface of a seed, and may be in liquid form

In some embodiments, wherein a plant, a part of the plant, or soil into which the plant is planted is contacted with more than one plurality of genetically engineered bacteria, the different pluralities of genetically engineered bacteria can be formulated separately or together. In some embodiments, wherein the different pluralities of genetically engineered bacteria are in the same composition, the composition is in the form of a liquid, a foam, or a dry product. In some embodiments, wherein the different pluralities of genetically engineered bacteria are in separate compositions (e.g., each plurality is part of a different composition), each composition is in the form of a liquid, a foam, or a dry product. In some embodiments, wherein the different pluralities of genetically engineered bacteria are in separate compositions, the first composition is in the form of a liquid and the second composition is in the form of a dry product. In some embodiments, wherein the different pluralities of genetically engineered bacteria are in separate compositions, the compositions are mixed prior to contacting a plant, a part of the plant, or soil into which the plant is planted.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Acidovorax, Agrobacterium, Bacillus, Burkholderia, Chryseobacterium, Curtobacterium, Enterobacter, Escherichia, Methylobacterium, Paenibacillus, Pantoea, Pseudomonas, Ralstonia, Saccharibacillus, Sphingomonas, Stenotrophomonas, Azospirillum, Paraburkholderia, and Herbaspirillum.

In some examples, at least two, at least three, at least four, at least five, at least six, at least seven, at least eight, at least nine, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, Pleosporaceae, Azospirillum, Paraburkholderia, and Herbaspirillum.

In some examples, at least two, at least three, at least four, at least five, at least six, at least seven, at least eight, at least night, at least ten, or more than ten bacteria and bacterial populations of an epiphytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, Pleosporaceae, Azospirillum, Paraburkholderia, and Herbaspirillum.

In some examples, at least two, at least three, at least four, at least five, at least six, at least seven, at least eight, at least night, at least ten, or more than ten bacteria and bacterial populations of a rhizospheric combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, Pleosporaceae, Azospirillum, Paraburkholderia, and Herbaspirillum.

In some embodiments, strains that can be utilized in this process of increasing colonization can include, but are not limited to, Paraburkholderia tropica, Paraburkholderia xenovorans, Azospirillum lipoferum, Rahnella aquatilis, Kosakonia sacchari, and Klebsiella variicola strains.

The compositions comprising the bacterial populations described herein may be coated onto the surface of a seed. Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10⁸ to 10¹⁰ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier. The compositions comprising the bacterial populations described herein can improve plant traits, such as promoting plant growth, maintaining high chlorophyll content in leaves, increasing fruit or seed numbers, and increasing fruit or seed unit weight.

Compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, a pesticide, including a non-naturally occurring pesticide, or a biorational or biological pesticide, an herbicide, a nematicide, an insecticide, a plant growth regulator, a fertilizer, a rodenticide, a dessicant, a bactericide, a nutrient, or any combination thereof.

In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, bacteria are mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, canola, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

In some cases, a bacterial composition can include one or more pesticides. Suitable pesticides can target economically important agronomic, forest, greenhouse, nursery ornamentals, food and fiber, public and animal health, domestic and commercial structure, household, or stored product pests. For example, the one or more pesticides can target insects, fungi, bacteria, nematodes, mites, ticks and the like. Insect pests include insects selected from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera and Coleoptera. In some cases, a biorational pesticide can be used. Such biorational pesticides include (1) biochemicals (hormones, enzymes, pheromones and natural agents, such as insect and plant growth regulators), (2) microbial (viruses, bacteria, fungi, protozoa, and nematodes), or (3) Plant-Incorporated protectants (PIPs)—primarily transgenic plants, e.g., Bt corn.

Bacteria, fungi, oomycetes, viruses and protozoa are all used for the biological control of insect pests. The most widely used microbial biopesticide is the insect pathogenic bacteria Bacillus thuringiensis (Bt), which produces a protein crystal (the Bt 8-endotoxin) during bacterial spore formation that is capable of causing lysis of gut cells when consumed by susceptible insects. Microbial Bt biopesticides consist of bacterial spores and 8-endotoxin crystals mass-produced in fermentation tanks and formulated as a sprayable product. Bt does not harm vertebrates and is safe to people, beneficial organisms and the environment. Thus, Bt sprays are a growing tactic for pest management on fruit and vegetable crops where their high level of selectivity and safety are considered desirable, and where resistance to synthetic chemical insecticides is a problem Bt sprays have also been used on commodity crops such as maize, soybean and cotton, but with the advent of genetic modification of plants, farmers are increasingly growing Bt transgenic crop varieties.

In some embodiments, fungicidal compositions may be included in the compositions set forth herein, and can be applied to a plant(s) or a part(s) thereof simultaneously or in succession, with other compounds. In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil. In some examples, a fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in a seed composition. In some cases, a fungicide can be azoxystrobin, captan, carboxin, ethaboxam, fludioxonil, mefenoxam, fludioxonil, thiabendazole, thiabendaz, ipconazole, mancozeb, cyazofamid, zoxamide, metalaxyl, PCNB, metaconazole, pyraclostrobin, Bacillus subtilis strain QST 713, sedaxane, thiamethoxam, fludioxonil, thiram, tolclofos-methyl, trifloxystrobin, Bacillus subtilis strain MBI 600, pyraclostrobin, fluoxastrobin, Bacillus pumilus strain QST 2808, chlorothalonil, copper, flutriafol, fluxapyroxad, mancozeb, gludioxonil, penthiopyrad, triazole, propiconaozole, prothioconazole, tebuconazole, fluoxastrobin, pyraclostrobin, picoxystrobin, qols, tetraconazole, trifloxystrobin, cyproconazole, flutriafol, SDHI, EBDCs, sedaxane, MAXIM QUATTRO (gludioxonil, mefenoxam, azoxystrobin, and thiabendaz), RAXIL (tebuconazole, prothioconazole, metalaxyl, and ethoxylated tallow alkyl amines), or benzovindiflupyr.

In some examples, the seed coating composition comprises a control agent which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizalfungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

Compositions comprising bacteria as described herein can include one or more herbicides. In some embodiments, herbicidal compositions are applied to the plants and/or plant parts. In some embodiments, herbicidal compositions may be included in the compositions set forth herein, and can be applied to a plant(s) or a part(s) thereof simultaneously or in succession, with other compounds. Herbicides can include 2,4-D, 2,4-DB, acetochlor, acifluorfen, alachlor, ametryn, atrazine, aminopyralid, benefin, bensulfuron, bensulide, bentazon, bicyclopyrone, bromacil, bromoxynil, butylate, carfentrazone, chlorimuron, chlorsulfuron, clethodim, clomazone, clopyralid, cloransulam, cycloate, DCPA, desmedipham, dicamba, dichlobenil, diclofop, diclosulam, diflufenzopyr, dimethenamid, diquat, diuron, DSMA, endothall, EPTC, ethalfluralin, ethofumesate, fenoxaprop, fluazifop-P, flucarbzone, flufenacet, flumetsulam, flumiclorac, flumioxazin, fluometuron, fluroxypyr, fomesafen, foramsulfuron, glufosinate, glyphosate, halosulfuron, hexazinone, imazamethabenz, imazamox, imazapic, imazaquin, imazethapyr, isoxaflutole, lactofen, linuron, MCPA, MCPB, mesotrione, metolachlor-s, metribuzin, indaziflam, metsulfuron, molinate, MSMA, napropamide, naptalam, nicosulfuron, norflurazon, oryzalin, oxadiazon, oxyfluorfen, paraquat, pelargonic acid, pendimethalin, phenmedipham, picloram, primisulfuron, prodiamine, prometryn, pronamide, propanil, prosulfuron, pyrazon, pyrithioac, quinclorac, quizalofop, rimsulfuron, S-metolachlor, sethoxydim, siduron, simazine, sulfentrazone, sulfometuron, sulfosulfuron, tebuthiuron, tembotrione, terbacil, thiazopyr, thifensulfuron, thiobencarb, topramezone, tralkoxydim, triallate, triasulfuron, tribenuron, triclopyr, trifluralin, and triflusulfuron. Herbicidal products may include CORVUS, BALANCE FLEXX, CAPRENO, DIFLEXX, LIBERTY, LAUDIS, AUTUMN SUPER, and DIFLEXX DUO.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

In some embodiments, the pesticides/microbial combinations can be applied in the form of compositions and can be applied to the crop area or plant to be treated, simultaneously or in succession, with other compounds. These compounds can be fertilizers, weed killers, cryoprotectants, surfactants, detergents, pesticidal soaps, dormant oils, polymers, and/or time release or biodegradable carrier formulations that permit long term dosing of a target area following a single application of the formulation. They can also be selective herbicides, chemical insecticides, virucides, microbicides, amoebicides, pesticides, fungicides, bacteriocides, nematicides, molluscicides or mixtures of several of these preparations, if desired, together with further agriculturally acceptable carriers, surfactants or application promoting adjuvants customarily employed in the art of formulation. Suitable carriers (i.e. agriculturally acceptable carriers) and adjuvants can be solid or liquid and correspond to the substances ordinarily employed in formulation technology, e.g. natural or regenerated mineral substances, solvents, dispersants, wetting agents, sticking agents, tackifiers, binders or fertilizers. Likewise, the formulations may be prepared into edible baits or fashioned into pest traps to permit feeding or ingestion by a target pest of the pesticidal formulation.

In some cases, a composition provided here can include a microbial insecticide based on entomopathogenic baculoviruses. Baculoviruses that are pathogenic to arthropods belong to the virus family and possess large circular, covalently closed, and double-stranded DNA genomes that are packaged into nucleocapsids. More than 700 baculoviruses have been identified from insects of the orders Lepidoptera, Hymenoptera, and Diptera. Baculoviruses are usually highly specific to their host insects and thus, are safe to the environment, humans, other plants, and beneficial organisms. Over 50 baculovirus products have been used to control different insect pests worldwide. In the US and Europe, the Cydia pomonella granulovirus (CpGV) is used as an inundative biopesticide against codlingmoth on apples. Washington State, as the biggest apple producer in the US, uses CpGV on 13% of the apple crop. In Brazil, the nucleopolyhedrovirus of the soybean caterpillar Anticarsia gemmatalis was used on up to 4 million ha (approximately 35%) of the soybean crop in the mid-1990s. Viruses such as Gemstar® (Certis USA) are available to control larvae of Heliothis and Helicoverpa species.

At least 170 different biopesticide products based on entomopathogenic fungi have been developed for use against at least five insect and acarine orders in glasshouse crops, fruit and field vegetables as well as commodity crops. The majority of products are based on the ascomycetes Beauveria bassiana or Metarhizium anisopliae. M anisopliae has also been developed for the control of locust and grasshopper pests in Africa and Australia and is recommended by the Food and Agriculture Organization of the United Nations (FAO) for locust management.

A number of microbial pesticides registered in the United States are listed in Table 16 of Kabaluk et al. 2010 (Kabaluk, J. T. et al. (ed.). 2010. The Use and Regulation of Microbial Pesticides in Representative Jurisdictions Worldwide. IOBC Global. 99pp.) and microbial pesticides registered in selected countries are listed in Annex 4 of Hoeschle-Zeledon et al. 2013 (Hoeschle-Zeledon, I., P. Neuenschwander and L. Kumar. (2013). Regulatory Challenges for biological control. SP-IPM Secretariat, International Institute of Tropical Agriculture (IITA), Ibadan, Nigeria. 43 pp.), each of which is incorporated herein in its entirety.

Plants produce a wide variety of secondary metabolites that deter herbivores from feeding on them. Some of these can be used as biopesticides. They include, for example, pyrethrins, which are fast-acting insecticidal compounds produced by Chrysanthemum cinerariaefolium. They have low mammalian toxicity but degrade rapidly after application. This short persistence prompted the development of synthetic pyrethrins (pyrethroids). The most widely used botanical compound is neem oil, an insecticidal chemical extracted from seeds of Azadirachta indica. Two highly active pesticides are available based on secondary metabolites synthesized by soil actinomycetes, but they have been evaluated by regulatory authorities as ifthey were synthetic chemical pesticides. Spinosad is a mixture oftwo macrolide compounds from Saccharopolyspora spinosa. It has a very low mammalian toxicity and residues degrade rapidly in the field. Farmers and growers used it widely following its introduction in 1997 but resistance has already developed in some important pests such as western flower thrips. Abamectin is a macrocyclic lactone compound produced by Streptomyces avermitilis. It is active against a range of pest species but resistance has developed to it also, for example, in tetranychid mites.

Peptides and proteins from a number of organisms have been found to possess pesticidal properties. Perhaps most prominent are peptides from spider venom (King, G. F. and Hardy, M. C. (2013) Spider-venom peptides: structure, pharmacology, and potential for control of insect pests. Annu. Rev. Entomol. 58: 475-496). A unique arrangement of disulfide bonds in spider venom peptides render them extremely resistant to proteases. As a result, these peptides are highly stable in the insect gut and hemolymph and many of them are orally active. The peptides target a wide range of receptors and ion channels in the insect nervous system Other examples of insecticidal peptides include: sea anemone venom that act on voltage-gated Na+ channels (Bosmans, F. and Tytgat, J. (2007) Sea anemone venom as a source of insecticidal peptides acting on voltage-gated Na+ channels. Toxicon. 49(4): 550-560); the PAlb (Pea Albumin 1, subunit b) peptide from Legume seeds with lethal activity on several insect pests, such as mosquitoes, some aphids and cereal weevils (Eyraud, V. et al. (2013) Expression and Biological Activity of the Cystine Knot Bioinsecticide PAlb (Pea Albumin 1 Subunit b). PLoS ONE 8(12): e81619); and an internal 10 kDa peptide generated by enzymatic hydrolysis of Canavalia ensiformis Gack bean) urease within susceptible insects (Martinelli, A. H. S., et al. (2014) Structure-function studies on jaburetox, a recombinant insecticidal peptide derived from jack bean (Canavalia ensiformis) urease. Biochimica et Biophysica Acta 1840: 935-944). Examples of commercially available peptide insecticides include Spear™-T for the treatment of thrips in vegetables and ornamentals in greenhouses, Spear™-P to control the Colorado Potato Beetle, and Spear™-C to protect crops from lepidopteran pests (Vestaron Corporation, Kalamazoo, MI). A novel insecticidal protein from Bacillus bombysepticus, called parasporal crystal toxin (PC), shows oral pathogenic activity and lethality towards silkworms and Cry1Ac-resistant Helicoverpa armigera strains (Lin, P. et al. (2015) PC, a novel oral insecticidal toxin from Bacillus bombysepticus involved in host lethality via APN and BtR-175. Sci. Rep. 5: 11101).

A semiochemical is a chemical signal produced by one organism that causes a behavioral change in an individual of the same or a different species. The most widely used semiochemicals for crop protection are insect sex pheromones, some of which can now be synthesized and are used for monitoring or pest control by mass trapping, lure-and-kill systems and mating disruption. Worldwide, mating disruption is used on over 660,000 ha and has been particularly useful in orchard crops.

As used herein, “transgenic insecticidal trait” refers to a trait exhibited by a plant that has been genetically engineered to express a nucleic acid or polypeptide that is detrimental to one or more pests. In one embodiment, the plants of the present disclosure are resistant to attach and/or infestation from any one or more of the pests of the present disclosure. In one embodiment, the trait comprises the expression of vegetative insecticidal proteins (VIPs) from Bacillus thuringiensis, lectins and proteinase inhibitors from plants, terpenoids, cholesterol oxidases from Streptomyces spp., insect chitinases and fungal chitinolytic enzymes, bacterial insecticidal proteins and early recognition resistance genes. In another embodiment, the trait comprises the expression of a Bacillus thuringiensis protein that is toxic to a pest. In one embodiment, the Bt protein is a Cry protein (crystal protein). Bt crops include Bt corn, Bt cotton and Bt soy. Bt toxins can be from the Cry family (see, for example, Crickmore et al., 1998, Microbiol. Mol. Biol. Rev. 62: 807-812), which are particularly effective against Lepidoptera, Coleoptera andDiptera.

Bt Cry and Cyt toxins belong to a class of bacterial toxins known as pore-forming toxins (PFT) that are secreted as water-soluble proteins undergoing conformational changes in order to insert into, or to translocate across, cell membranes of their host. There are two main groups of PFT: (i) the a-helical toxins, in which a-helix regions form the trans membrane pore, and (ii) the P-barrel toxins, that insert into the membrane by forming a P-barrel composed of Psheet hairpins from each monomer. See, Parker MW, Feil SC, “Pore-forming protein toxins: from structure to function,” Prog. Biophys. Mol. Biol. 2005 May; 88(1):91-142.

The first class of PFT includes toxins such as the colicins, exotoxin A, diphtheria toxin and also the Cry three-domain toxins. On the other hand, aerolysin, a-hemolysin, anthrax protective antigen, cholesterol-dependent toxins as the perfringolysin O and the Cyt toxins belong to the P-barrel toxins. Id. In general, PFT producing-bacteria secrete their toxins and these toxins interact with specific receptors located on the host cell surface. In most cases, PFT are activated by host proteases after receptor binding inducing the formation of an oligomeric structure that is insertion competent. Finally, membrane insertion is triggered, in most cases, by a decrease in pH that induces a molten globule state of the protein. Id.

The development oftransgenic crops that produce Bt Cry proteins has allowed the substitution of chemical insecticides by environmentally friendly alternatives. In transgenic plants the Cry toxin is produced continuously, protecting the toxin from degradation and making it reachable to chewing and boring insects. Cry protein production in plants has been improved by engineering cry genes with a plant biased codon usage, by removal of putative splicing signal sequences and deletion of the carboxy-terminal region of the protoxin. See, Schuler T H, et al., “Insect-resistant transgenic plants,” Trends Biotechnol. 1998; 16:168-175. The use of insect resistant crops has diminished considerably the use of chemical pesticides in areas where these transgenic crops are planted. See, Qaim M, Zilberman D, “Yield effects of genetically modified crops in developing countries,” Science. 2003 Feb. 7; 299(5608):900-2.

Known Cry proteins include: 8-endotoxins including but not limited to: the Cry1, Cry2, Cry3, Cry4, Cry5, Cry6, Cry7, Cry8, Cry9, Cry10, Cry1l, Cry12, Cry13, Cry14, Cry15, Cry16, Cry1 7, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51, Cry52, Cry 53, Cry 54, Cry55, Cry56, Cry57, Cry58, Cry59. Cry60, Cry61, Cry62, Cry63, Cry64, Cry65, Cry66, Cry67, Cry68, Cry69, Cry70 and Cry71 classes of 8-endotoxin genes and the B. thuringiensis cytolytic cyt1 and cyt2 genes.

Examples of 8-endotoxins also include but are not limited to Cry1A proteins of U.S. Pat. Nos. 5,880,275, 7,858,849 8,530,411, 8,575,433, and 8,686,233; a DIG-3 or DIG-11 toxin (N-terminal deletion of a-helix 1 and/or a-helix 2 variants of cry proteins such as Cry1A, Cry3 A) of U.S. Pat. Nos. 8,304,604, 8,304,605 and 8,476,226; Cry1B of U.S. patent application Ser. No. 10/525,318; Cry1C of U.S. Pat. No. 6,033,874; Cry1F of U.S. Pat. Nos. 5,188,960 and 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including butnot limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E and Cry9F families. Other Cry proteins are well known to one skilled in the art. See, N. Crickmore, et al., “Revision of the Nomenclature for the Bacillus thuringiensis Pesticidal Crystal Proteins,” Microbiology and Molecular Biology Reviews,” (1998) Vol 62: 807-813; see also, N. Crickmore, et al., “Bacillus thuringiensis toxin nomenclature” (2016), at www.btnomenclature.info/.

The use of Cry proteins as transgenic plant traits is well known to one skilled in the art and Cry-transgenic plants including but not limited to plants expressing Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval. See, Sanahuja et al., “Bacillus thuringiensis: a century of research, development and commercial applications,” (2011) Plant Biotech Journal, April 9(3):283-300 and the CERA (2010) GM Crop Database Center for Environmental Risk Assessment (CERA), ILS Research Foundation, Washington D.C. at cera gmc.org/index.php?action=gm_crop_database, which can be accessed on the world-wide web using the “www” prefix). More than one pesticidal proteins well known to one skilled in the art can also be expressed in plants such as Vip3Ab & Cry1Fa (US2012/0317682); Cry1BE & Cry1F (US2012/0311746); Cry1CA & Cry1AB (US2012/0311745); Cry1F & CryCa (US2012/0317681); Cry1DA& Cry1BE (US2012/0331590); Cry1DA & Cry1Fa (US2012/0331589); Cry1AB & Cry1BE (US2012/0324606); Cry1Fa & Cry2Aa and Cry11 & Cry1E (US2012/0324605); Cry34Ab/35Ab and Cry6Aa (US20130167269); Cry34Ab/VCry35Ab & Cry3Aa (US20130167268); Cry1Ab & Cry1F (US20140182018); and Cry3A and Cry1Ab or Vip3Aa (US20130116170). Pesticidal proteins also include insecticidal lipases including lipid acyl hydrolases of U.S. Pat. No. 7,491,869, and cholesterol oxidases such asfromStreptomyces (Purcell et al. (1993) Biochem Biophys Res Commun 15:1406-1413).

Pesticidal proteins also include VIP (vegetative insecticidal proteins) toxins. Entomopathogenic bacteria produce insecticidal proteins that accumulate in inclusion bodies or parasporal crystals (such as the aforementioned Cry and Cyt proteins), as well as insecticidal proteins that are secreted into the culture medium Among the latter are the Vip proteins, which are divided into four families according to their amino acid identity. The Vip1 and Vip2 proteins act as binary toxins and are toxic to some members of the Coleoptera and Hemiptera. The Vip1 component is thought to bind to receptors in the membrane of the insect midgut, and the Vip2 component enters the cell, where it displays its ADP-ribosyltransferase activity against actin, preventing microfilament formation. Vip3 has no sequence similarity to Vip1 or Vip2 and is toxic to a wide variety of members of the Lepidoptera. Its mode of action has been shown to resemble that of the Cry proteins in terms of proteolytic activation, binding to the midgut epithelial membrane, and pore formation, although Vip3A proteins do not share binding sites with Cry proteins. The latter property makes them good candidates to be combined with Cry proteins in transgenic plants (Bacillus thuringiensis-treated crops [Bt crops]) to prevent or delay insect resistance and to broaden the insecticidal spectrum. There are commercially grown varieties of Bt cotton and Bt maize that express the Vip3Aa protein in combination with Cry proteins. For the most recently reported Vip4 family, no target insects have been found yet. See, Chakroun et al., “Bacterial Vegetative Insecticidal Proteins (Vip) from Entomopathogenic Bacteria,” Microbiol Mol Biol Rev. 2016 Mar. 2; 80(2):329-50. VIPs can be found in U.S. Pat. Nos. 5,877,012, 6,107,279 6,137,033, 7,244,820, 7,615,686, and 8,237,020 and the like. Other VIP proteins are well known to one skilled m the art (see, lifesci.sussex.ac.uk/home/Neil_Crickmore/Bt/vip.html, which can be accessed on the world wide web using the “www” prefix).

Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins

(“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1 Wi. Examples of Class C proteins are TccC, XptC1Xb and XptB1 Wi. Pesticidal proteins also include spider, snake and scorpion venom proteins. Examples of spider venom peptides include, but are not limited to lycotoxin-1 peptides and mutants thereof (U.S. Pat. No. 8,334,366).

Transgenic plants have also been engineered to express dsRNA directed against insect genes (Baum, J. A. et al. (2007) Control of coleopteran insect pests through RNA interference. Nature Biotechnology 25: 1322-1326; Mao, Y. B. et al. (2007) Silencing a cotton bollworm P450 monooxygenase gene by plant-mediated RNAi impairs larval tolerance of gossypol. Nature Biotechnology 25: 1307-1313). RNA interference can be triggered in the pest by feeding of the pest on the transgenic plant. Pest feeding thus causes injury or death to the pest.

In some embodiments, any one or more of the pesticides set forth herein may be utilized with any one or more of the microbes of the disclosure and can be applied to plants or parts thereof, including seeds.

### Application of Bacterial Populations on Crops

The composition of the bacteria or bacterial population described herein can be applied in furrow, in talc, or as seed treatment. The composition can be applied to a seed package in bulk, mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, sorghum, potato, rice, vegetables, cereals, pseudocereals, and oilseeds. Examples of cereals may include barley, fonio, oats, palmer's grass, rye, pearl millet, sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition may improve plant traits, such as promoting plant growth, maintaining high chlorophyll content in leaves, increasing fruit or seed numbers, and increasing fruit or seed unit weight. Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation.

In some cases, plants are inoculated with bacteria or bacterial populations that are isolated from the same species of plant as the plant element of the inoculated plant. For example, an bacterium or bacterial population that is normally found in one variety of Zea mays (corn) is associated with a plant element of a plant of another variety of Zea mays that in its natural state lacks said bacteria and bacterial populations. In one embodiment, the bacteria and bacterial populations is derived from a plant of a related species of plant as the plant element of the inoculated plant. For example, an bacteria and bacterial populations that is normally found in Zea diploperennis Iltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, an bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present invention's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal 5 root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In certain embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The CHU can also be used to predict the maturation of the corn crop. The CHU determines the amount of heat accumulation by measuring the daily maximum temperatures on crop growth.

In examples, bacterial may localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, and asparagus. In some embodiments, the methods and bacteria described herein are suitable for any of a variety of transgenic plants, non-transgenic plants, and hybrid plants thereof.

In some examples, plants that may be obtained or improved using the methods and composition disclosed herein may include plants that are important or interesting for agriculture, horticulture, biomass for the production of biofuel molecules and other chemicals, and/or forestry. Some examples of these plants may include pineapple, banana, coconut, lily, grasspeas, and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum spp., Sorghum bicolor (sorghum, sudan), Miscanthus spp., Miscanthus giganteus (miscanthus), Saccharum spp. (energycane), Populus balsamifera (poplar), Zea mays (corn), Glycine max (soybean), Brassica napus (canola), Brassica juncea, Brassica oleracea (broccoli, cauliflower, brussel sprouts), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Manihot esculenta (cassaya), Lycopersicon esculentum (tomato), Lactuca saliva (lettuce), Musa paradisiaca (banana), Solanum tuberosum (potato), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to a plant of another type (e.g. a crop plant) to confirm conferral of the improved trait.

Traits that may be improved by the methods disclosed herein include any observable characteristic of the plant, including, for example, growth rate, height, weight, color, taste, smell, changes in the production of one or more compounds by the plant (including for example, metabolites, proteins, drugs, carbohydrates, oils, and any other compounds). Selecting plants based on genotypic information is also envisaged (for example, including the pattern of plant gene expression in response to the bacteria, or identifying the presence of genetic markers, such as those associated with increased nitrogen fixation). Plants may also be selected based on the absence, suppression or inhibition of a certain feature or trait (such as an undesirable feature or trait) as opposed to the presence of a certain feature or trait (such as a desirable feature or trait).

The invention will be further described in the following examples, which do not limit the scope of the invention described in the claims.

### EXAMPLES

### Example 1—Creating a nifA Mutant Library and Development of a Screening Method to Identify New nifA Variants

New variants of NifA that lack self-inhibition in nitrogen-rich conditions as well as oxygen-resistant versions of NifA were developed in Paraburkholderia and Azospirillum. Self-inhibition refers to the N-terminal GAF domain folding back onto the central AAA domain and inhibiting its own activity to induce transcription of downstream genes, under nitrogen-rich conditions. Under nitrogen-limited conditions, wild-type nifA does not inhibit itself. Mutations described in these examples can prevent the self-inhibition.

In the diazotrophs where an anti-activator NifL is lacking, the expression of nif genes is repressed by ammonium via NifA. The NifA protein is composed of an N-terminal GAF domain, an AAA⁺ ATPase domain, and a C-terminal DNA binding domain.

A platform to quantify expression of the nif genes and screen a large mutant library for constitutive nitrogenase expression in two genera (Paraburkholderia and Azospirillum) was developed. nifH expression was visualized by fluorescence output. In order to assess nitrogenase expression, the nifH promoter was placed upstream of the fluorescence reporter gene GFP. nifH promoter activation was tested in the presence of ammonium and oxygen. Directed evolution of the regions sensing nitrogen status in NifA was used to generate mutants that were resistant to ammonium and oxygen. Selected NifA variants were introduced into the genome of Paraburkholderia and Azospirillum. Ammonium sensitivity of nitrogenase was assessed by an in vitro nitrogenase assay. Selected mutants showed nitrogenase activity regardless of ammonium availability.

### Bacterial Strains and Growth Media

E. coli DH10-beta (New England Biolabs) was used for cloning. For rich media, LB medium were used for E. coli and SOB medium was used for Paraburkholderia and Azospirillum. For minimal media, Paraburkholderia minimal medium (0.6 g/L K₂HPO₄, 1.8 g/L KH₂PO₄, 5 g/L mannitol, 0.1 g/L CaCl₂)×2H₂O, 0.1 g/L NaCl, 0.2 g/L MgSO₄×7H₂O, 66 mg/L Fe-EDTA, 0.04 mg/L CuSO₄×5H₂O, 0.12 mg ZnSO₄×7H₂O, 1.4 mg/L H₃BO₄, 1.175 mg/L MnSO₄, 1 mg/L Na₂MoO₄×2H₂O, pH 7.0) was used for Paraburkholderia. Azospirillum minimal medium (0.9 g/L K₂HPO₄, 0.6 g/L KH₂PO₄, 0.4 g/L MgSO₄×7H₂O, 0.05 g/L CaCl₂)×2H₂O, 0.2 g/L NaCl, 4 mg/L Na₂MoO₄×2H₂O, 0.015 g/L MnSO₄, 1.2636 g/L FeSO₄×7H₂O, 1.184 g/L EDTA, pH 7.0) was used for Azospirillum. Antibiotics were used at the following concentrations: E. coli, Paraburkholderia and Azospirillum (gentamicin, 15 μg/mL).

### Development of a High-Throughput Screening System for Screening Ammonium Tolerant NifA in Paraburkholderia

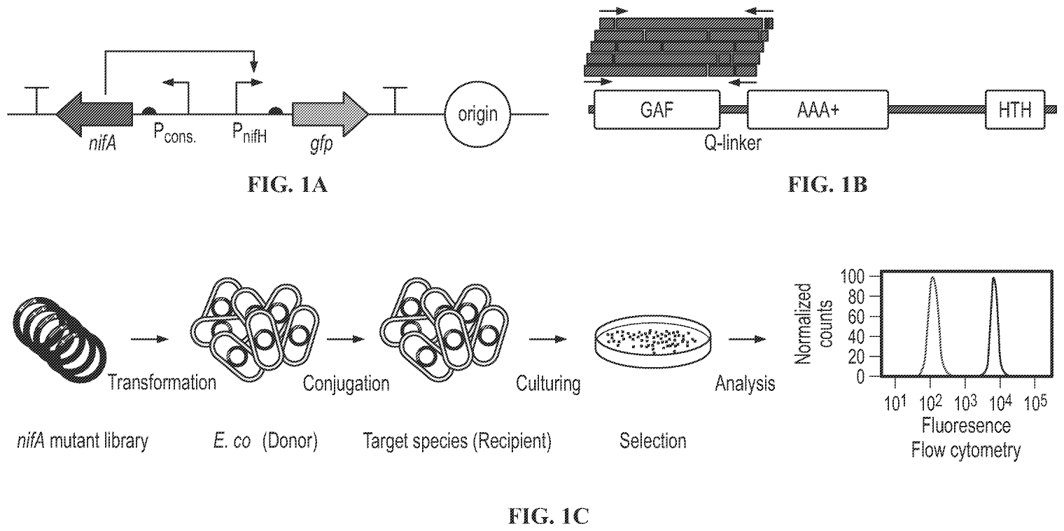

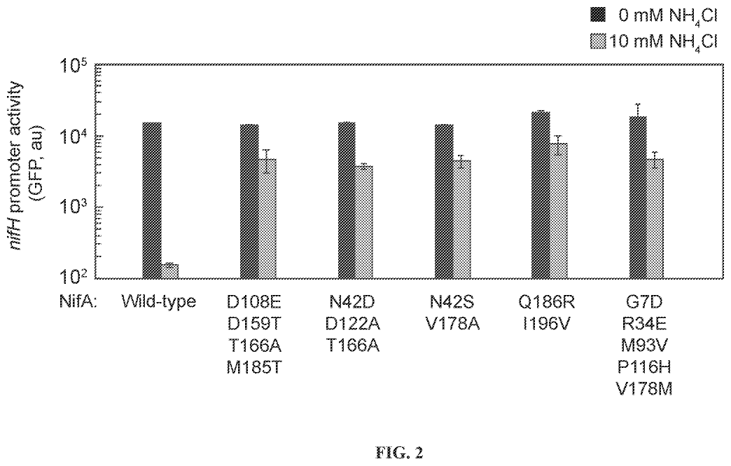

To identify NifA variants that remove ammonium repression, the genomic copy of nifA from Paraburkholderia strain CI8 was deleted and a reporter plasmid with a nifA gene was inserted into the NifA deficient CI8 strain. The reporter plasmid was constructed by amplifying the nifH promoter and nifA gene from genomic DNA of CI8 and inserting the fragment upstream of the gene sequence encoding a green fluorescent protein (GFP) and downstream of the constitutive T7 wild-type promoter, respectively, in a plasmid based on dual origins (p15a for E. coli and pRO1600 for Paraburkholderia). The gene encoding GFP is operably linked to the nifH promoter (FIG. 1a). The reporter plasmid also contained the RK2 origin of transfer (oriT) in order to enable conjugative transfer from E. coli to Paraburkholderia. Triparental mating was then used to transfer DNA from E. coli to the Paraburkholderia strain lacking nufA. An aliquot of 60 μl of late-log phase donor cells and 60 μl of late-log phase helper cells containing a helper plasmid that allowed conjugative delivery of the reporter plasmid in donor cells were mixed with 100 μl of late-log phase recipient Paraburkholderia cells lacking nifA and washed with 200 μl of SOB medium. Mating was initiated by spotting 20 μl of the mixed cells on SOB agar plates and incubated at 30° C. for 3 hr. The mating mixtures were plated on SOB agar plates supplemented with 10 μg/ml nitrofurantoin to kill E. coli and 7.5 μg/ml gentamicin to select plasmid transfer.

Subsequently, the ability of the system to recapitulate the native regulation of the nifH promoter with NifA complementation was assessed by expressing wild-type nifA on the same reporter plasmid (FIG. 1a). To test control of the expression of the nif cluster in response to ammonium, the nifH promoter activity was analyzed using flow cytometry. Single colonies were inoculated into 0.5 ml SOB medium supplemented with 7.5 μg/ml gentamicin in 96-deep-well plates and incubated overnight at 30° C. and 900 r.p.m. Aliquots (2 μl) of the overnight cultures were diluted in 200 μl Paraburkholderia minimal medium containing 10 mM ammonium chloride and 7.5 μg/ml gentamicin in 96-well plates, and incubated for 24 hr at 30° C. and 900 r.p.m. Aliquots (1.5 μl) of the cultures were diluted in 100 μl Paraburkholderia minimal medium containing 7.5 μg/ml gentamicin with or without the addition of 10 mM ammonium chloride in 96-well plates and incubated for 15 hr at 30° C. and 800 r.p.m at 1% oxygen. Aliquots (4 μl) of these cultures were then diluted in 150 μl PBS with 2 mg/ml kanamycin for flow cytometry analysis. Cultures with fluorescence proteins were analyzed by flow cytometry using an Attune Nx/T Flow Cytometer with a 488 nm laser and 510/20-nm band-pass filter for GFP. The cells were collected over 10,000 events, which were gated using forward and side scatter to remove background events using FlowJo (TreeStar Inc.). The median fluorescence from the cytometry histograms was calculated for all samples. The median autofluorescence was subtracted from the median fluorescence and reported as the fluorescence value in arbitrary units. The nifH expression was decreased by two orders of magnitude with the addition of 10 mM ammonium, indicating that the system can be adapted to select NifA mutants that do not repress the nufgene expression in the presence of ammonium while maintaining their activity regardless of ammonium availability (FIG. 2).

Identification of Ammonium Tolerant NifA in Paraburkholderia from Directed Evolution

In order to generate potentially ammonium resistant nifA mutants, the GAF domain and Q-linker of nifA was amplified from the genomic DNA of CI8 by error-prone PCR and inserted into another plasmid with a pRO1600 origin (FIG. 1b). The error-prone PCR utilized PCR reactions with 1×PCR buffer supplemented with 7 mM MgSO₄, 0.4 mM MnSO₄, 1 mM dNTP and 0.05 U GoTaq® DNA polymerase (Promega). Furthermore the nifA mutants generated from the error-prone PCR were cloned into the plasmids that also contained the RK2 origin of transfer (oriT) to enable the conjugative transfer from E. coli to Paraburkholderia. nufA variants were collected to a library size of 10⁶ recombinants in E. coli. The pooled nifA libraries were introduced into CI8 in a manner similar to that described for the reporter plasmid (FIG. 1c). Triparental mating was used to transfer DNA from E. coli to Paraburkholderia. An aliquot of 60 μl of late-log phase donor cells and 60 μl of late-log phase helper cells containing a helper plasmid that allowed conjugative delivery of a nifA library in donor cells were mixed with 100 μl of late-log phase recipient Paraburkholderia cells and washed with 200 μl of SOB medium. Mating was initiated by spotting 20 μl of the mixed cells on SOB agar plates and incubated at 30° C. for 3 hr. The mating mixtures were plated on the minimal agar plates supplemented with 10 μg/ml nitrofurantoin, 7.5 μg/ml gentamicin and 10 mM ammonium chloride and incubated at 30° C. for 5 days under hypoxic conditions (1% oxygen). Derepression of the nif cluster was visualized by GFP expression and colonies showing induction of the nifH promoter arose at a frequency of ˜10⁻⁴.

After isolation of the GFP expressing colonies from the agar plates, the nifH promoter induction with nufA variants was compared to the induction of the wild-type nifA and substituted residues were identified by sequencing (FIG. 1c). A total of 26 nifA variants that recover at least 10% of nifH promoter activity in the presence of ammonium was identified by flow cytometry. The mutant residues identified in more than one variant are listed in Table 5 with their occurrence number from the active NifA mutants. Five NifA variants, including [D108E, D159T, T166A, and M185T], [N42D, D122A, and T166A], [N42S and V178A], [Q186R and I196V], and [G7D, R34E, M193V, P166H, and V178M], were selected that recovered more than 25% of nifH promoter activity in the presence of ammonium for further analysis (FIG. 2).

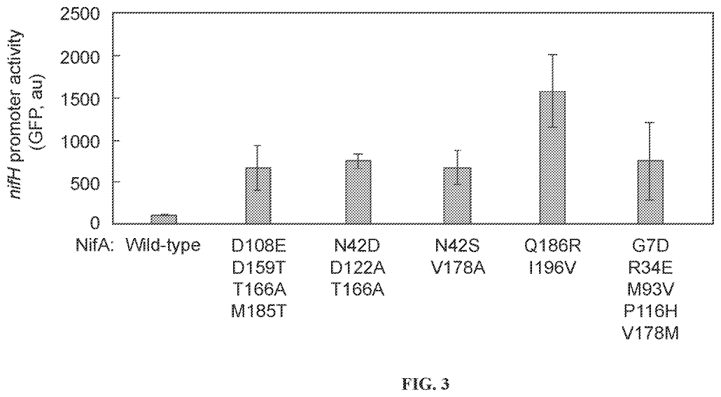

To compare oxygen tolerance of these nifA variants, the nifH promoter activity was analyzed in the presence of atmospheric oxygen (20% oxygen) and ammonium (FIG. 3). Single colonies were inoculated into 0.5 ml SOB medium supplemented with 7.5 μg/ml gentamicin in 96-deep-well plates and incubated overnight at 30° C. and 900 r.p.m. Aliquots (2 μl) of the overnight cultures were diluted in 200 μl Paraburkholderia minimal medium containing 10 mM ammonium chloride and 7.5 μg/ml gentamicin in 96-well plates, and incubated for 24 hr at 30° C. and 900 r.p.m. Aliquots (4 μl) of these cultures were then diluted in 150 e PBS with 2 mg/mi kanamycin for flow cytometry analysis. Cultures with fluorescence proteins were analyzed by flow cytometry as described above. The nifH expression increased by 15-fold in the NifA variant comprising Q186R and I196V substitutions compared to the wild-type NifA. Engineered P. tropica strains are described in Table 2.

TABLE 2
Paraburkholderia tropica (CI8) strains engineered for derepression of nifA.

| | | ARA | ARA | |
|---|---|---|---|---|
| | | Ethylene | Ethylene | |
| | | in 0 mM | in 10 mM | |
| | | NH4Cl | NH4Cl | [NH4+] |
| Strain ID | Genotype | (mm/OD) | (mm/OD) | (mM) |
| 8 | Wild-type | 0.037E−13 | 0 | 0-0.046 |
| 8-3916 | ΔnifH | 0 | 0 | 0-0.01 |
| 8-4530 | ΔP(nifA)_ν2::P(acnB)-nifA_ΔGAF | 0 | 0 | N/A |
| 8-4536 | ΔP(nifA)_ν2::P(ppsA)-nifA_ΔGAF | 0 | 0 | N/A |
| 8-4542 | ΔP(nifA)_ν2::P(rpoBC)-nifA_ΔGAF | 0 | 0 | N/A |
| 8-4546 | ΔP(nifA)_ν2::P(infC) | 0.008E−13 | 0 | N/A |
| 8-4548 | ΔP(nifA)_ν1::P(rpsL) | 0.022E−13- | 0 | 0 |
| | | 0.234E−13 | | |
| 8-4550 | ΔP(nifA)_ν2::P(rpsL) | 0.027E−13- | 0 | 0 |
| | | 0.450E−13 | | |
| 8-4554 | ΔP(nifA)_ν2::P(tufA2) | 0.015E−13 | 0 | N/A |
| 8-4558 | ΔP(nifA)_ν2::P(acnB) | 0 | 0 | N/A |
| 8-4560 | ΔP(nifA)_ν2::P(rpoBC) | 0.002E−13 | 0 | N/A |
| 8-4562 | glnE_ΔAR | 0.006E−13- | 0-0.001E−13 | 0-0.007 |
| | | 0.029E−13 | | |
| 8-4564 | ΔglnK | 0.009E−13- | 0 | 0-0.048 |
| | | 0.029E−13 | | |
| 8-4600 | ΔP(nifA)_ν2::P(rpsL)-nifA_ΔGAF | 0.0205E−13- | 0.729E−13- | 0.762-1.223 |
| | | 0.335E−13 | 0.799E−13 | |
| 8-4740 | glnD_ΔUtase | 0.003E−13- | 0-0.695E−13 | 0.012-0.710 |
| | | 1.814E−13 | | |
| 8-4784 | ΔP(nifA)_ν2::P(rpsL)-nifA_ΔGAF, | 0-0.437E−13 | 0-0.253E−13 | 0.617-2.212 |
| | glnE ΔAR | | | |
| 8-4790 | ΔP(nifA)_ν2::P(rpsL)-nifA_ΔGAF, | 0-0.728E−13 | 0-0.402E−13 | 2.529-3.067 |
| | glnD ΔUTase | | | |
| 8-5055 | P(rpsL)-nifA_ΔGAF | 0.726E−13- | 0.630E−13- | 0.165-4.395 |
| | | 2.087E−13 | 1.078E−13 | |
| 8-5364 | ΔP(nifA)_ν2::P(rpsL)-nifA_Δ(Q2-V24) | 0-0.773E−13 | 0-0.094E−13 | 0 0.031 |
| 8-5366 | ΔP(nifA)_ν2::P(rpsL)-nifA_Δ(Q2-176) | 0-1.780E−13 | 0-0.406E−13 | 0-0.034 |
| 8-5368 | ΔP(nifA)_ν2::P(rpsL)-nifA_Δ(Q2-G139) | 0-0.833E−13 | 0-0.079E−13 | 0-0.024 |
| 8-4598 | ΔP(nifA)_ν1::P(rpsL)-nifA_ΔGAF | 0-0.001E−13 | 0-0.187E−13 | 0 |
| 8-5063 | P(rpsL)-nifA_ΔGAF, glnD_ΔUTase | 1.275E−13- | 1.351E−13- | 9.745-13.307 |
| | | 3.455E−13 | 3.926E−13 | |
| 8-5657 | P(rpsL)-nifA_1B2 | 2.454E−13- | 0.691E−13- | 0-0.016 |
| | | 12.186E−13 | 3.771E−13 | |
| 8-5659 | P(rpsL)-nifA_2B2 | 1.646E−13- | 1.377E−13- | 4.452-5.530 |
| | | 7.025E−13 | 7.025E−13 | |
| 8-5661 | P(rpsL)-nifA_2D11 | 1.847E−13- | 2.626E−13- | 0.878-4.856 |
| | | 13.121E−13 | 12.590E−13 | |
| 8-5663 | P(rpsL)-nifA_3D3 | 1.413E−13- | 1.098E−13- | 2.567-4.345 |
| | | 8.224E−13 | 6.658E−13 | |
| 8-5665 | P(rpsL)-nifA_3F8r | 2.650E−13- | 1.098E−13- | 2.158-3.665 |
| | | 13.034E−13 | 13.375E−13 | |
| 8-5667 | P(rpsL)-nifA_1B2, glnD_ΔUTase | 0.798E−13 | 0.278E−13 | 0 |
| 8-5669 | P(rpsL)-nifA_2B2, glnD_ΔUTase | 2.256E−13- | 2.368E−13- | 9.831-9.959 |
| | | 4.912E−13 | 3.716E−13 | |
| 8-5671 | P(rpsL)-nifA_2D11, glnD_ΔUTase | 1.882E−13 | 2.154E−13 | 0.183-8.706 |
| 8-5673 | P(rpsL)-nifA_3D3, glnD_ΔUTase | 2.306E−13- | 2.036E−13- | 2.328-9.946 |
| | | 5.358E−13 | 4.981E−13 | |
| 8-5675 | P(rpsL)-nifA_3F8r, glnD_ΔUTase | 0.652E−13 | 1.358E−13 | 7.052-9.895 |

### Example 2—Combining nifA Domain Deletions and Point Mutations to Effect NifA Activity

In this example, new domain deletions that derepress NifA activity were identified and combined with other mutations for additive enhancement of NifA activity.

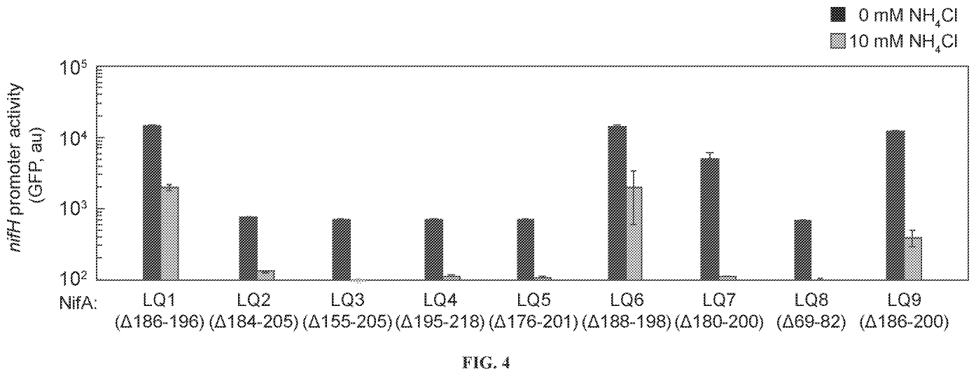

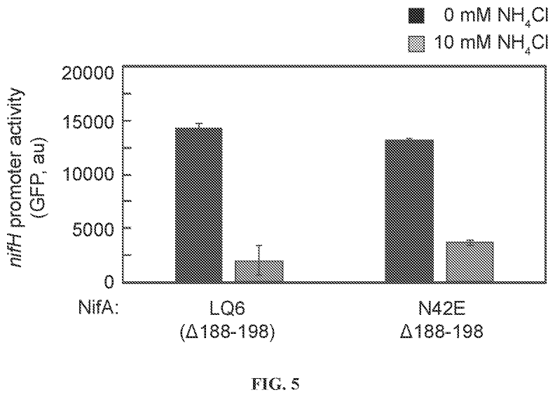

Q-linker deletions and isolated SNPs in the Q-linker region in Rhodopseudomonas palustric enabled nitrogenase expression in ammonium-grown cells (see, for example, McKinlay, J. B. and Harwood, C. S., 2010. P.N.A.S, 107(26), pp. 11669-11675.). A range of deletions in the Q-linker regions, including a deletion of residues 186-196 (mutant LQ1), 188-198 (mutant LQ6), and 186-200 (mutant LQ9), were genetically engineered into the reporter plasmid reporter based on a pRO1600 origin in which a fluorescence reporter GFP is operably linked to the nifH promoter. The reporter plasmid was transferred into a Paraburkholderia strain lacking nifA by conjugation and the nifH promoter activity was analyzed under hypoxic conditions (100 oxygen). Among a series of NifA variants containing deletions in the Q-linker region, the mutants LQ1, LQ6, and LQ9 yielded 13%, 13% and 3% of the nifH promoter activity in the presence of ammonium, respectively, compared to the full activity from the wild-type NifA in the absence of ammonium (FIG. 4).

To understand whether SNPs in the N-terminal GAF domain of NifA can contribute additively to ammonium derepression when introduced in the variants with a Q-linker deletion, the SNPs in GAF domain and the Q-linker deletions were combined. The LQ6 deletion, which recovers the highest activity in the presence of ammonium, was used as the Q-linker deletion. The substitution in the conserved residue (G25) relieved ammonium repression in Herbaspirillum (Aquino et al., 2015. Brazilian Journal of Medical and Biological Research, 48(8), pp. 683-690) so that the corresponding residue (V24) in CI8 was selected. Two more residues (N42, and T166) that are distantly positioned in the GAF domain and have been identified frequently from the active NifA mutants (Table 5) were selected for site-saturation mutagenesis using the LQ6 mutants as a template. Specifically, each mutant residue in the L6 mutant (V24, N43, and T166) was mutagenized using primers with degeneracy (e.g., NNK) to cover all 20 amino acids. The pooled libraries in E. coli were introduced into the nifA deleted CI8 in a manner similar to that described for the reporter plasmid. Colonies producing higher GFP levels than those from the L6 mutant were chosen for further analysis.

After isolation of the GFP expressing colonies from the agar plates, the nifH promoter induction with nifA variants was compared to the induction of the wild-type nifA and substituted residues were identified by sequencing. A total of 26 nifA variants that recover at least 10% of nifH promoter activity in the presence of ammonium was identified by flow cytometry. The mutant residues identified in more than one variant are listed in Table 3 with their occurrence number from the active NifA mutants. Out of three residues, only a substitution at Asn42 into Glu increased nifH expression up to 86% of that in the LQ6 mutant, suggesting that the combinations of mutations in the GAF domain and the Q-linker in NifA synergistically alleviated the ammonium effect on nitrogenase activity (FIG. 5).

TABLE 3
SNPs in ammonium insensitive NifA mutants in CI8

| | | Number of occurences from |
|---|---|---|
| Residue | Location | constituitively active mutants |
| Glu 8 | GAF domain | 2 |
| Glu 12 | GAF domain | 2 |
| Val 24 | GAF domain | 3 |
| Asn 42 | GAF domain | 5 |
| Ala 54 | GAF domain | 3 |
| Lys 90 | GAF domain | 2 |
| Ile 146 | GAF domain | 2 |
| Asp 159 | GAF domain | 3 |
| Thr 166 | GAF domain | 5 |
| Val 178 | Linker | 6 |
| Met 185 | Linker | 6 |
| Lys 194 | Linker | 2 |

### Development of a High-Throughput Screening System for Screening Ammonium Tolerant NifA in Azospirillum

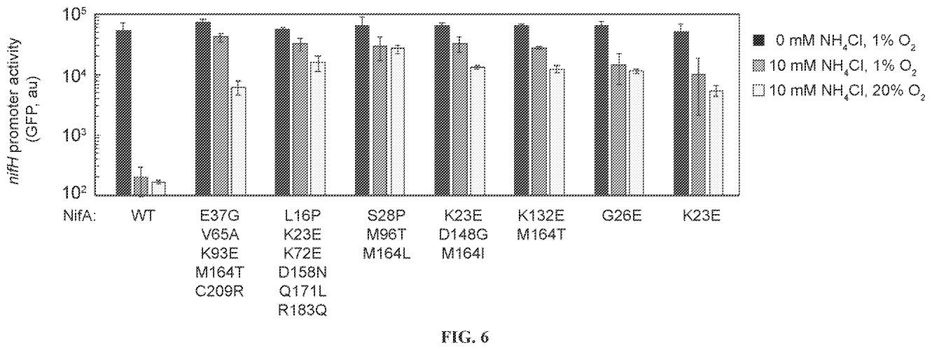

To identify NifA variants that remove ammonium repression, the genomic copy of nifA was deleted from Azospirillum strain CI1666 using the same method as for Paraburkholderia in Example 1. The ability of the system to recapitulate the native regulation of the nifH promoter with NifA complementation was assessed using the same method as described above for Paraburkholderia in Example 1. The nifH expression was decreased by two orders of magnitude with the addition of 10 mM ammonium, indicating that the system can select NifA mutants that do not repress the nif gene expression in the presence of ammonium while maintaining their activity regardless of ammonium availability (FIG. 6).

Identification of Ammonium Tolerant NifA in Azospirillum from Directed Evolution

To generate potentially ammonium resistant nifA mutants, the GAF domain and Q-linker of nifA were amplified from the genomic DNA of CI1666 by error-prone PCR and assembled with the plasmid based on a pBBR1 origin (FIG. 1b). Directed evolution approaches were applied to generate a nifA mutant library for CI1666 as described above. nifA variants were collated to a library size of 10⁶ recombinants in E. coli. The pooled nifA libraries were introduced into CI1666 in a manner similar to that described for the reporter plasmid (FIG. 1C). Triparental mating was used to transfer DNA from E. coli to Azospirillum. Conjugation was performed as described above. The mating mixtures were plated on SOB agar plates supplemented with 50 μg/ml ampicillin, 7.5 μg/ml gentamicin and incubated at 30° C. for 5 days under hypoxic conditions (1% oxygen). Derepression of the mf cluster was visualized by GFP expression and colonies showing induction of the nifH promoter arose with at a frequency of ˜10⁻⁴.

After isolation of the GFP expressing colonies from the agar plates, the nifH promoter induction with nifA variants to the one with the wild-type nifA were compared and substituted residues were identified by sequencing (FIG. 1c). More than 100 nifA variants that recover at least 10% of nifH activity in the presence of ammonium was identified by flow cytometry and identified residues are listed in Table 4 with the number of occurrences. Five NifA variants that recovered more than 20% of nifH activity in the presence of ammonium were selected for further analysis (FIG. 6). Among those, the nifH promoter activities in the NifA mutants with the residue E37G/V65A/K₉₃E/M164T/C209R and the residue L16P/K23E/K72E/D158N/Q171L/R183Q were more than half of the promoter activity with the wild-type NifA.

To compare oxygen tolerance of these NifA variants, the nifH promoter activity was analyzed in the presence of atmospheric oxygen (20% oxygen) and ammonium (FIG. 6). Single colonies were inoculated into 0.5 ml SOB medium supplemented with 7.5 μg/ml gentamicin in 96-deep-well plates and incubated overnight at 30° C. and 900 r.p.m. Aliquots (4 μl) of the overnight cultures were diluted in 200 μl Azospirillum minimal medium containing 10 mM ammonium chloride and 7.5 μg/ml gentamicin in 96-well plates, and incubated for 24 hr at 30° C. and 900 r.p.m. Aliquots (10 μl) of these cultures were then diluted in 150 μl PBS with 2 mg/ml kanamycin for flow cytometry analysis. Cultures with fluorescence proteins were analyzed by flow cytometry as described above. The NifA mutant with the residue S28P/M96T/M164L reaches the highest activity in the presence of oxygen and ammonium and has 162-fold higher activity than the wild-type NifA.

TABLE 4
SNPs in ammonium insensitive NifA mutants in CI1666

| | | Number of occurrences from |
|---|---|---|
| Residue | Location | constituitively active NifA mutants |
| Pro 2 | GAF domain | 5 |
| Ser 11 | GAF domain | 4 |
| Leu 16 | GAF domain | 5 |
| Lys 23 | GAF domain | 11 |
| Ile 24 | GAF domain | 3 |
| Gly 26 | GAF domain | 3 |
| Ser 27 | GAF domain | 9 |
| Ser 28 | GAF domain | 7 |
| Asp 30 | GAF domain | 4 |
| Val 65 | GAF domain | 5 |
| Ile 87 | GAF domain | 4 |
| Lys 93 | GAF domain | 4 |
| Met 96 | GAF domain | 4 |
| Asn 102 | GAF domain | 3 |
| Leu 108 | GAF domain | 3 |
| Asp 120 | GAF domain | 5 |
| Glu 121 | GAF domain | 5 |
| Gln 122 | GAF domain | 6 |
| Ala 124 | GAF domain | 3 |
| Lys 132 | GAF domain | 5 |
| Asp 144 | GAF domain | 3 |
| Val 159 | GAF domain | 3 |
| Thr 163 | GAF domain | 3 |
| Met 164 | GAF domain | 11 |
| Val 179 | Linker | 3 |
| Arg 183 | Linker | 5 |
| Phe 185 | Linker | 3 |
| Met 186 | Linker | 6 |
| Met 187 | Linker | 8 |
| Phe 191 | Linker | 4 |
| Gln 194 | Linker | 3 |
| Lys 195 | Linker | 4 |
| Thr 210 | AAA domain | 3 |

### Example 3—Rational Design Approach to Develop Constitutively Active NifA in Paraburkholderia tropica, Paraburkholderia xenovorans, Paraburkholderia phymatum, Herbaspirillum seropedicae, Herbaspirillum frisingense, and Azospirillum lipoferum

In this example, rational design approaches were used to remove self-inhibition of nifA and increase ammonium production in Paraburkholderia tropica (strain CI8), Herbaspirillum seropedeciae (strain CI3000), Herbaspirillum frisingense (strain CI1663/LMG 23164), and Azospirillum lipoferum (strain CI1666/LMG 13128).

The residues in the GAF domain and the Q-linker of NifA responsible for ammonium tolerance identified in this study are highly conserved within the genus of Paraburkholderia and Azospirillum that contain the nif cluster. In the case of Paraburkholderia, multisequence alignments using the N-terminal domains from six Paraburkholderia species (P. tropica CI8 (SEQ ID NO: 14), P. xonovorans (SEQ ID NO: 37), P. aromaticivorans (SEQ ID NO: 38), P. kururiensis (SEQ ID NO: 39), P. phymatum (SEQ ID NO: 40), and P. phenoliruptrix (SEQ ID NO: 41)) were constructed and confirmed that the residues identified from the high-throughput screening and functional analyses are highly conserved across Paraburkholderia species (FIG. 7A). Highly conserved residues that are responsible for constitutively active NifA and are present in CI8 include V24, R34, A54, K90, D108, P116, T166, V178, M185, Q186, K194, and 1196 and are denoted with a filled circle in FIG. 7A. In the case of Azospirillum, multisequence alignments using the N-terminal domains from 13 Azospirillum species (A. lipoferum CI1666 (SEQ ID NO: 15), A. oleiclasticum (SEQ ID NO: 42), A. halopraeferens (SEQ ID NO: 43), A. thermophilum (SEQ ID NO: 44), A. formosense (SEQ ID NO: 45), A. brasilense (SEQ ID NO: 46), A. baldaniorum (SEQ ID NO: 47), A. doebereinerae (SEQ ID NO: 48), A. oryzae (SEQ ID NO: 49), A. melinis (SEQ ID NO: 50), A. palustre (SEQ ID NO: 51), A. ramasamyi (SEQ ID NO: 52), and A. humicireducens (SEQ ID NO: 53)) were constructed. The residues that make NifA tolerant to ammonium, allowing the expression of the nif cluster in the presence of ammonium, are extremely well conserved across Azospirillum as the NifA proteins from Azospirillum species are more closely related compared to Paraburkhoderia species (FIG. 7B). Highly conserved residues that are responsible for constitutively active NifA and are present in CI1666 include P2, S12, L16, K23, 124, G26, S27, S28, D30, E37, V65, I87, K93, M96, N102, L108, D120, E121, Q122, A124, K132, D148, D158, V159, T163, M164, Q171, V179, R183, F185, M186, M187, F191, Q194, K195, C209, and T210 and are denoted with a filled circle in FIG. 7B.

Residues were identified from each of the Paraburkholderia (SEQ ID NO:14) and Azospirillum (SEQ ID NO: 15) genera that co-occur across the two represented two proteobacteria (i.e. alpha- and beta-proteobacteria). One residue from the GAF domain (K93) and three residues from the Q-linker domain were identified (FIG. 7C), including V182, M189, K198. A high level of conservation of these residues increases the likelihood that substitutions, mutations, or deletions of these conserved residues in NifA will render nitrogenase expression tolerant to ammonium repression across diverse species ranging from alpha-proteobacteria to beta-proteobacteria. Thus, the high-throughput screening system described here is widely applicable in alpha- and beta-proteobacteria for identifying mutations in a master regulator of nitrogen fixation that overcome ammonium repression, and the nifA mutants identified here can be adapted to remove ammonium repression in nitrogen fixing alpha- and beta-proteobacteria that lack the nifL regulation. Mutations identified with this screening system can be substituted, mutated, or deleted.

Engineering Paraburkholderia tropica for Derepression of Nitrogen Fixation and Increased Ammonium Production

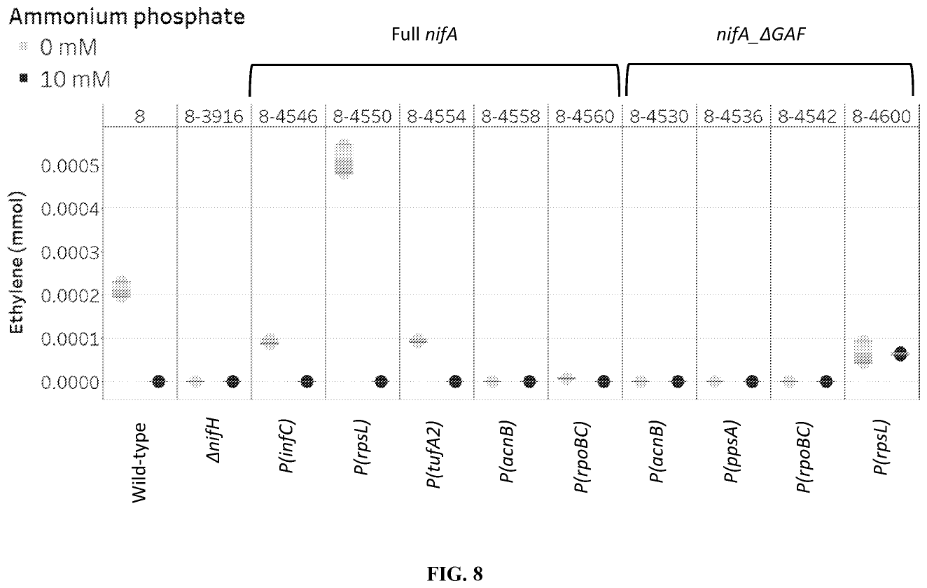

To remove the nitrogen-dependent transcriptional regulation of the nifA gene, strong constitutive promoters of Paraburkholderia tropica were characterized by selecting promoters of highly expressed genes as measured by RNAseq analysis (PCT Publication No.: WO/2019/084059). Selected promoters were cloned upstream of the genomic copy of the full length or GAF-domain deleted nifA (ΔGAF-nifA) and the promoter of rpsL gene was shown to drive highest derepression of nitrogenase activity and ammonium production (FIG. 8).

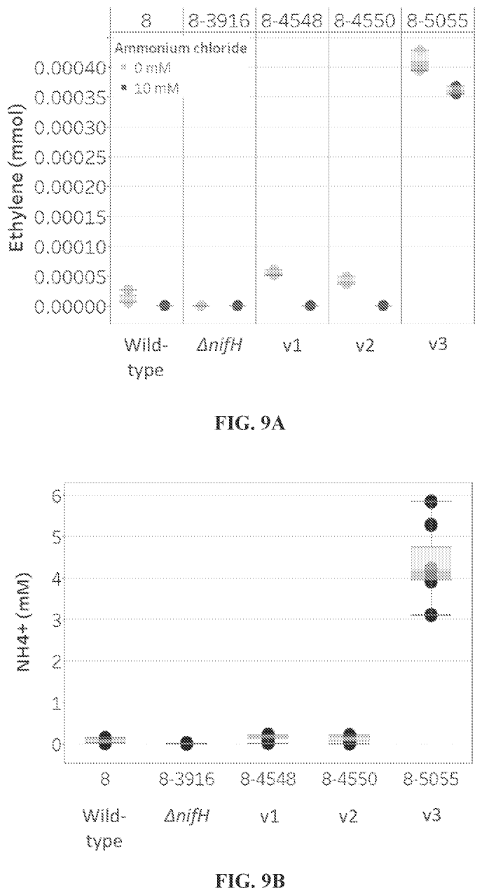

Next, different strategies were tested for inserting the constitutive promoter upstream of the genomic copy of nifA gene. In one version, the native promoter sequence of the genomic nifA was fully maintained and the constitutive promoter was inserted immediately upstream of the start codon. In another version, 180 bp region immediately upstream of the nifA coding sequence that presumably covers all the promoter sequence was replaced by the constitutive promoter. Lastly, in another version, 100 bp upstream of the start codon of the nifA gene was replaced by the constitutive promoter of choice, to see whether the native promoter sequences further improved derepression of the nifA variant in conjunction with the constitutive promoter. An ARA and ammonium measurement assay (AMM) showed that keeping the native promoter intact upstream of the constitutive promoter further improved derepression (FIGS. 9A and 9B). Therefore, all other variants were cloned by keeping the native upstream sequences intact.

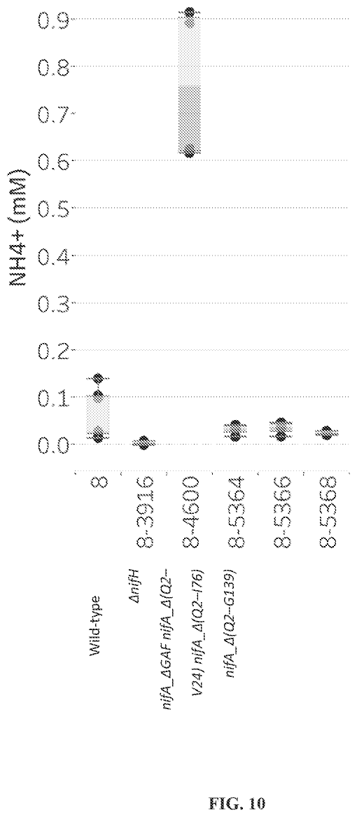

Though deletion of GAF domain removes self-inhibition of nifA, it also decreases NifA protein stability, as evidenced by decreased nitrogen fixation activity in the absence of fixed nitrogen compared to the wild type nifA (8-4550 compared to 8-4600 in FIG. 8). To alleviate this problem, shorter truncations were introduced to the N-terminal end of P. tropica (8) by deleting the N-terminus of nifA from Q2 to V24, from Q2 to 176, and from Q2 to G139. However none of the smaller truncations showed strong derepression of nitrogen fixation and only ΔGAF-nifA (strain 8-4600) modification showed significant accumulation of ammonium (FIG. 10).

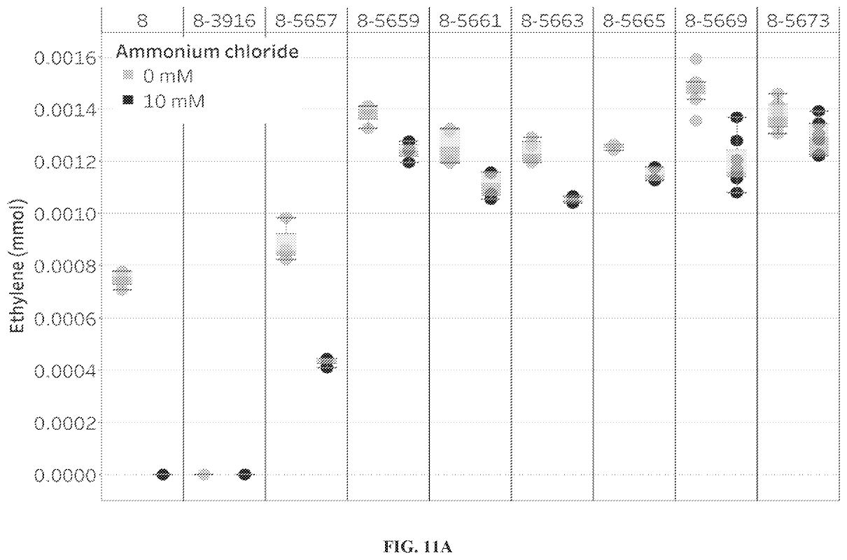

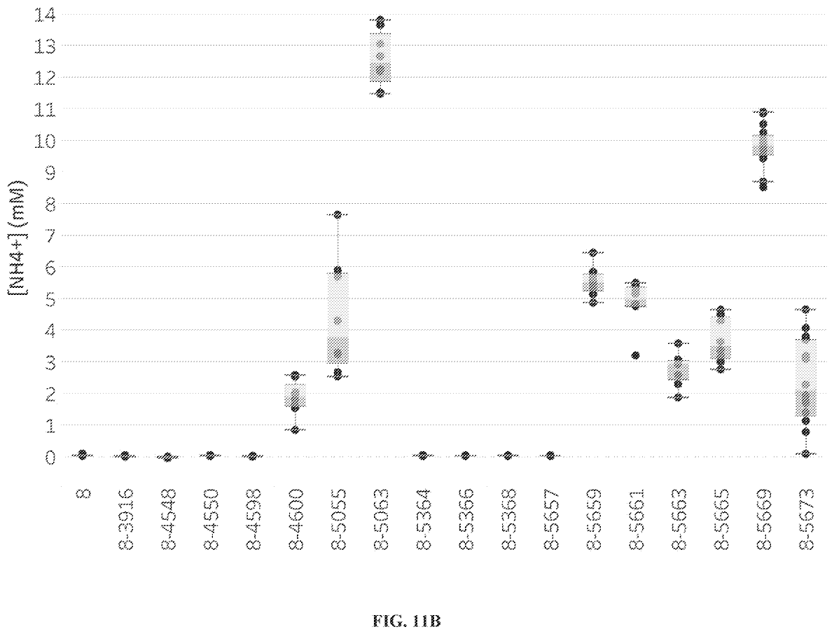

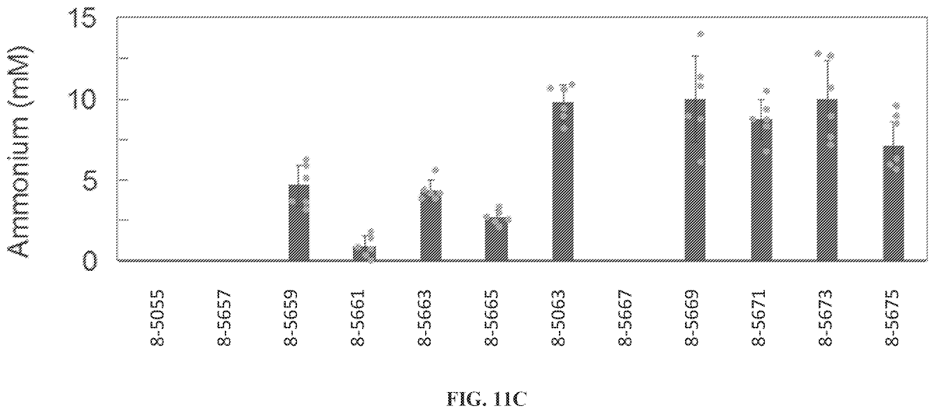

Next, the nifA variants were cloned in the genomic context to see if they lead to derepression of nitrogen fixation under rich nitrogen conditions. The 5 nifA variants [D108E, D159T, T166A, M185T], [N42D, D121A, T166A], [N42S, V178A], [Q186R, I196V], [G7D, R34Q, M93V, P116H, V178M] were individually cloned under the PrpsL promoter and inserted in place of the genomic copy of nifA. ARA data showed that some of the newly identified variants (8-5659, 8-5661, 8-5663, 8-5665) showed higher nitrogenase activity than ΔGAF-nifA (FIG. 11A). The ammonium measurements were done both with the Megazyme and OPA assays (FIGS. 11B-11C).

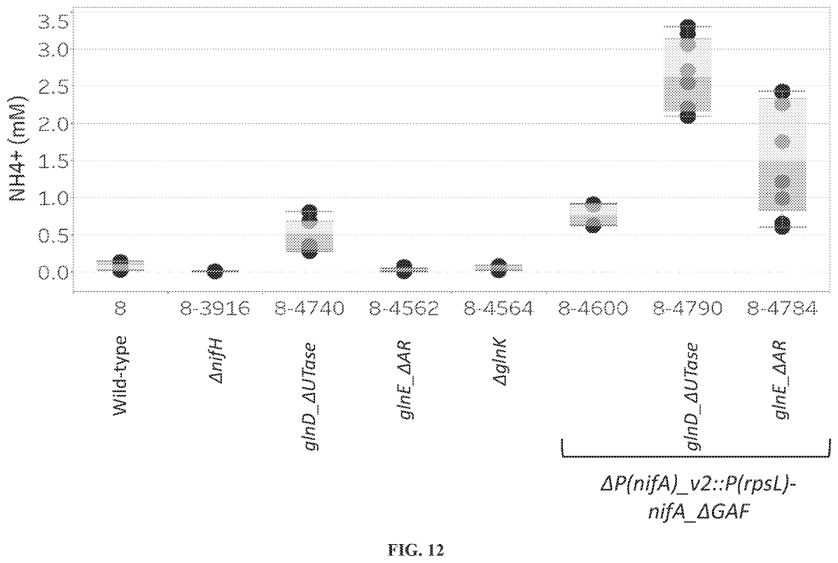

To increase accumulation of ammonium further, additional genomic edits were introduced to further increase ammonium production including deleting of GlnE's adenylyl removal domain (glnE_ΔAR) or the uridyl transferase domain of GlnD (glnD_ΔUTase) to down-regulate assimilation of ammonium through glutamine synthetase activity. These modifications were introduced individually and also in combination with the ΔGAF-nifA modification. Additionally, a ΔglnK modification was introduced as the deletion of glnK gene was previously shown to enhance the activity of NifA, when its GAF domain was deleted. Though both glnE_ΔAR and glnD_ΔUTase modifications further increased ammonia production when stacked on top of ΔGAF-nifA modification, deleting glnK did not increase ammonium production further (FIG. 12).

### Engineering Herbaspirillum and Azospirillum Species for Derepression of Nitrogen Fixation and Increased Ammonium Production

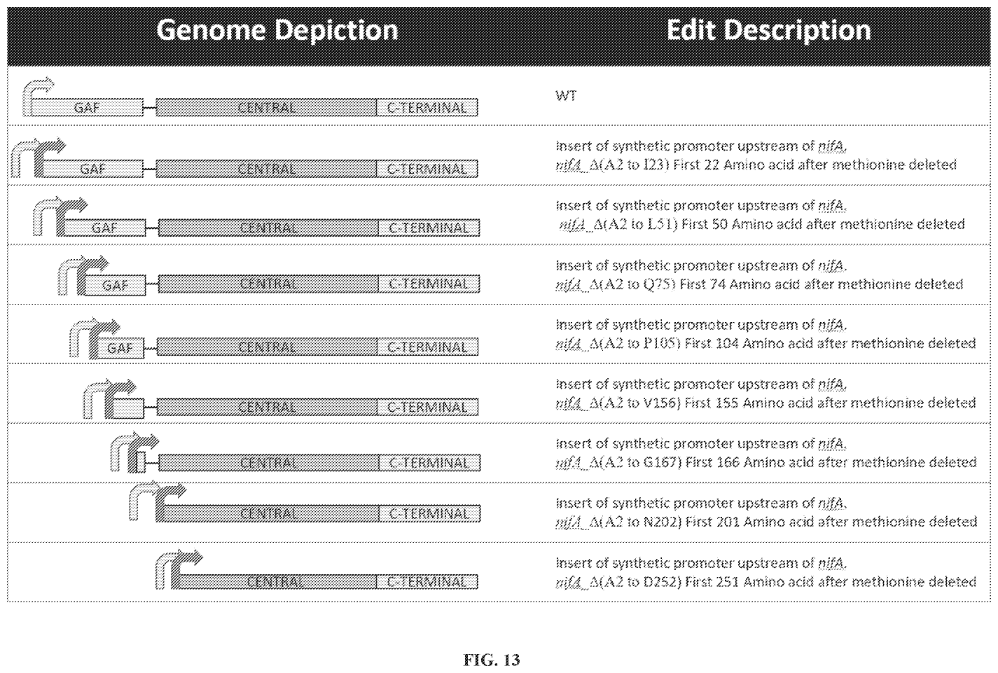

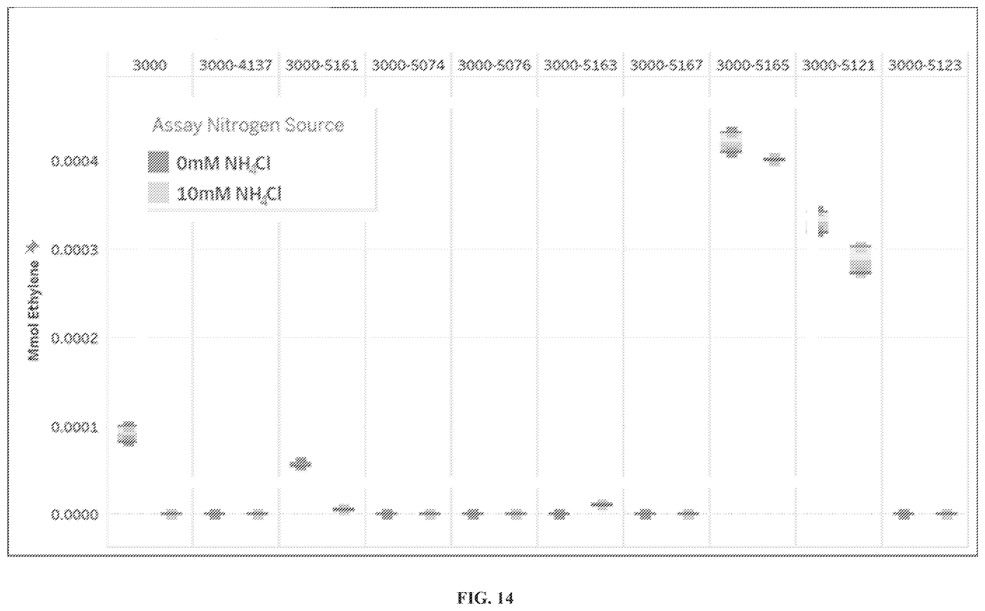

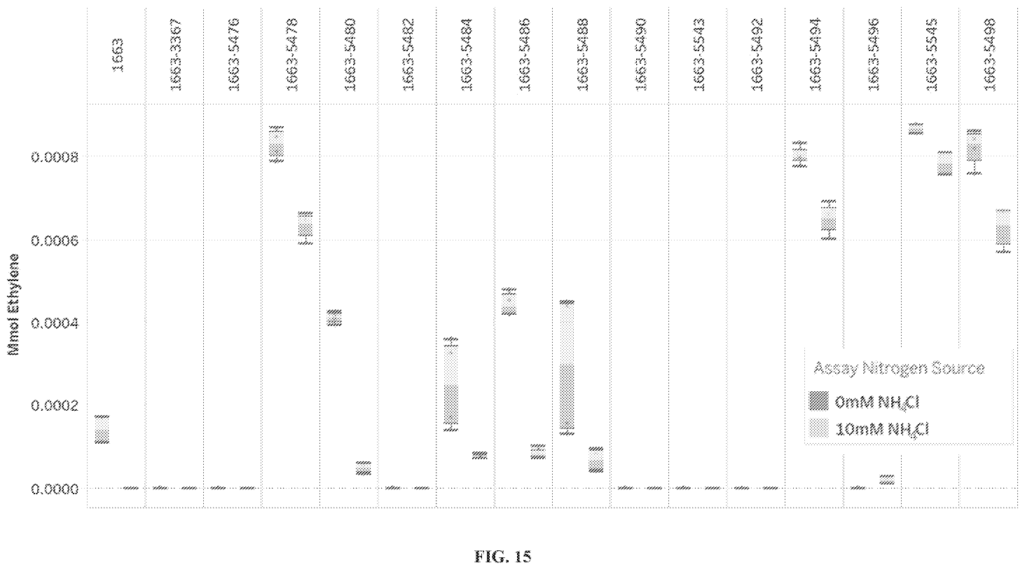

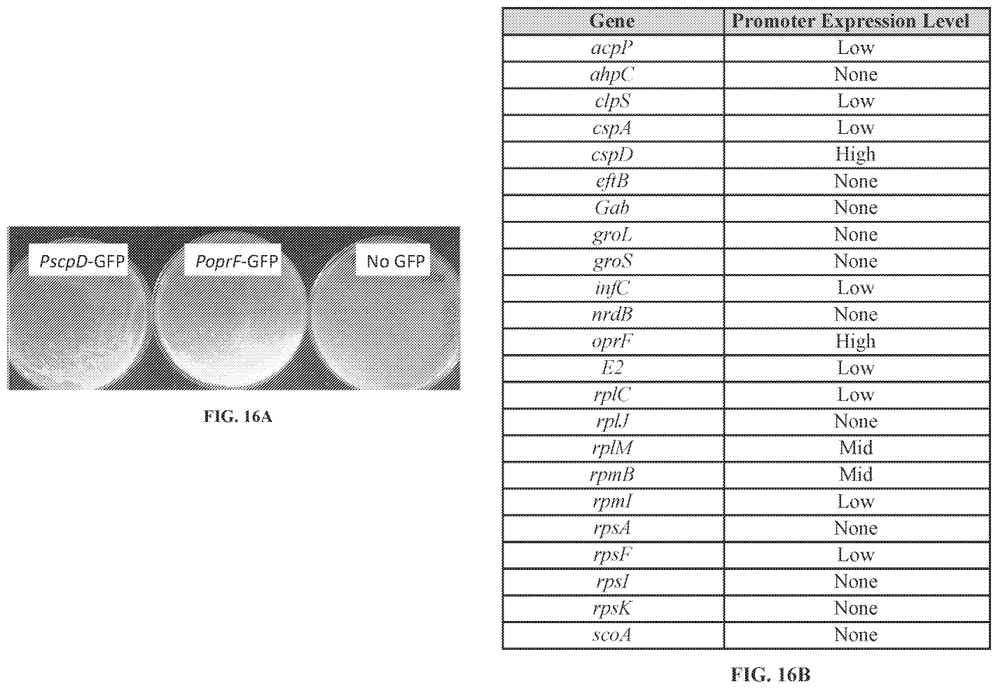

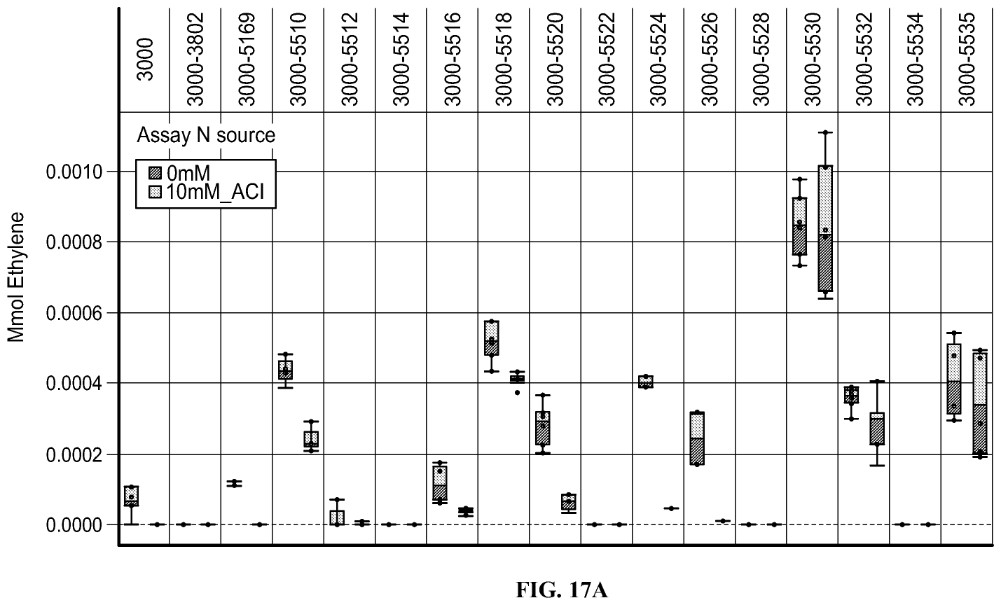

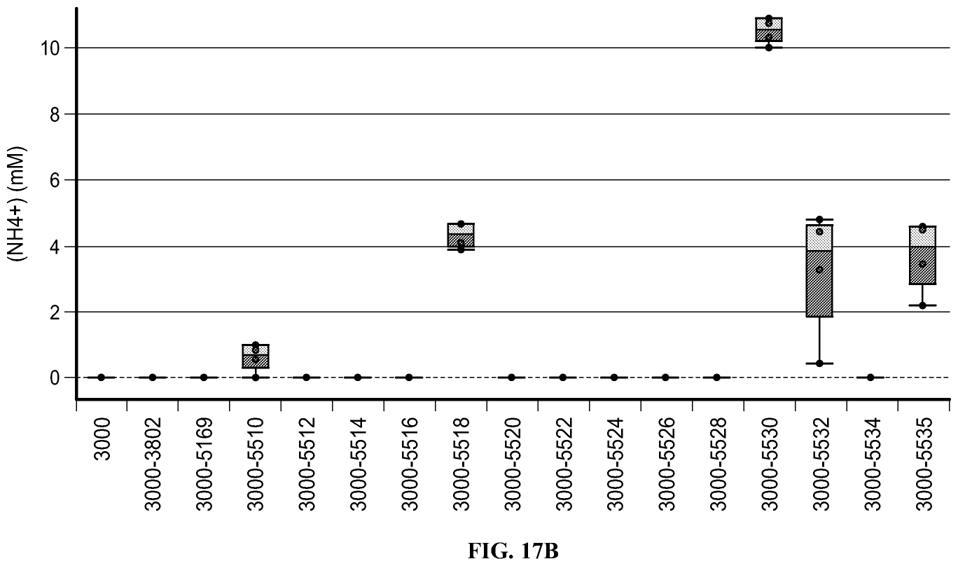

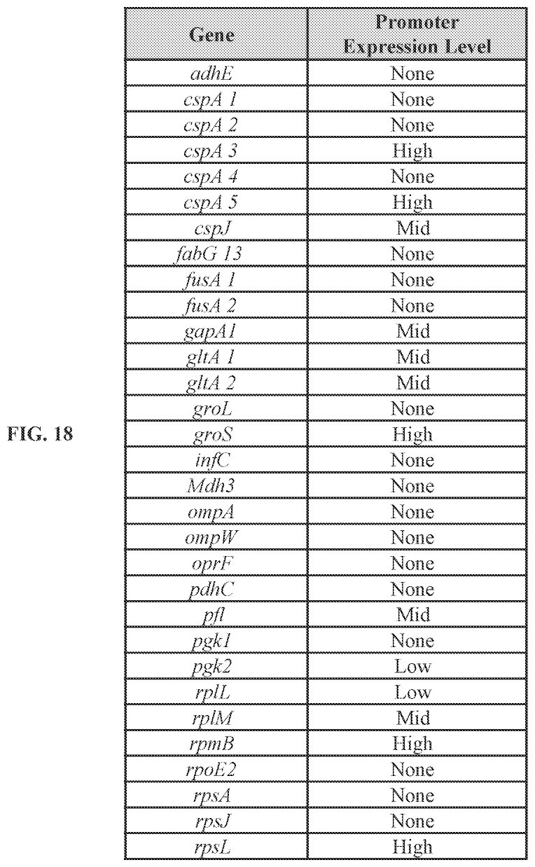

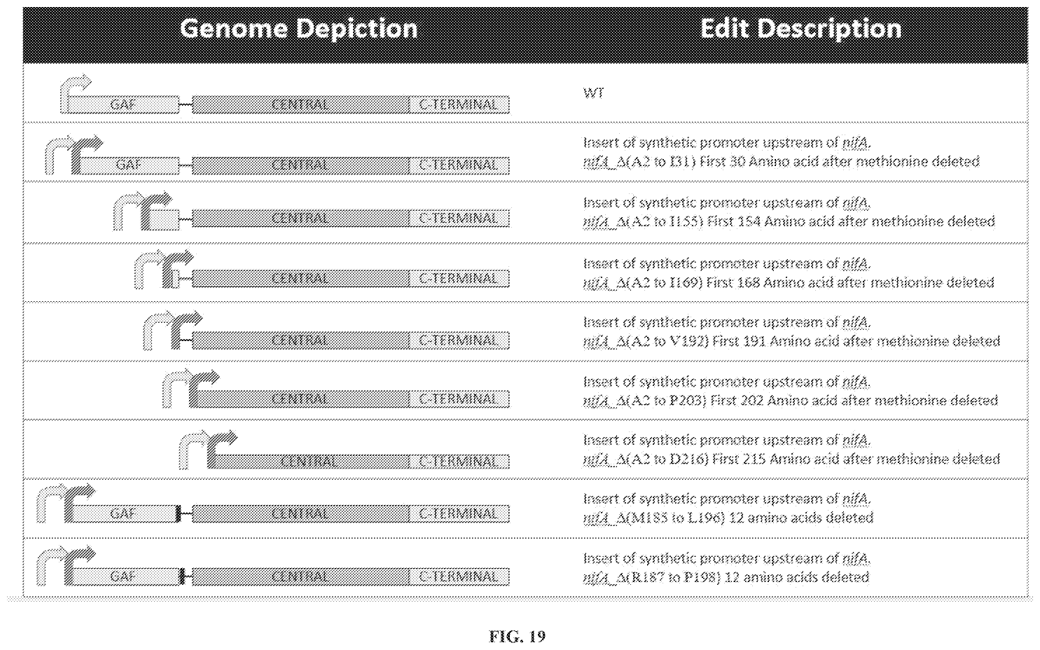

In another study, N-terminal deletions to the nufA genes of H. seropedicae (3000) (FIG. 13), H. frisingense (1663), and A. lipoferum (1666) were introduced. In both Herbaspirillum species, deletion of either the full GAF domain (ΔA2-N202) or most of the GAF domain (ΔA2-G167) showed the highest level of derepression (FIGS. 14-15). The primary screen for the activity of various nifA truncations were done under a strong synthetic promoter P(lil). Subsequently, the top performing nifA variants were cloned under high expressing native promoters and inserted into the genome while replacing the wild-type genomic copy of the nufA gene. To identify high expressing native promoters, highly expressed genes from an RNAseq dataset of diverse genera of strains was analyzed. The promoter regions of the homologs of these genes (200-400 bp sequences upstream of the start codons) in H. seropedicae (3000) and A. lipoferum (1666) were cloned upstream of the green fluorescent reporter gene on a plasmid. Strong promoters were characterized based on the GFP signal (FIG. 16 and FIG. 18). For Herbaspirillum, cspD and oprF promoters were identified as high expressing constitutive promoters, whereas for Azospirillum cspA and rpmB showed the highest expression of GFP. Some of these promoters driving the expression of nufA variants were used individually and also in combination with glnD_ΔUTase modification (FIG. 17). The 3000-5530 strain (P(cspD)_nifA_ΔA2-G167 glnD_ΔUTase) showed the highest derepression of nitrogen fixation and ammonium production. In addition to N-terminal truncation, a mutation changing the glycine amino acid (the 25th amino-acid) to glutamate (G25E) in Herbaspirillum strains was introduced; however, the G25E mutation did not lead to derepressed NifA activity in 3000 (strain 3000-5169, FIG. 17) Engineered strains of Herbaspirillum are described in tables 5-7.

TABLE 5
H. seropedicae (3000) strains with nested N-terminal
deletions of nifA under strong synthetic promoter (Plil).

| | | ARA | ARA | |
|---|---|---|---|---|
| | | Ethylene | Ethylene | |
| | | in 0 mM | in 10 mM | |
| Strain | | NH4Cl | NH4C1 | [NH4+] |
| ID | Genotype | (mm/OD) | (mm/OD) | (mM) |
| CI3000 | WT | 0.109E-13- | 0 | 0.111 |
| | | 0.12E-13 | | |
| 3000-4137 | ΔnifHDK | 0 | 0 | 0.003 |
| 3000-5161 | P(lil)-nifA_Δ(A2 to 123) | 0.070E-13 | 0.0045E-13- | #N/A |
| | | | 0.005E-13 | |
| 3000-5074 | P(lil)-nifA_Δ(A2 to L51) | 0 | 0 | #N/A |
| 3000-5076 | P(lil)-nifA_Δ(A2 to Q75) | 0 | 0 | #N/A |
| 3000-5163 | P(lil)-nifA_Δ(A2 to p105) | 0 | 0.0089E-13- | #N/A |
| | | | 0.009E-13 | |
| 3000-5167 | P(lil)-nifA_Δ(A2 to V156) | 0 | 0 | #N/A |
| 3000-5165 | P(lil)-nifA_Δ(A2 to G167) | 0.5E-13- | 0.49E-14- | 0.047 |
| | | 0.693E-13 | 0.727E-13 | |
| 3000-5121 | P(lil)-nifA_Δ(A2 to N202) | 0.355E-13- | 0.308E-13- | #N/A |
| | | 0.36E-13 | 0.31E-13 | |
| 3000-5123 | P(lil)-nifA_Δ(A2 to D252) | 0 | 0 | #N/A |

TABLE 6
H. seropedicae (3000) strains with ΔGAF-nifA under strong native promoters.

| | | ARA | ARA | |
|---|---|---|---|---|
| | | Ethylene | Ethylene | |
| | | in 0 mM | in 10 mM | |
| Strain | | NH4C1 | NH4Cl | [NH4+] |
| ID | Genotype | (mm/OD) | (mm/OD) | (mM) |
| CI3000 | WT | 0.109E−13- | 0 | 0.007-0.111 |
| | | 7.3E−15 | | |
| 3000-3802 | ΔnifH | 0 | 0 | 0.005-0.410 |
| 3000-5169 | P(lil)-nifA_G25E | 0.118E−13- | 0 | 0.0008-0.011 |
| | | 0.13xE−13 | | |
| 3000-5510 | P(cspD)::nifA-ΔA2-N202 | 0.442E−13- | 0.22E−13- | 0.041-0.59 |
| | | 0.52E−13 | 0.224E−13 | |
| 3000-5512 | P(oprF)_nifA_ΔA2-N202 | 0.003E−13- | 0.007E−13- | 0.003-1.431 |
| | | 5.3E−15 | 0.0075E−13 | |
| 3000-5514 | P(rplM)_nifA_ΔA2-N202 | 0 | 0 | 2E−3 |
| 3000-5516 | P(rpmB)_nifA_ΔA2-N202 | 0.093E−13 | 0.030E−13 | 5E−3 |
| 3000-5518 | P(cspD)_nifA_ΔA2-G167 | 0.596E−13- | 0.038E−13- | 3.290-4.3 |
| | | 0.064E−13 | 0.387E−13 | |
| 3000-5520 | P(oprF)_nifA_ΔA2-G167 | 0.261E−13- | 0.066E−13 | 0.004-0.033 |
| | | 0.31E−13 | | |
| 3000-5522 | P(rplM)_nifA_ΔA2-G167 | 0 | 0 | 0.006 |
| 3000-5524 | P(rpmB)_nifA_ΔA2-G167 | 0.306E−13- | 0.036E−13- | 0.005 |
| | | 0.31E−14 | 0.037E−13 | |
| 3000-5526 | P(rpsF)_nifA_ΔA2-G167 | 0.24E−13- | 0.0055E−13- | 0.02 |
| | | 0.244E−13 | 0.006E−13 | |
| 3000-5528 | P(rplM)_nifA_AA2-N202 | 0 | 0 | 0.002 |
| | glnD_ΔUTase | | | |
| 3000-5530 | P(cspD)_nifA_ΔA2-G167 | 2.3E−13- | 1.3E−13- | 1.101-10.5 |
| | glnD_ΔUTase | 2.508E−13 | 1.631E−13 | |
| 3000-5532 | P(oprF)_nifA_ΔA2-G167 | 0.68E−13- | 0.728E−13- | 0.039-3.2 |
| | glnD_ΔUTase | 0.698E−13 | 0.73E−14 | |
| 3000-5534 | P(rplM)_nifA_ΔA2-G167 | 0 | 0 | 0.02 |
| | glnD_ΔUTase | | | |
| 3000-5535 | P(rpmB)_nifA_ΔA2-G167 | 0.55E−13- | 0.55E−13- | 0.040-3.7 |
| | glnD_ΔUTase | 0.684E−13 | 0.553E−13 | |

TABLE 7
H. frisingense (1663) strains with ΔGAF-nifA driven by strong native promoters.

| | | ARA | ARA | |
|---|---|---|---|---|
| | | Ethylene | Ethylene | |
| | | in 0 mM | in 10 mM | |
| Strain | | NH4C1 | NH4Cl | [NH4+] |
| ID | Genotype | (mm/OD) | (mm/OD) | (mM) |
| CI1663 | WT | 0.11E−13- | 0 | 0 |
| | | 0.131E−13 | | |
| 1663-3367 | ΔnifH | 0 | 0 | 0 |
| 1663-5476 | P(cspA)::nifA-ΔA2-N202 | 0 | 0 | 0 |
| 1663-5478 | P(cspD-1)_nifAΔA2-N202 | 0.739E−13- | 0.46E−13- | 0.241 |
| | | 0.074E014 | 0.465E−13 | |
| 1663-5480 | P(cspD-2)_nifA_ΔA2-N202 | 0.034E−13- | 0.028E−13- | 0 |
| | | 0.341E−13 | 0.029E−15 | |
| 1663-5482 | P(infC)::nifA_ΔA2-N202 | 0 | 0 | 0 |
| 1663-5484 | P(ompA)::nifA_ΔA2-N202 | 0.2E−13- | 0.46E−13- | 0 |
| | | 0.376E−13 | 0.048E−13 | |
| 1663-5486 | P(rpmB)::nifA_ΔA2-N202 | 0.223E−13- | 0.043E−13- | 0 |
| | | 0.38E−13 | 0.048E−13 | |
| 1663-5488 | P(rpsF)::nifA_ΔA2-N202 | 0-2.2E−14 | 0-4.3E−15 | 0 |
| 1663-5490 | P(cspA)::nifA_ΔA2-G167 | 0 | 0 | 0 |
| 1663-5492 | P(infC)::nifA_ΔA2-G167 | 0 | 0 | 0.418 |
| 1663-5494 | P(ompA)::nifA_ΔA2-G167 | 0.690E−13 | 0.48E−13 | 0 |
| 1663-5496 | P(rpIM)::nifA_ΔA2-G167 | 0 | 0.011E−15 | 0.299 |
| 1663-5498 | P(rpsF)::nifA_ΔA2-G167 | 0.733E−13 | 0.436E−13- | 0 |
| | | | 0.44E−13 | |
| 1663-5543 | P(cspD-2)::nifA_ΔA2-G167 | 0 | 0 | 0.164 |
| 1663-5545 | P(rpmB)::nifA_ΔA2-G167 | 0.376E−13- | 0.048E−13- | 0 |
| | | 0.87E−14 | 0.74E−14 | |

### Example 4—Identifying Universal and Causal SNPs in the GAF Domain of NifA that Removes Self-Inhibition

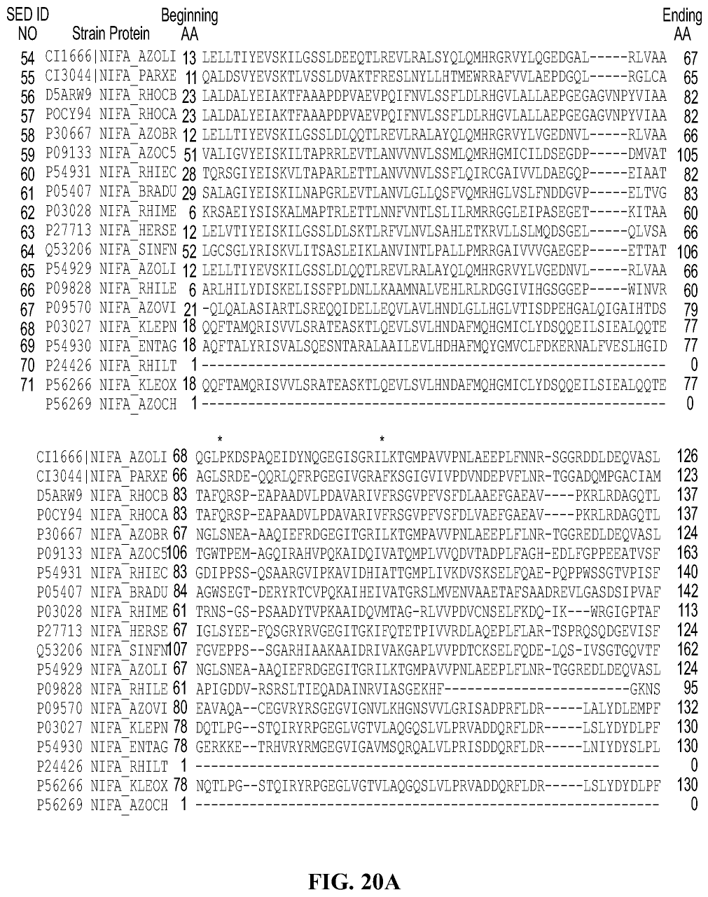

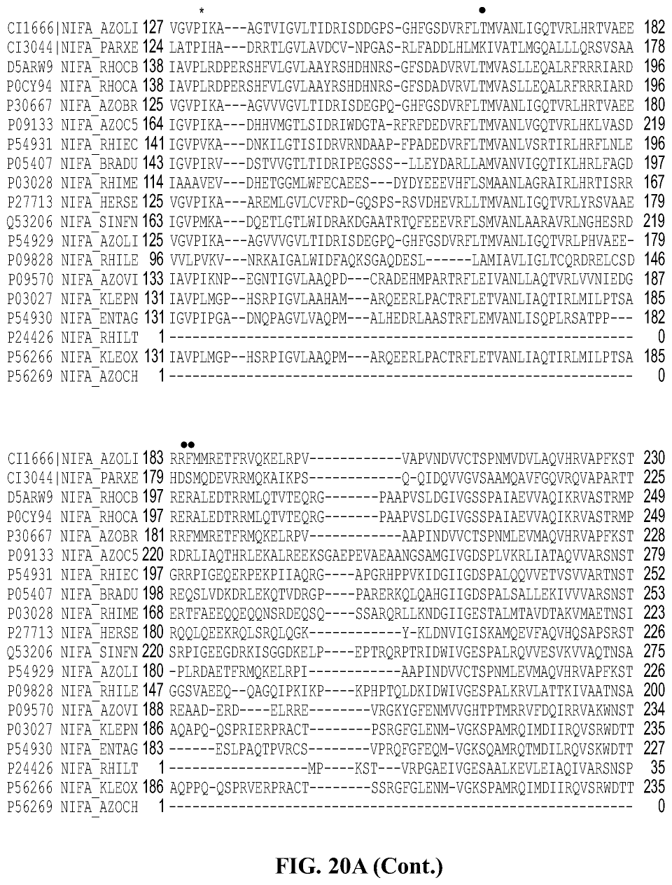

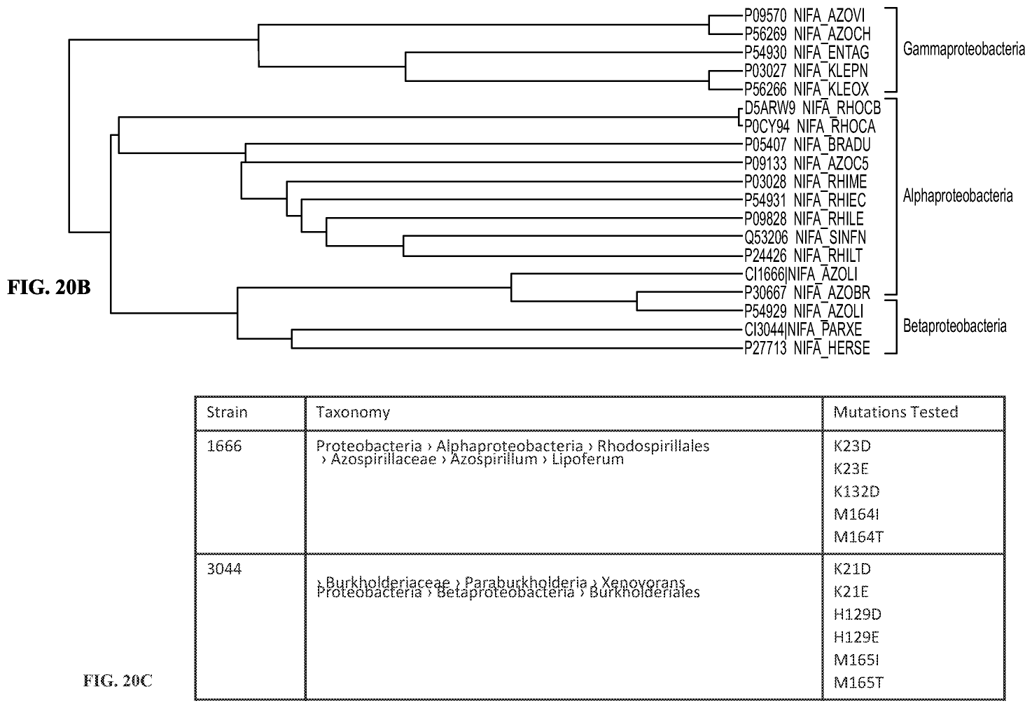

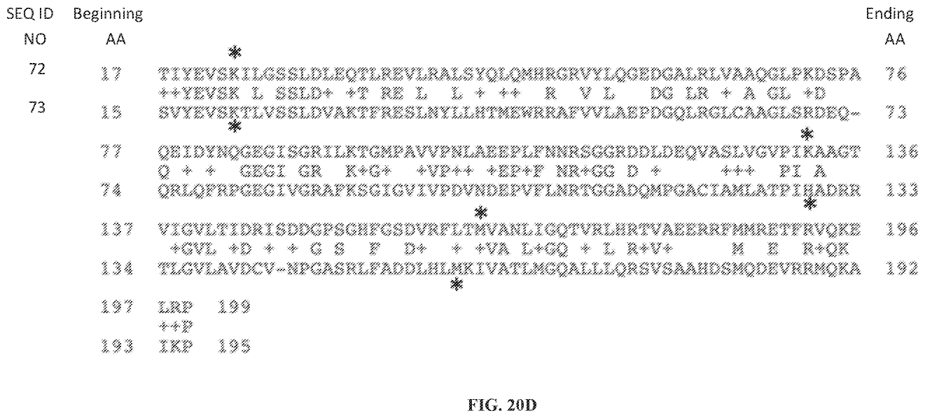

The SNPs characterized in strain CI1666 in Example 3 were used to identify mutations with the most significant impact on NifA activity that were also conserved across different classes of bacteria. Frequently isolated mutations showed that most of the lysine residues in the GAF domain were frequently mutated into glutamate residues, such as K23E, K72E, K93E, and K132E (FIG. 6). Methionine residues M164, M186, and M187 also were frequently mutated in the CI1666 strain. Multiple sequence alignment of previously reviewed NifA proteins from the Uniprot database (SEQ ID Nos: 54-71) showed that K23 was conserved in both alpha and beta proteobacteria, whereas M164 was only conserved in alpha-proteobacteria (FIGS. 20A-20B). Therefore, the well conserved K23 and M164 residues as well as the K132 residue that was frequently mutated in the mutational analysis were analyzed. The lysine residues were mutated either to glutamate or aspartate, to test whether converting lysine to any acidic residue would impact NifA activity. The mutated methionine residues showed no strong mutational preference, as long as they were mutated to another amino acid such as isoleucine, threonine, or leucine. For introduced mutations of Azospirillum and Paraburkholderia strains, see FIG. 20C and the sequence alignment (SEQ ID Nos: 72 and 73) in FIG. 20D.

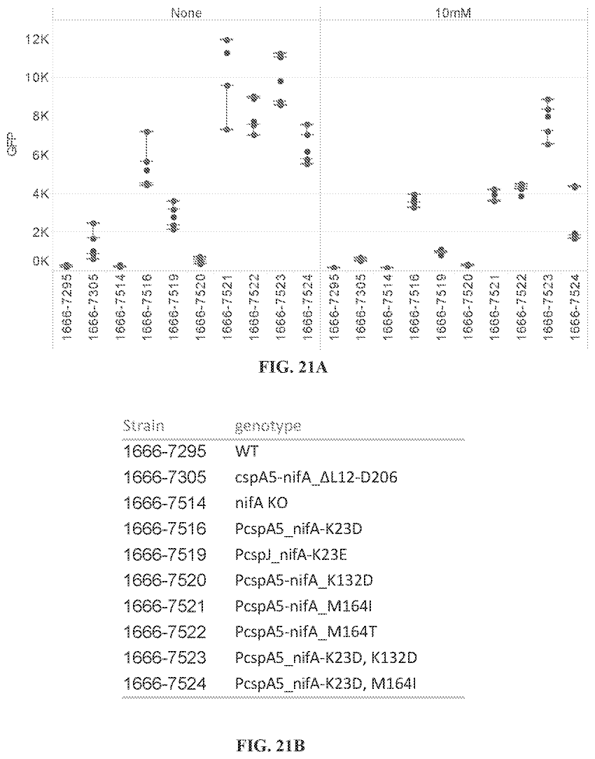

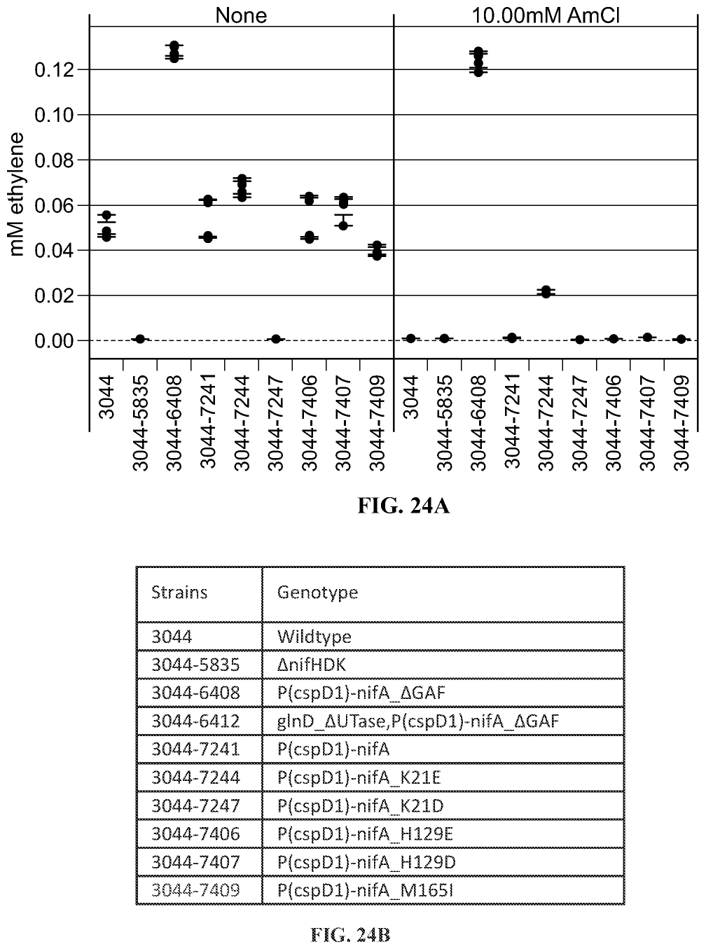

The transcriptional activity of NifA in various mutants was assessed via a nifH promoter-driven GFP gene (PnifH-GFP), both in CI1666 and CI3044 (FIGS. 21 and 24). In CI1666, K23D, K23E, M164I, and M164T mutations all showed NifA activity under rich nitrogen conditions, while K132 mutation did not show any NifA activity. In the CI3044 background, K21E showed the strongest activity, while K21D and M164I/T mutations showed only marginal activity.

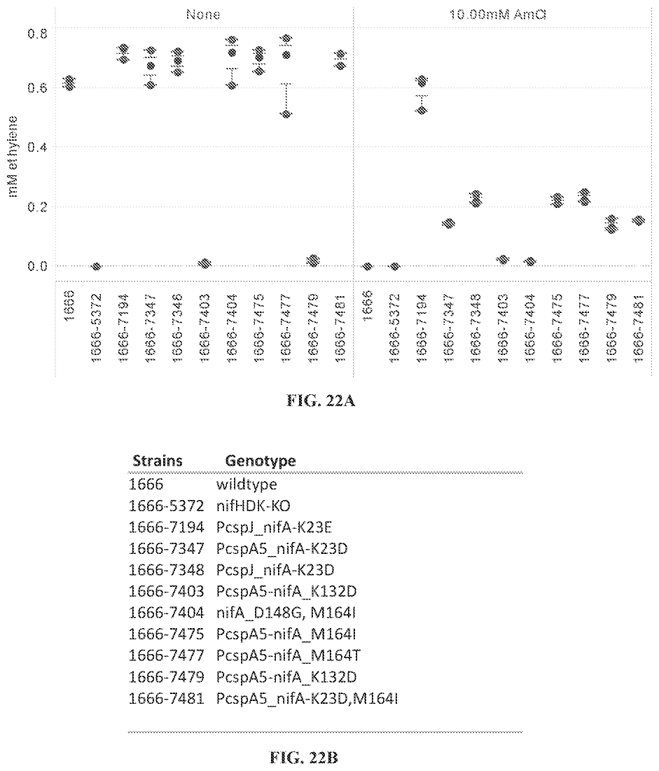

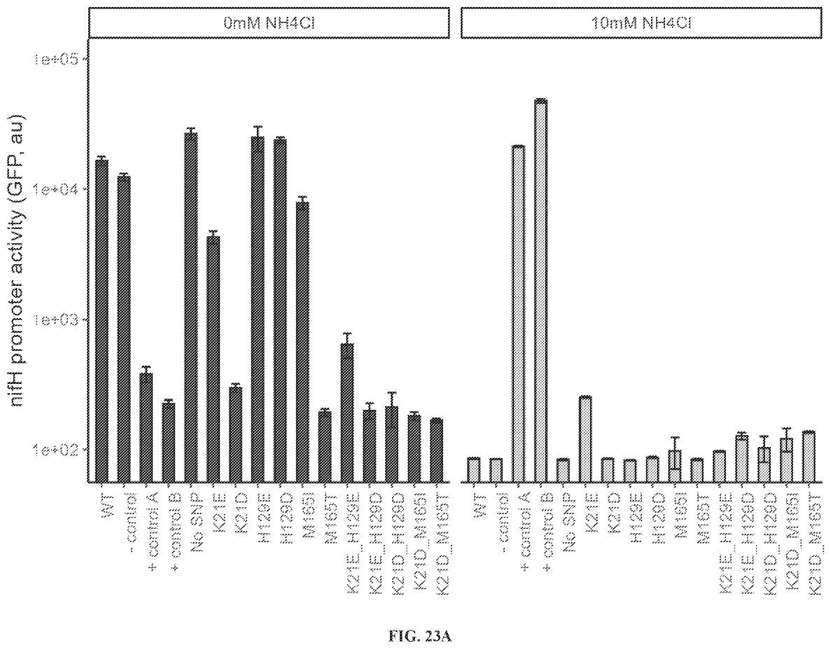

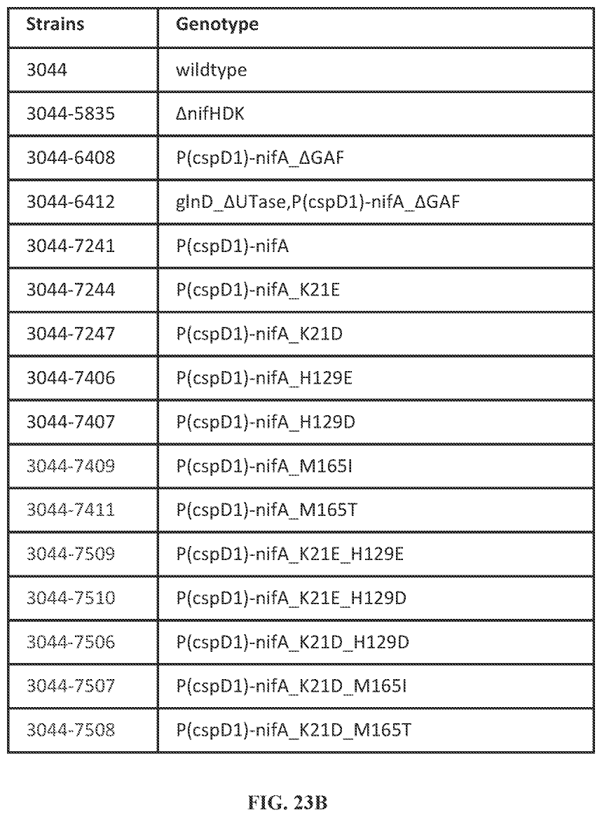

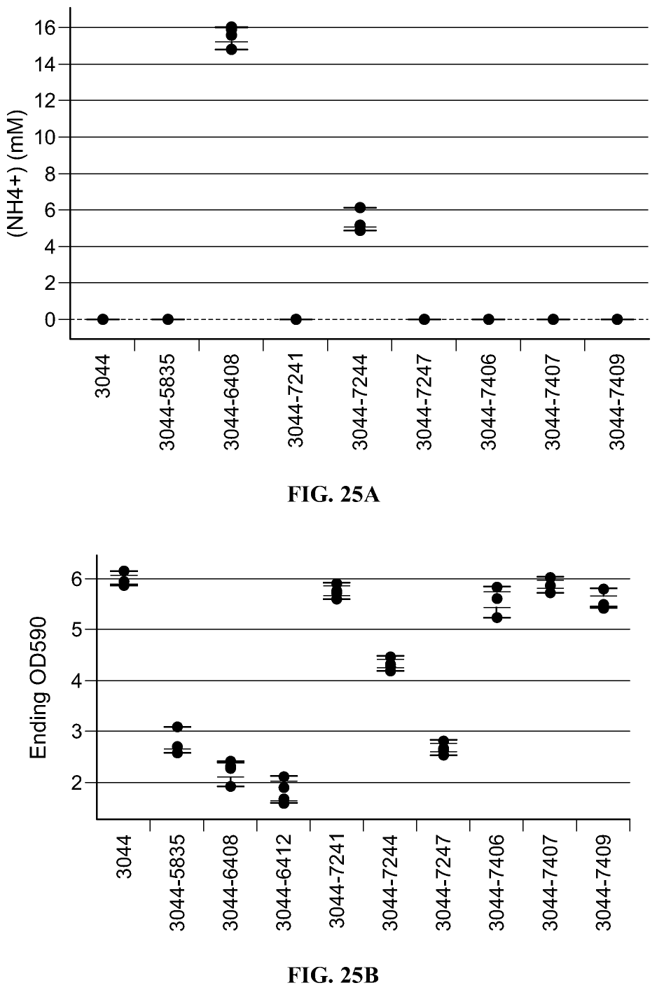

Nitrogenase activity of the various CI1666 and CI3044 strains was evaluated via acetylene reduction assay (FIGS. 22 and 24). ARA assay for the Azospirillum assay was run in semi-solid media, which allowed a gradient of oxygen levels in growth media, whereas the C13044 ARA assay had 1.5% oxygen. Strains were streaked on tryptic soy agar plates and incubated until colonies appeared. The grown colonies were scraped and resuspended in 6 ml of phosphate buffered saline. After normalizing the cell suspension to OD 0.8, 900 ul of cell suspension was inoculated on top of a 1 ml Fahreus semi-solid media. Vials of media and cell suspension were capped, injected with 700 ul of acetylene, and incubated for 3 days at 30° C. At the end of 3 days, 2 ml gas of the headspace was analyzed using GC-MS, to measure the amount of ethylene produced. The ARA data for CI1666 were consistent with the GFP expression data. The mutations in K23 and M164 residues showed nitrogenase activity, whereas the K132D mutation showed no nitrogenase activity. In the CI3044 strain, only the K21E mutation led to nitrogenase activity under rich nitrogen. This strain also showed high levels of ammonium production (FIG. 25).

In vitro assays suggested that the conserved K23 residue was important for self-repression of NifA activity under rich nitrogen conditions, as mutations to acidic residues prevented the self-repression in both Azospirillum and Paraburkholderia. In comparison, the M164 residue seemed to be important for self-repression of NifA activity under rich nitrogen conditions for the Azospirillum strain.

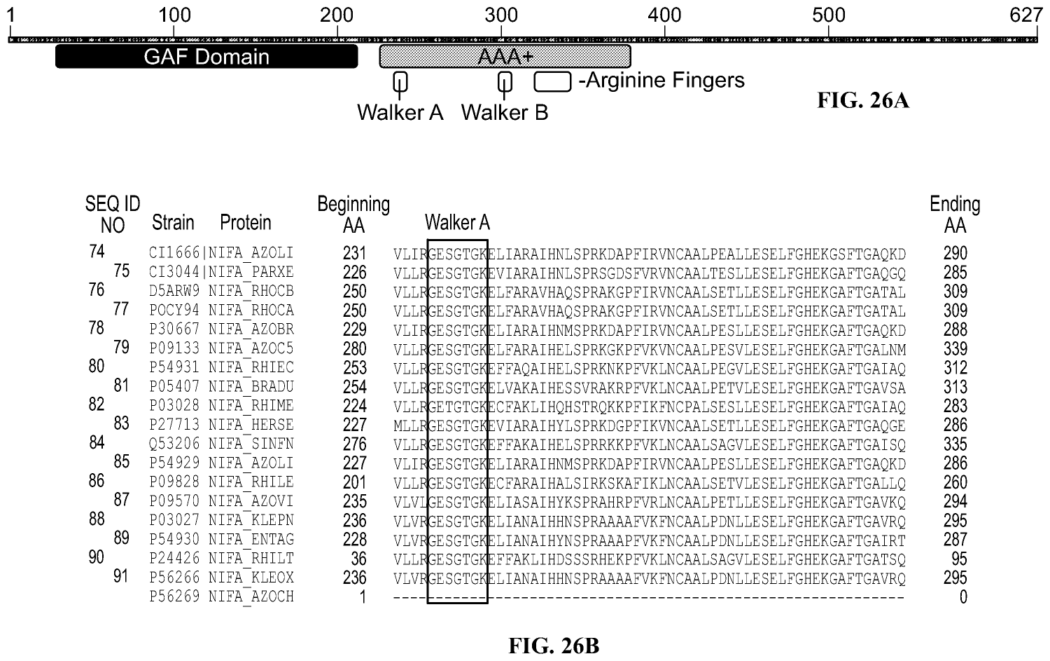

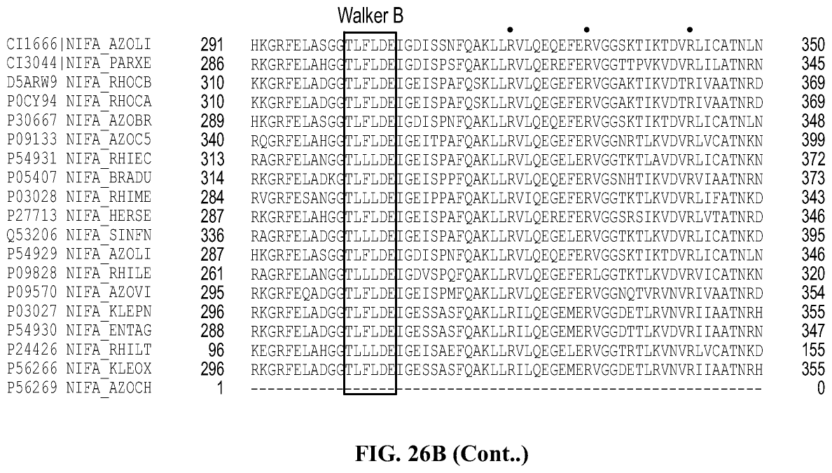

NifA belongs to the H2 insert clade of AAA+ proteins, which convert chemical energy from the hydrolysis of ATP into mechanical energy through conformational change to activate sigma-54 driven gene transcription. (See, for example Yousuf et al. (2022) Crit. Rev. Biochem. Mol. Biol, 57:2, 156-187). Domain prediction algorithms, such as ScanProsite (De Castro et al. Nucleic Acids Res. 2006; 34:W362-5) and SMART (Letunic et al. (2017) Nucleic Acids Res doi: 10.1093/nar/gkx922), mapped the GAF domain of CI1666 to residues 30-212, and mapped the central AAA+ domain to F227-L378 (FIG. 26A). The AAA⁺ domain can be divided into conserved subdomains, called Walker A, Walker B, and Arginine finger, that together bind and hydrolyze ATP. An alignment of various central AAA+ domains was completed using SEQ ID Nos: 74-91 and showed conservations of the Walker A and Walker B subdomains (FIG. 26B). The N-terminal GAF domain allows NifA to respond to small molecules (ATP and 2-oxo-glutarate) and signaling molecules (GlnB) by triggering a conformational change and regulating its own activity in organisms that lack the NifL gene (Sotomaior et al. (2012). Braz J Med Biol Res December; 45(12):1135-40). Under nitrogen limiting conditions, uridylated GlnB interacts with the GAF domain (Inaba et al. (2015). Microbiological Research, 171, 65-72). Also under nitrogen limiting conditions, uridylated GlnB prevents its own folding onto the AAA+ domain to block ATPase activity, which is mediated through the arginine fingers that bind the γ-phosphate residue of ATP (Nagy et al. (2016) J Am Chem Soc 138 (45), 15035-15045). ATP binding via arginine helps stabilize the GAFTGA motif containing loop, which engages the sigma-54 RNA polymerase to initiate gene transcription (Chen et al. (2010). Structure, 18(11), 1420-1430).

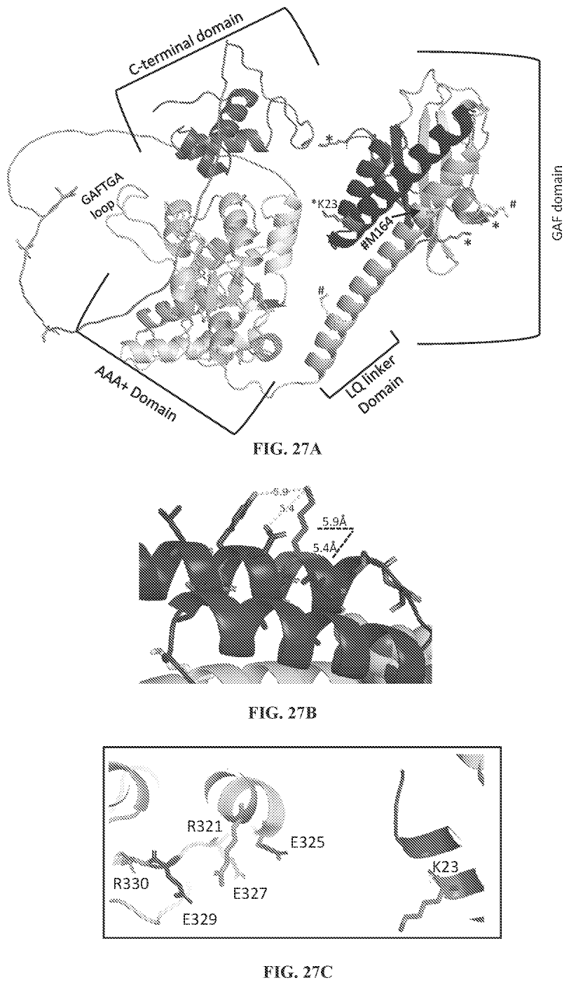

To understand how the GAF domain mutations might impact protein structure and activity, the NifA protein from CI1666 was computationally modeled using AlphaFold (Jumper et al. (2021) Nature 596, 583-589) and the identified frequently mutated lysine and methionine residues were mapped (FIG. 27A). K23, an important residue identified in the mutational analysis, was predicted to interface with the ATPase domain, whereas the other lysine and methionine residues were located on the periphery of the GAF domain. While lysines are frequently found in proteins' active sites and/or binding sites, and the positively charged amino group on the lysine side-chain is often involved in salt-bridges where it pairs with a negatively charged amino acid such as aspartate, the Alphafold predicted protein structure indicated that K23 did not form hydrogen bonds with neighboring residues (FIG. 27B). This suggested that K23 might interact with more distant residues during protein conformation changes. Furthermore, the K23 localized in the same plane as the arginine fingers and a cluster of aspartate residue (E325, E327, E329), which could form hydrogen bonding with K23 (FIG. 27C). Therefore, the predicted structure of NifA suggested a role for K23 in regulating the ATPase activity of the AAA+ domain, presumably through hydrogen bonding induced under nitrogen rich conditions.

### Example 5—Identifying Universal and Causal SNPs in the GAF Domain of NifA that Removes Self-Inhibition

The presence of methionine residues on the outward facing part of the GAF domain suggests a role for these residues in sensing oxygen levels. Work described elsewhere suggested that the sulfur in the methionine side chain could serve as an oxygen sensor (Lim et al. (2019). Neurochemical research, 44(1), 247-257). Since self-repression of NifA is regulated by nitrogen and oxygen levels, the oxygen may be sensed through one or more of the methionine residues in the GAF domain. Further studies are conducted to determine whether substituting such residues with other amino acids lacking sulfur in their side chains renders NifA more resilient to increasing oxygen levels. For example, the methionine corresponding to position 164 of SEQ ID NO: 72 or 73 in a NifA homolog is substituted to a residue lacking sulfur in the side chain. Sulfur lacking amino acid residues can include arginine, histidine, lysine, aspartic acid, glutamic acid, serine, threonine, asparagine, glutamic acid, glycine, proline, alanine, isoleucine, leucine, phenylalanine, tryptophan, tyrosine, and/or valine. In some cases, the substituted amino acid is selected from isoleucine and threonine.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An engineered microbe comprising one or more genetic modifications in a gene encoding a NifA polypeptide, wherein the engineered microbe fixes nitrogen in the presence of exogenous nitrogen and oxygen.

2. The engineered microbe of claim 1, wherein the NifA polypeptide encoded by the gene with one or more genetic modifications exhibits increased transcriptional activation of nitrogen fixation genes in the presence of nitrogen and oxygen, relative to the transcriptional activation of nitrogen fixation genes of a wild-type NifA polypeptide in the presence of nitrogen and oxygen.

3. The engineered microbe of claim 1, wherein the exogenous nitrogen comprises ammonium, nitrate, urea, or glutamine.

4. The engineered microbe of claim 3, wherein the NifA polypeptide encoded by the gene with one or more genetic modifications overcomes ammonium inhibition in the presence of nitrogen.

5. (canceled)

6. The engineered microbe of claim 4, wherein the NifA polypeptide encoded by the gene with one or more genetic modifications comprises:
a) a substitution at one or more amino acid positions corresponding to amino acids 23 or 164 of SEO ID NO: 15 or at homologous amino acid position(s) in a homolog thereof;
b) a substitution at an amino acid position corresponding to amino acid 23 of SEQ ID NO: 15, wherein the amino acid corresponding to position 23 of SEQ ID NO: 15 or at a homologous amino acid position in a homolog thereof is substituted with a non-positively charged amino acid (e.g., with the amino acid D or E);
c) a substitution at an amino acid position corresponding to amino acids 23 and 164 of SEQ ID NO: 15, wherein the amino acid corresponding to position 23 of SE ID NO: 15 is substituted with the amino acid D or E, and wherein the amino acid corresponding to position 164 of SEQ ID NO: 15 or at a homologous amino acid position in a homolog thereof is substituted with the amino acid I, L, or T;
d) a substitution at one or more amino acid positions corresponding to amino acids 7, 21, 34, 42, 93, 108, 116, 122, 159, 166, 178, 185, 186, or 196 of SEQ ID NO: 14 or at one or more homologous positions in a homolog thereof;
e) a substitution at an amino acid position corresponding to amino acid 185 of SEQ ID NO: 14 or at one or more homologous positions in a homolog thereof, wherein the amino acid corresponding to position 185 of SEQ ID NO: 14 or at a homologous amino acid position in a homolog thereof is substituted with the amino acid T;
f) a substitution at an amino acid position corresponding to amino acid 186 of SEQ ID NO: 14 or at one or more homologous positions in a homolog thereof, wherein the amino acid corresponding to position 186 of SEQ ID NO: 14 or at a homologous amino acid position in a homolog thereof is substituted with the amino acid R;
g) a substitution at an amino acid position corresponding to amino acid 196 of SEQ ID NO: 14 or at one or more homologous positions in a homolog thereof, wherein the amino acid corresponding to position 196 of SEQ ID NO: 14 or at a homologous amino acid position in a homolog thereof is substituted with the amino acid V;
h) a deletion of amino acids in the NifA polypeptide corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200; or a deletion of the GAF domain of the NifA polypeptide; or
i) an insertion of a regulatory element operably linked to the gene encoding the NifA polypeptide.

7. The engineered microbe of claim 6, wherein the microbe is:
a) a strain of Azospirillum lipoferum deposited in ATCC under Accession No. PTA-127320 or PTA-127323, or
b) a strain of Paraburkholderia xenovorans deposited in ATCC under Accession No. PTA-127325 or PTA-127319.

8.-50. (canceled)

51. The engineered microbe of claim 6, wherein the NifA polypeptide further comprises:
a) a deletion of amino acids corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200 or at homologous amino acid positions in a homolog thereof; or
b) a deletion of the GAF domain of the NifA polypeptide.

52. (canceled)

53. The engineered microbe of claim 1, wherein the microbe further comprises:
a) one or more genetic modifications in a nitrogen fixation and/or a nitrogen assimilation pathway;
b) one or more genetic modifications in a nitrogen fixation and/or the nitrogen assimilation pathway that result in altered activity of NifH, GlnK, GlnD, GlnE, or a combination thereof; or
c) one or more genetic modifications in a nitrogen fixation and/or the nitrogen assimilation pathway comprising a deleted glnK gene, a glnD gene encoding a GlnD polypeptide lacking a UTase domain, a glnE gene encoding a GlnE polypeptide lacking an AR domain, or a combination thereof.

54. (canceled)

55. (canceled)

56. The engineered microbe of 53, wherein the one or more genetic modifications comprises an insertion of a regulatory element or an insertion of a promoter.

57.-67. (canceled)

68. The engineered microbe of claim 56, wherein the regulatory element is derived from a microbe of the same species as the engineered microbe or a microbe of the same genus as the engineered microbe.

69. (canceled)

70. The engineered microbe of claim 56, wherein the regulatory element is derived from a microbe of a different species than the engineered microbe or a microbe of a different genus than the engineered microbe.

71. (canceled)

72. The engineered microbe of claim 1, wherein the engineered microbe is an engineered bacterium.

73. The engineered microbe of claim 72, wherein the engineered microbe is a Proteobacterium, or is selected from the group consisting of: Paraburkholderia spp., Azospirillum spp., and Herbaspirillum spp.

74.-76. (canceled)

77. A composition, comprising a engineered microbe comprising one or more genetic modifications in a gene encoding a NifA polypeptide, wherein the engineered microbe fixes nitrogen in the presence of exogenous nitrogen and oxygen, and a plant seed.

78. (canceled)

79. The composition of claim 77, wherein the plurality of engineered microbes are coated onto the plant seed or applied into furrows in which seeds of the plant are planted.

80. (canceled)

81. The composition of claim 77, wherein the plant seed is a non-leguminous plant seed: a cereal plant seed; or a seed of a plant selected from the group consisting of barley, canola, corn, peanut, rice, sorghum, soybean, turfgrass, and wheat.

82. (canceled)

83. (canceled)

84. A method of increasing an amount of ammonium production of a microbe, the method comprising engineering the microbe to include one or more genetic modifications in a gene encoding a NifA polypeptide, wherein the engineered microbe fixes nitrogen in the presence of a nitrogen and an oxygen.

85. The method of claim 84, wherein the NifA polypeptide encoded by the gene with one or more genetic modifications exhibits increased transcriptional activation of nitrogen fixation genes in the presence of nitrogen and oxygen relative to that of a wild-type NifA polypeptide in the presence of nitrogen and oxygen.

86-91. (canceled)

92. The method of claim 84, wherein the NifA polypeptide encoded by the gene with one or more genetic modifications comprises:
a) a substitution at one or more amino acid positions corresponding to amino acids 7, 21, 34, 42, 93, 108, 116, 122, 159, 166, 178, 185, 186, or 196 of SEQ ID NO: 14 or at one or more homologous positions in a homolog thereof; or
b) a substitution at one or more amino acid positions corresponding to amino acids 16, 23, 26, 28, 37, 65, 72, 93, 96, 123, 158, 164, 171, 183, or 209 of SEQ ID NO: 15 or at homologous amino acid position(s) in a homolog thereof.

93-114. (canceled)

115-132. (canceled)

133. The method of claim 84, wherein the NifA polypeptide further comprises:
a) a deletion of amino acids corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200 or at homologous amino acid positions in a homolog thereof; or
b) a deletion of the GAF domain of the NifA polypeptide.

134. (canceled)

135. The method of claim 84, wherein the microbe further comprises:
a) one or more genetic modifications in a nitrogen fixation and/or a nitrogen assimilation pathway;
b) one or more genetic modifications in a nitrogen fixation and/or the nitrogen assimilation pathway that result in altered activity of NifH GlnK, GlnD, GlnE, or a combination thereof;
c) one or more genetic modifications in a nitrogen fixation and/or the nitrogen assimilation pathway comprising a deleted glnK gene, a glnD gene encoding a GlnD polypeptide lacking a UTase domain, a glnE gene encoding a GlnE polypeptide lacking an AR domain, or a combination thereof;
d) one or more genetic modifications in a nitrogen fixation and/or a nitrogen assimilation pathway, wherein the one or more genetic modifications comprises an insertion of a regulatory element;
e) one or more genetic modifications comprising a deletion of amino acids in the NifA polypeptide corresponding to the following amino acids of SEQ ID NO: 14: 2-23, 2-24, 2-51, 2-75, 2-105, 2-139, 2-156, 2-167, 2-176, 2-202, 2-252, 186-196, 188-198, or 186-200; or a deletion of the GAF domain of the NifA polypeptide; or
f) one or more genetic modifications comprising an insertion of a regulatory element operably linked to the nifA gene.

136.-139. (canceled)

140. The method of claim 135, wherein the microbe further comprises one or more genetic modifications comprising an insertion of a regulatory element operably linked to the nifA gene, and wherein the regulatory element is a promoter.

141.-145. (canceled)

146. The method of claim 140, wherein the regulatory element is a constitutive promoter, an inducible promoter, or a synthetic promoter.

147.-149. (canceled)

150. The method of claim 140, wherein the regulatory element is derived from a microbe of the same species as the method or from a microbe of the same genus as the method.

151. (canceled)

152. The method of claim 140, wherein the regulatory element is derived from a microbe of a different species than the method or from a microbe of a different genus than the method.

153. (canceled)

154. The method of claim 84, wherein the microbe is an engineered bacterium.

155. The method of claim 154, wherein the engineered bacterium is a Proteobacterium or is selected from the group consisting of: Paraburkholderia spp., Azospirillum spp., and Herbaspirillum spp.

156.-165. (canceled)
